(19) United States
(12) Patent Application Publication
(10) Pub. No.: US 2010/0279892 A1
(43) Pub. Date: Nov. 4, 2010

(54) METHODS TO DETERMINE IF A SUBJECT WILL RESPOND TO A BCR-ABL INHIBITOR

(76) Inventors: Shannon K. McWeeney, Portland, OR (US); Michael W.N. Deininger, Portland, OR (US)

Correspondence Address: Klarquist Sparkman LLP, 121 SW Salmon Street, Suite 1600, Portland, OR, 97204, US

(21) Appl. No.: 12/746,475

(22) PCT Filed: Dec. 5, 2008
(86) PCT No.: PCT/US08/85724

§ 371 Date: Jun. 4, 2010

Related U.S. Application Data
(60) Provisional application No. 61/005,703, filed on Dec. 7, 2007.

(51) Int. Cl. C12Q 1/68; C40B 30/04; C40B 40/06

(52) U.S. Cl. 506/9; 435/6; 506/16

(57) ABSTRACT

Methods are provided for determining if a subject of interest will respond to treatment with BCR-ABL inhibitor, comprising. The method includes quantitating expression of a plurality of genes in CD34+ cells isolated from the subject. Expression of the plurality of genes in the subject of interest is compared to a control. Altered expression of the plurality of genes in as compared to the control indicates that the subject of interest will respond to treatment with the BCR-ABL inhibitor. Arrays are also provided.

### PRIORITY CLAIM

The application claims the benefit of U.S. Provisional Application No. 61/005,703, filed Dec. 7, 2007, which is incorporated by reference herein in its entirety.

### STATEMENT OF GOVERNMENT SUPPORT

This invention was made with United States Government support under grant HL082978-01, awarded by the National Institutes of Health. The Government has certain rights in the invention.

### FIELD

This relates to the field of cancer, specifically to methods for determining if a subject with chronic myelogenous leukemia is amenable to treatment with a BCR-ABL inhibitor, as well as arrays that can be used for such methods.

### BACKGROUND

Chronic myeloid leukemia (CML) is caused by BCR-ABL, a constitutively active tyrosine kinase that results from a (9;22) translocation. This translocation is cytogenetically visible as the Philadelphia chromosome (Ph) (Deininger et al., Blood 2000; 96:3343-3356). Most patients are diagnosed in the chronic phase, which is characterized by expansion of myeloid cells. If left untreated the disease progresses to accelerated phase or blast crisis, an acute leukemia with a poor prognosis. Imatinib, a small molecule inhibitor of the ABL kinase has revolutionized CML therapy (Deininger et al., Blood 2005; 105:2640-2653). A recent update of a study of newly diagnosed patients with CML in chronic phase treated with imatinib as initial therapy, showed an 87% cumulative rate of complete cytogenetic response (complete cytogenetic response (CCyR), 0% Ph+metaphases) and a projected overall survival of 89% with 60 months of follow-up (Druker et al., N. Engl. J. Med. 2006; 355:2408-2417). Despite these impressive results, major challenges remain

For example, approximately 16% of patients lost their response, including 7% who progressed to accelerated phase or blast crisis. In addition, approximately 14% of patients exhibited primary cytogenetic resistance, wherein they failed to attain a major cytogenetic response (<35% Ph+ metaphases) at 12 months. These patients had a 19% risk of progression to accelerated phase or blast crisis at 5 years, compared to only 3% of patients who were in complete cytogenetic response after 12 months of therapy (Druker et al., N. Engl. J. Med. 2006; 355:2408-2417). The administration of a BCR-ABL inhibitor in these subjects delays the administration of an alternative, more-effective individualized therapy, incurs expenses for an ineffective therapeutic protocol, and can result in the subject having a blast crisis. Thus, need remains to be able to identify patients with primary cytogenetic resistance, and to be able to identify those subjects in which the BCR-ABL inhibitor becomes ineffective.

### SUMMARY

Methods are provided for determining if a subject of interest will respond to treatment with BCR-ABL inhibitor, such as imatinib. The methods include quantitating expression of a plurality of genes listed in Table 2 in CD34+ cells isolated from the subject. Expression of the plurality of genes in the subject of interest is compared to a control. Altered expression of the plurality of genes in as compared to the control indicates that the subject of interest will respond to treatment with the BCR-ABL inhibitor. The methods can be used to identify subjects with primary cytogenetic resistance. The methods can also be used to identify those subjects with CML wherein a BCR-ABL inhibitor becomes ineffective.

In some examples, the methods include detecting expression of chemotherapy sensitivity-related molecules at either the nucleic acid level or protein level. In another example, the methods include determining whether a gene expression profile from the subject indicates that the subject with achieve a cytogenetic response to a BCR-ABL inhibitor by using an array of molecules. In one example, the array includes oligonucleotides complementary to all genes listed in Table 2.

Also disclosed are kits, including arrays, for predicting response of a subject with CML to a BCR-ABL inhibitor. For example, an array can include one or more of the genes listed in Table 2. Arrays can include other molecules, such as positive and negative controls.

The foregoing and other features and advantages will become more apparent from the following detailed description of several embodiments, which proceeds with reference to the accompanying Figures.

### BRIEF DESCRIPTION OF THE FIGURES

FIG. 1 is a plot of an unsupervised cluster analysis that was performed on the training set (N=36). Patients who subsequently achieved CCyR partially separated from patients with >65% Ph-positive metaphases after 12 months of imatinib therapy.

FIG. 2 is a plot of an unsupervised cluster analysis of the validation set (N=23), using the minimal list of 75 probe sets (68 genes) derived from the training set. Non-responders and responders are separated.

FIG. 3 is a plot of the results of a Metacore Database® analysis of the protein-protein interactions among the members of the classifier and identified a highly significant interaction subnetwork (p<4.85-36), which included two ANGPT1 signaling related pathways (both part of Metacore® Curated Map 532). The key classifier node that linked both of these pathways was ANGPT1. Circles indicate genes up-regulated in non-responders.

FIGS. 4A and B are bar graphs of the results of meta-analysis to assess overlap between the 885 probe sets differentially expressed between responders and non-responders in the training set, and two previously published data sets. The histograms represent the results of 10,000 simulations to determine the probability of seeing a concordance equal to or greater than what we observed (FIG. 4A) Comparison with a gene profile of blastic vs. chronic phase reported by Zheng et al. (FIG. 4B) Comparison with a gene profile of patients with short vs. long duration of chronic phase on treatment with non-imatinib therapy reported by Yong et al.

FIG. 5A-C are dot plots of an exemplary sorting strategy to select CD34+ cells from frozen mononuclear cells (MNC). Viable cells were initially enriched by removal of dead cells by immunomagnetic beads and columns, followed by staining for propidium iodide, CD34 and CD45. (FIG. 5A) Forward scatter (FSC-A) vs. side scatter (SSC-A) plot showing viable cells (P1 gate) and ungated debris and non-viable cells. (FIG. 5B) The sorting gate for CD34+/dim CD45+ cells (P4) includes approximately 1% of total viable cells. (FIG. 5C) Reanalysis after cell sorting shows an enriched CD34+/dim CD45+ cell population comprising approximately 91% of sorted cells.

FIG. 6 is a plot of the classifier derived from the training set applied to an independent cohort of 23 newly diagnosed patients (validation set). The frame indicates the 6 patients who did not achieve a major cytogenetic response within 12 months of imatinib therapy.

FIG. 7 is a representation of the clustering of transcripts based on shared transcription factor (TF) binding sites in the 2 kb upstream region for transcripts in the classifier.

FIG. 8 is a set of histograms and bar graphs showing mononuclear cells from a patient with primary cytogenetic resistance that were incubated with 5 μM or 50 nm dasatinib, respectively. Total phosphotyrosine and phosphor-CrkL were measure by FACS. The data suggest the cells are independent of BCR-ABL.

FIG. 9A-C is a set of graphs dot plots and digital images of Western blots showing viable cells and phosphotyrosine content following treatment with a BCR-ABL inhibitor. (FIG. 9A) Lin−/CD34+/CD38 and Lin−/CD34+/CD38− cells from a newly diagnosed patient with CML and a normal and a normal control were grown in serum free media and physiological concentrations of cytokines in the presence of 5 microM imatinib and the total number of viable cells measured over time. (FIG. 9B) After 2 hours, immunoblot analysis of cellular extracts for Crk1 phosphorylation was preformed. (FIG. 9C) Aliquots from the same cultures were analyzed by FACS analysis for total cellular phosphotyrosine content. Results were identical after 96 hours of culture.

FIG. 10A-C is a set of bar graphs showing the effect of fibronectin and intergrin on 34+ cells from newly diagnosed CML patients that were cultured for 96 hours in the presence or absence of fibronectin Beta 1-integrin activating or blocking antibodies and imatinib (5 micoM) added at the initiation of culture. (FIG. 10A) Adhesion under the carious conditions. (FIG. 10B) Fold expansion of viable cells. (FIG. 10C) Recovery of CFU-GM.

FIG. 11 is a set of bar graphs showing the effect of a stromal cell layer on CD34+ cells from a newly diagnosed patient that were culture for 96 hours in the presence or absence of a stromal cell layer and the presence or 50 nM dasatinib. After the culture cells were plated in semisolid media and CFU-GM counted after 2 weeks.

FIGS. 12A and B is a set of bar graphs showing cytokine secretion. (FIG. 12A) Mononuclear cells from 3 patients with chronic phase CML were cultured in 2 microM imatinib, 50 nM dasatinib or 1 microM SGX70393 in the presence of IL-3, SCF, GM-CSF and IL-6. (FIG. 12B) In a separate set of experiments, mononuclear cells from CML patients were grown in the presence and absence of 2 microM imatinib and 1 microM SGX790393 in the presence of IL-3, SCF and GM-CSF (all cytokines) or with one cytokine omitted from the culture as indicated.

FIG. 13A-C is a set of graphs and digital images of Western blots showing the effect of the inhibition of KIT. (FIG. 13A) Lineage-depleted cell from a newly diagnosed CML patient were grown in serum-free media and low cytokine concentrations, with inhibitors added as indicated. Concentrations were 2 microM imatinib, 50 nM dasatinib, 1 microM SGX70393 and 1 micro SU5416. (FIG. 13B) More cells expressing BCR-ABL and stimulated with SCF were treated with inhibitors and subjected to immunoblot analysis using phosphor-specific antibodies to ABL and KIT. (FIG. 13C) Cells from a newly diagnosed CML patient were sorted by FACS and treated or not with 2 microM imatinib or 1 microM SGX709393. Total cellular phosphotyrosine was measured by FACS in untreated cells, treated cells and after 3 washes in PBS.

FIG. 14 is a schematic of potential mechanisms underlying disease persistence in CML.

FIG. 15A-DD is referred to in the text as Table 6.

### DETAILED DESCRIPTION

### I. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a nucleic acid molecule” includes single or plural nucleic acid molecules and is considered equivalent to the phrase “comprising at least one nucleic acid molecule.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

Although methods and materials similar or equivalent to those described herein can be used in the practice or testing of the present disclosure, suitable methods and materials are described below. The materials, methods, and examples are illustrative only and not intended to be limiting.

To facilitate review of the various embodiments of this disclosure, the following explanations of specific terms are provided:

Accuracy: The degree of closeness of a measured, calculated, or predicted outcome to its actual outcome, for example in a prediction of whether or not someone diagnosed with CML will respond to a BCR-ABL inhibitor.

Administration: To provide or give a subject an agent, such as a BCR-ABL inhibitor, by any effective route. Exemplary routes of administration include, but are not limited to, oral, injection (such as subcutaneous, intramuscular, intradermal, intraperitoneal, and intravenous), sublingual, rectal, transdermal, intranasal, vaginal and inhalation routes.

Amplifying a nucleic acid molecule: To increase the number of copies of a nucleic acid molecule, such as a gene or fragment of a gene, such as the genes listed in Table 2. The resulting products are called amplification products.

An example of in vitro amplification is the polymerase chain reaction (PCR), in which a biological sample obtained from a subject (such as a sample containing tumor cells or CD34+ cells) is contacted with a pair of oligonucleotide primers, under conditions that allow for hybridization of the primers to a nucleic acid molecule in the sample. The primers are extended under suitable conditions, dissociated from the template, and then re-annealed, extended, and dissociated to amplify the number of copies of the nucleic acid molecule. Other examples of in vitro amplification techniques include quantitative real-time RT-PCR, strand displacement amplification (see U.S. Pat. No. 5,744,311); transcription-free isothermal amplification (see U.S. Pat. No. 6,033,881); repair chain reaction amplification (see WO 90/01069); ligase chain reaction amplification (see EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134).

Animal: A living multicellular vertebrate organism, a category that includes, for example, mammals and birds. A “mammal” includes both human and non-human mammals. “Subject” includes both human and animal subjects.

Antibody: A polypeptide ligand comprising at least a light chain or heavy chain immunoglobulin variable region which specifically recognizes and binds an epitope of an antigen, such as any of the proteins encoded by the genes listed in Table 2 or a fragment thereof. Antibodies are composed of a heavy and a light chain, each of which has a variable region, termed the variable heavy (VH) region and the variable light (VL) region. Together, the VH region and the VL region are responsible for binding the antigen recognized by the antibody. This includes intact immunoglobulins and the variants and portions of them well known in the art, such as Fab' fragments, F(ab)'2 fragments, single chain Fv proteins (“scFv”), and disulfide stabilized Fv proteins (“dsFv”). The term also includes recombinant forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, Immunology, 3rd Ed., W.H. Freeman & Co., New York, 1997.

Array: An arrangement of molecules, such as biological macromolecules (such as peptides or nucleic acid molecules) or biological samples (such as tissue sections), in addressable locations on or in a substrate. A “microarray” is an array that is miniaturized so as to require or be aided by microscopic examination for evaluation or analysis. Arrays are sometimes called DNA chips or biochips.

The array of molecules (“features”) makes it possible to carry out a very large number of analyses on a sample at one time. In certain example arrays, one or more molecules (such as an oligonucleotide probe) will occur on the array a plurality of times (such as twice), for instance to provide internal controls. The number of addressable locations on the array can vary, for example from at least one, to at least 6, to at least 10, at least 20, at least 30, at least 50, at least 75, at least 100, at least 150, at least 200, at least 300, at least 500, least 550, at least 600, at least 800, at least 1000, at least 10,000, or more. In particular examples, an array includes nucleic acid molecules, such as oligonucleotide sequences that are at least 15 nucleotides in length, such as about 15-40 nucleotides in length. In particular examples, an array includes oligonucleotide probes or primers which can be used to detect genes associated with prediction of CCyR, such as at least one of those listed in Table 2, such as at least 6, at least 10, at least 20, at least 30, at least 50, or at least 60, of the sequences of the genes listed in Table 2. In an example, the array is a commercially available such as a U133 Plus 2.0 oligonucleotide array from AFFYMETRIX® (AFFYMETRIX®, Santa Clara, Calif.).

Within an array, each arrayed sample is addressable, in that its location can be reliably and consistently determined within at least two dimensions of the array. The feature application location on an array can assume different shapes. For example, the array can be regular (such as arranged in uniform rows and columns) or irregular. Thus, in ordered arrays the location of each sample is assigned to the sample at the time when it is applied to the array, and a key may be provided in order to correlate each location with the appropriate target or feature position. Often, ordered arrays are arranged in a symmetrical grid pattern, but samples could be arranged in other patterns (such as in radially distributed lines, spiral lines, or ordered clusters). Addressable arrays usually are computer readable, in that a computer can be programmed to correlate a particular address on the array with information about the sample at that position (such as hybridization or binding data, including for instance signal intensity). In some examples of computer readable formats, the individual features in the array are arranged regularly, for instance in a Cartesian grid pattern, which can be correlated to address information by a computer.

Protein-based arrays include probe molecules that are or include proteins, or where the target molecules are or include proteins, and arrays including nucleic acids to which proteins are bound, or vice versa. In some examples, an array contains antibodies to proteins associated with prediction of CCyR, such as any combination of those listed in Table 2, such as at least 1, at least 6, at least 10, at least 20, at least 30, at least 50, or at least 60, of the proteins encoded by the genes listed Table 2.

Bcr-Abl: A fusion gene that is the result of a reciprocal translocation between chromosomes 9 and 22 [t(9;22)], cytogenetically evident as the Philadelphia chromosome (Ph), and encoding a constitutively active tyrosine kinase. The Bcr-Abl gene is derived from relocation of the portion of c-ABL gene from chromosome 9 to the portion of BCR gene locus on chromosome 22. Bcr-Abl hybrid genes produce p230, p210, and p185 fusion proteins (where p refers to the approximate molecular weight in kilodaltons, with the size depending on the breakpoint in BCR locus). Bcr-Abl is an oncogene that is responsible for the transformation of hematopoietic stem cells and the symptoms of chronic myeloid leukemia (CML) and Philadelphia (Ph+) acute lymphoblastic leukemia (ALL), and includes any Bcr-Abl gene, cDNA, RNA, or protein from any organism, such as a mammal. Bcr-Abl nucleic acid and protein sequences are known in the art.

Bcr-Abl inhibitor or Abl kinase inhibitor: An agent that can significantly reduce the biological activity of Bcr-Abl and/or Abl kinase alone or in the presence of another molecule, such as a reduction of Bcr-Abl and/or Abl kinase activity at least 20%, at least 80%, or at least 99%. Examples of such inhibitors include imatinib, AMN107 (nilotinib), dasatinib, NS-187, ON012380, Bosutinib (SKI-606), INNO-406 (NS-187), MK-0457 (VX-680), SGX70393 and BMS-354825.

Binding or stable binding: An association between two substances or molecules, such as the hybridization of one nucleic acid molecule to another (or itself), the association of an antibody with a peptide, or the association of a protein with another protein or nucleic acid molecule. An oligonucleotide molecule binds or stably binds to a target nucleic acid molecule if a sufficient amount of the oligonucleotide molecule forms base pairs or is hybridized to its target nucleic acid molecule, to permit detection of that binding. For example a probe or primer specific for a nucleic acid molecule of interest can stably bind to the nucleic acid molecule encoding the protein of interest.

Binding can be detected by any procedure known to one skilled in the art, such as by physical or functional properties of the target:oligonucleotide complex. For example, binding can be detected functionally by determining whether binding has an observable effect upon a biosynthetic process such as expression of a gene, DNA replication, transcription, translation, and the like.

Physical methods of detecting the binding of complementary strands of nucleic acid molecules, include but are not limited to, such methods as DNase I or chemical footprinting, gel shift and affinity cleavage assays, Northern blotting, dot blotting and light absorption detection procedures. For example, one method involves observing a change in light absorption of a solution containing an oligonucleotide (or an analog) and a target nucleic acid at 220 to 300 nm as the temperature is slowly increased. If the oligonucleotide or analog has bound to its target, there is a sudden increase in absorption at a characteristic temperature as the oligonucleotide (or analog) and target disassociate from each other, or melt. In another example, the method involves detecting a signal, such as a detectable label, present on one or both nucleic acid molecules (or antibody or protein as appropriate).

The binding between an oligomer and its target nucleic acid is frequently characterized by the temperature (Tₘ) at which 50% of the oligomer is melted from its target. A higher (Tₘ) means a stronger or more stable complex relative to a complex with a lower (Tₘ).

Cancer: Malignant neoplasm that has undergone characteristic anaplasia with loss of differentiation, increase rate of growth, invasion of surrounding tissue, and is capable of metastasis. In cancer treatment, “chemotherapy” or “administration of an anti-cancer agent” refers to the administration of one or a combination of compounds or physical processes (such as irradiation) to kill or slow the reproduction of rapidly multiplying cells. Anti-neoplastic chemotherapeutic agents include those known by those skilled in the art, including, but not limited to: 5-fluorouracil (5-FU), azathioprine, cyclophosphamide, antimetabolites (such as Fludarabine), antineoplastics (such as Etoposide, Doxorubicin, methotrexate, and Vincristine), carboplatin, cis-platinum and the taxanes, such as taxol. BCR-ABL inhibitors are chemotherapeutic agents. One of skill in the art can readily identify a chemotherapeutic agent of use (see for example, Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed., 2000 Churchill Livingstone, Inc; Baltzer and Berkery. (eds): Oncology Pocket Guide to Chemotherapy, 2nd ed. St. Louis, Mosby-Year Book, 1995; Fischer Knobf, and Durivage (eds): The Cancer Chemotherapy Handbook, 4th ed. St. Louis, Mosby-Year Book, 1993). “Chemotherapy-resistant disease” is a cancer that is not significantly responsive to administration of one or more chemotherapeutic agents, such as a BCR-ABL inhibitor. A “non-cancerous tissue” is a tissue (or cells) from the same organ wherein the malignant neoplasm formed, but does not have the characteristic pathology of the neoplasm. Generally, noncancerous tissues (or cells) appear histologically normal. A “normal tissue” is tissue from an organ, wherein the organ is not affected by cancer or another disease or disorder of that organ. A “cancer-free” subject has not been diagnosed with a cancer of that organ and does not have detectable cancer.

CD34: A cell surface glycoprotein known as “cluster differentiation 34.” Hematopoietic stem cells express CD34. An exemplary nucleic and amino acid sequence of CD34 is GENBANK® Accession NO. NM_{—}001025109, as available Dec. 3, 2007, incorporated herein by reference in its entirety.

cDNA (complementary DNA): A piece of DNA lacking internal, non-coding segments (introns) and regulatory sequences which determine transcription. cDNA can be synthesized by reverse transcription from messenger RNA extracted from cells.

Chronic myelogenous leukemia (CML): A form of leukemia characterized by the increased and unregulated growth of predominantly myeloid cells in the bone marrow and the accumulation of these cells in the blood. CML is a clonal bone marrow stem cell disorder in which proliferation of mature granulocytes (neutrophils, eosinophils, and basophils) and their precursors is the main finding. It is a type of myeloproliferative disease associated with a characteristic chromosomal translocation called the Philadelphia chromosome. CML is caused by BCR-ABL.

CML is often divided into three phases based on clinical characteristics and laboratory findings. In the absence of intervention, CML typically begins in the chronic phase, and over the course of several years progresses to an accelerated phase and ultimately to a blast crisis. Blast crisis is the terminal phase of CML and clinically behaves like an acute leukemia. Progression from chronic phase through acceleration and blast crisis is characterized by the acquisition of new chromosomal abnormalities in addition to the Philadelphia chromosome.

Complementarity and percentage complementarity: Molecules with complementary nucleic acids form a stable duplex or triplex when the strands bind, (hybridize), to each other by forming Watson-Crick, Hoogsteen or reverse Hoogsteen base pairs. Stable binding occurs when an oligonucleotide molecule remains detectably bound to a target nucleic acid sequence under the required conditions.

Complementarity is the degree to which bases in one nucleic acid strand base pair with the bases in a second nucleic acid strand. Complementarity is conveniently described by percentage, that is, the proportion of nucleotides that form base pairs between two strands or within a specific region or domain of two strands. For example, if 10 nucleotides of a 15-nucleotide oligonucleotide form base pairs with a targeted region of a DNA molecule, that oligonucleotide is said to have 66.67% complementarity to the region of DNA targeted.

In the present disclosure, “sufficient complementarity” means that a sufficient number of base pairs exist between an oligonucleotide molecule and a target nucleic acid sequence (such as a genes associated with prediction of CCyR, for example any nucleic acid encoding a gene listed in Table 2) to achieve detectable binding. When expressed or measured by percentage of base pairs formed, the percentage complementarity that fulfills this goal can range from as little as about 50% complementarity to full (100%) complementary. In general, sufficient complementarity is at least about 50%, for example at least about 75% complementarity, at least about 90% complementarity, at least about 95% complementarity, at least about 98% complementarity, or even at least about 100% complementarity.

A thorough treatment of the qualitative and quantitative considerations involved in establishing binding conditions that allow one skilled in the art to design appropriate oligonucleotides for use under the desired conditions is provided by Beltz et al. Methods Enzymol. 100:266-285, 1983, and by Sambrook et al. (ed.), Molecular Cloning: A Laboratory Manual, 2nd ed., vol. 1-3, Cold Spring Harbor Laboratory Press, Cold Spring Harbor, N.Y., 1989.

Contacting: Placement in direct physical association, including both a solid and liquid form. Contacting can occur in vitro with isolated cells or tissue or in vivo by administering to a subject.

Control: A reference standard. A control can be a standard value or the amount of a substance, such as a specific protein or mRNA in a control, such as the amount expressed in CD34+ cells in a subject with CML that responds to a BCR-ABL inhibitor, such as a subject who is in complete cytogenetic remission (complete cytogenetic response CCyR), or in a subject who does not have a leukemia, such as CML. A difference between a test sample and a control can be an increase or conversely a decrease. The difference can be a qualitative difference or a quantitative difference, for example a statistically significant difference. In some examples, a difference is a decrease, relative to a control, of at least about 10%, such as at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater then 500%.

Cytogenetics. An evaluation of the genetic material of subject with or believed to have cancer, such as CML. Two types of cytogenetics, “conventional” and FISH, are used to diagnose and follow the course of CML. Conventional cytogenetics is a microscopic exam of about marrow cells in a phase of cell division when chromosomes can be clearly seen and differentiated to determine if the Ph chromosome is present. In some example at least about 10 cells, such as at least 20, at least 30, at least 40, at least 50, or more cells are examined for the presence of the Ph chromosome. Methods of cytogenetic testing are well known in the art.

Cytogenetic response (CyR). A response to treatment of CML that occurs in the marrow, rather than just in the blood. There are 3 levels of cytogenetic response: 1) just plain cytogenetic response (CyR); 2) Major cytogenetic response (MCyR); and complete cytogenetic response (CCyR). If the number of Ph+chromosomes decreases at all during treatment, a cytogenetic response (CyR) is achieved; if the Ph+percentage drops to 35 percent or less, it is considered a major cytogenetic response (MCyR); 0% Ph+ is a complete cytogenetic response (CCyR). A “Complete cytogenetic response” (CCyR) it is the complete absence of leukemic (Ph+) cells in the bone marrow of CML patients by either conventional or Fluorescence in situ hybridization (FISH) cytogenetic testing.

DNA (deoxyribonucleic acid): A long chain polymer which includes the genetic material of most living organisms (some viruses have genes including ribonucleic acid, RNA). The repeating units in DNA polymers are four different nucleotides, each of which includes one of the four bases, adenine, guanine, cytosine and thymine bound to a deoxyribose sugar to which a phosphate group is attached. Triplets of nucleotides, referred to as codons, in DNA molecules code for amino acid in a polypeptide. The term codon is also used for the corresponding (and complementary) sequences of three nucleotides in the mRNA into which the DNA sequence is transcribed.

Determining expression of a gene product: Detection of a level of expression in either a qualitative or quantitative manner, for example by detecting nucleic acid or protein by routine methods known in the art. Non-limiting examples of methods for the detection of proteins and nucleic acids are given below in Section A.

Diagnosis: The process of identifying a disease by its signs, symptoms and results of various tests. The conclusion reached through that process is also called “a diagnosis.” Forms of testing commonly performed include blood tests, medical imaging, urinalysis, and biopsy. In some examples, a subject is diagnosed with CML.

Differential expression or altered expression: A difference, such as an increase or decrease, in the amount of messenger RNA, the conversion of mRNA to a protein, or both. In some examples, the difference is relative to a control or reference value, such as an amount of gene expression in tissue not affected by a disease, such as from CD34+ cells isolated from a different subject who does not have CML, or CD34+ cells from a subject with CML who is in CCyR. Detecting differential expression can include measuring a change in gene or protein expression, such as a change in expression of one or more genes or proteins. See also, “downregluated” and “upregulated,” below.

Downregulated or inactivation: When used in reference to the expression of a nucleic acid molecule, such as a gene, refers to any process which results in a decrease in production of a gene product. A gene product can be RNA (such as mRNA, rRNA, tRNA, and structural RNA) or protein. Therefore, gene downregulation or deactivation includes processes that decrease transcription of a gene or translation of mRNA. Examples of processes that decrease transcription include those that facilitate degradation of a transcription initiation complex, those that decrease transcription initiation rate, those that decrease transcription elongation rate, those that decrease processivity of transcription and those that increase transcriptional repression. Gene downregulation can include reduction of expression above an existing level. Examples of processes that decrease translation include those that decrease translational initiation, those that decrease translational elongation and those that decrease mRNA stability.

Gene downregulation includes any detectable decrease in the production of a gene product. In certain examples, production of a gene product decreases by at least 2-fold, for example at least 3-fold or at least 4-fold, as compared to a control (such an amount of gene expression in a normal cell or cell from a subject in CCyR). In several examples, a control is a relative amount of gene expression or protein expression in one or more subjects who do not have CML, or in a subject with CML who responds to treatment with a BCR-ABL inhibitor, such as a subject in CCyR.

Expression: The process by which the coded information of a gene is converted into an operational, non-operational, or structural part of a cell, such as the synthesis of a protein. Gene expression can be influenced by external signals. For instance, exposure of a cell to a hormone may stimulate expression of a hormone-induced gene. Different types of cells can respond differently to an identical signal. Expression of a gene also can be regulated anywhere in the pathway from DNA to RNA to protein. Regulation can include controls on transcription, translation, RNA transport and processing, degradation of intermediary molecules such as mRNA, or through activation, inactivation, compartmentalization or degradation of specific protein molecules after they are produced.

The expression of a nucleic acid molecule can be altered relative to a normal (wild type) nucleic acid molecule, or the level of the nucleic acid in a subject responding to a treatment. Alterations in gene expression, such as differential expression, includes but is not limited to: (1) over-expression; (2) under-expression; or (3) suppression of expression. Alternations in the expression of a nucleic acid molecule can be associated with, and in fact cause, a change in expression of the corresponding protein.

Protein expression can also be altered in some manner to be different from the expression of the protein in a normal situation, such as expression in a subject who responds to a BCR-ABL inhibitor, such as a subject in CCyR. This includes but is not necessarily limited to: (1) a mutation in the protein such that one or more of the amino acid residues is different; (2) a short deletion or addition of one or a few (such as no more than 10-20) amino acid residues to the sequence of the protein; (3) a longer deletion or addition of amino acid residues (such as at least 20 residues), such that an entire protein domain or sub-domain is removed or added; (4) expression of an increased amount of the protein compared to a control or standard amount; (5) expression of a decreased amount of the protein compared to a control or standard amount; (6) alteration of the subcellular localization or targeting of the protein; (7) alteration of the temporally regulated expression of the protein (such that the protein is expressed when it normally would not be, or alternatively is not expressed when it normally would be); (8) alteration in stability of a protein through increased longevity in the time that the protein remains localized in a cell; and (9) alteration of the localized (such as organ or tissue specific or subcellular localization) expression of the protein (such that the protein is not expressed where it would normally be expressed or is expressed where it normally would not be expressed), each compared to a control or standard. Controls or standards for comparison to a sample, for the determination of differential expression, include samples believed to be normal (in that they are not altered for the desired characteristic, for example a sample from a subject with CML who is in CCyR, or a subject without CML) as well as laboratory values, even though possibly arbitrarily set. Laboratory standards and values may be set based on a known or determined population value and can be supplied in the format of a graph or table that permits comparison of measured, experimentally determined values.

Fluorescence in situ hybridization (FISH). A cytogenetics technique that uses a fluorescent-labeled DNA probe to determine the presence or absence of a particular segment of DNA, for example the BCR-ABL gene in CML. It combines the ability to identify a specific gene or gene region (molecular) with direct visualization of the cells and/or chromosomes under the microscope (cytogenetics). In the FISH test, typically at least about 10 cells, such at least about 20, at least about 30, at least about 40, at least about 50, at least about 60, at least about 70, at least about 80, at least about 100, at least about 120, at least about 140, at least about 160, at least about 180, at least about 200, cells, such as white blood cells and/or bone marrow cells are examined. Methods of FISH detection are well known in the art.

Gene expression profile (or fingerprint): Differential or altered gene expression can be detected by changes in the detectable amount of gene expression (such as cDNA or mRNA) or by changes in the detectable amount of proteins expressed by those genes. A distinct or identifiable pattern of gene expression, for instance a pattern of high and low expression of a defined set of genes or gene-indicative nucleic acids such as ESTs; in some examples, as few as one or two genes provides a profile, but more genes can be used in a profile, for example at least 5, at least 10, at least 15, at least 20, at least 30, at least 40, at least 50, or at least 60, such as all of the genes listed in Table 2. A gene expression profile (also referred to as a fingerprint) can be linked to a tissue or cell type (such as CD34+ cells) or to other distinct or identifiable condition that influences gene expression in a predictable way. Gene expression profiles can include relative as well as absolute expression levels of specific genes, and can be viewed in the context of a test sample compared to a baseline or control sample profile (such as a sample from a subject who does not have CML, or a subject with CML that responds to an inhibitor of BCR-ABL). In one example, a gene expression profile in a subject is read on an array (such as a nucleic acid or protein array). In some examples a gene expression profile can be used to predict CCyR in a subject with CML in response to a BCR-ABL inhibitor.

Hybridization: To form base pairs between complementary regions of two strands of DNA, RNA, or between DNA and RNA, thereby forming a duplex molecule. Hybridization conditions resulting in particular degrees of stringency will vary depending upon the nature of the hybridization method and the composition and length of the hybridizing nucleic acid sequences. Generally, the temperature of hybridization and the ionic strength (such as the Na+ concentration) of the hybridization buffer will determine the stringency of hybridization. Calculations regarding hybridization conditions for attaining particular degrees of stringency are discussed in Sambrook et al., (1989) Molecular Cloning, second edition, Cold Spring Harbor Laboratory, Plainview, N.Y. (chapters 9 and 11).

In particular examples, probes or primers can hybridize to one or more molecules (such as mRNA or cDNA molecules), for example under very high or high stringency conditions.

The following is an exemplary set of hybridization conditions and is not limiting:

Very High Stringency (detects sequences
that share at least 90% identity)

| | |
|---|---|
| Hybridization: | 5x SSC at 65° C. for 16 hours |
| Wash twice: | 2x SSC at room temperature (RT) for 15 minutes |
| | each |
| Wash twice: | 0.5x SSC at 65° C. for 20 minutes each |

High Stringency (detects sequences that share at least 80% identity)

| | |
|---|---|
| Hybridization: | 5x-6x SSC at 65° C.-70° C. for 16-20 hours |
| Wash twice: | 2x SSC at RT for 5-20 minutes each |
| Wash twice: | 1x SSC at 55° C.-70° C. for 30 minutes each |

Low Stringency (detects sequences that share at least 50% identity)

| | |
|---|---|
| Hybridization: | 6x SSC at RT to 55° C. for 16-20 hours |
| Wash at least twice: | 2x-3x SSC at RT to 55° C. for 20-30 minutes each. |

Inhibiting or treating a disease: Inhibiting the full development of a disease or condition, for example, in a subject who is at risk for a disease such cancer, such as chronic myelogenous leukemia (CML). “Treatment” refers to a therapeutic intervention that ameliorates a sign or symptom of a disease or pathological condition after it has begun to develop. For example, a treatment can induce CCyR (0% Philadelphia (Ph+) metaphases) or a major cytogenetic response (<35% Philadelphia (Ph+) metaphases). The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of metastases, an improvement in the overall health or well-being of the subject, or by other clinical or physiological parameters associated with a particular disease. A “prophylactic” treatment is a treatment administered to a subject who does not exhibit signs of a disease or exhibits only early signs for the purpose of decreasing the risk of developing pathology.

Isolated: An “isolated” biological component (such as a nucleic acid molecule, protein, or cell) has been substantially separated or purified away from other biological components in the cell of the organism, or the organism itself, in which the component naturally occurs, such as other chromosomal and extra-chromosomal DNA and RNA, proteins and cells. Nucleic acid molecules and proteins that have been “isolated” include molecules (such as DNA or RNA) and proteins purified by standard purification methods. The term also embraces nucleic acid molecules and proteins prepared by recombinant expression in a host cell as well as chemically synthesized nucleic acid molecules and proteins. For example, an isolated cell, such as a cancer cell or a CD34+ cell, is one that is substantially separated from other types of cells.

Label: An agent capable of detection, for example by ELISA, spectrophotometry, flow cytometry, or microscopy. For example, a label can be attached to a nucleic acid molecule or protein, thereby permitting detection of the nucleic acid molecule or protein. For example a nucleic acid molecule or an antibody that specifically binds to a molecule can include a label. Examples of labels include, but are not limited to, radioactive isotopes, enzyme substrates, co-factors, ligands, chemiluminescent agents, fluorophores, haptens, enzymes, and combinations thereof. Methods for labeling and guidance in the choice of labels appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998).

Linear Discriminant Function: Discriminant function analysis is used to determine which variables discriminate between two or more naturally occurring groups. Computationally, discriminant function analysis is very similar to analysis of variance (ANOVA). The basic idea underlying discriminant function analysis is to determine whether groups differ with regard to the mean of a variable, and then to use that variable to predict group membership (e.g., of new cases). One can ask whether or not two or more groups are significantly different from each other with respect to the mean of a particular variable. Usually, one includes several variables in a study in order to see which one(s) contribute to the discrimination between groups. In that case, there is a matrix of total variances and covariances; likewise, there is a matrix of pooled within-group variances and covariances. One can compare those two matrices via multivariate F tests in order to determine whether or not there are any significant differences (with regard to all variables) between groups. Step-wise discriminant analysis is a common application of discriminant function analysis is to include many measures in the study, in order to determine the ones that discriminate between groups.

In the two-group case, discriminant function analysis can also be thought of as (and is analogous to) multiple regression (the two-group discriminant analysis is also called Fisher linear discriminant analysis). Another major purpose to which discriminant analysis is applied is the issue of predictive classification of cases. Specific methods for a linear discriminant analysis can be found, for example, on the StatSoft® website (2005).

Nearest centroid method: A statistical method that computes a standardized centroid for each class in the training set. For example, this can be the average gene expression for each gene in each class divided by the within-class standard deviation for that gene. Nearest centroid classification takes the gene expression profile of a new sample, and compares it to each of these class centroids. The class, whose centroid it is closest to, in squared distance, is the predicted class for that new sample. “Nearest shrunken centroid classification” includes a modification to the nearest centroid method. It “shrinks” each of the class centroids toward the overall centroid for all classes by an amount called “the threshold.” This shrinkage consists of moving the centroid towards zero by subtracting the threshold, setting it equal to zero if it hits zero. For example if threshold was 2.0, a centroid of 3.2 would be shrunk to 1.2, a centroid of −3.4 would be shrunk to −1.4, and a centroid of 1.2 would be shrunk to zero. The amount of shrinkage is determined by cross-validation. After shrinking the centroids, the new sample is classified by the usual nearest centroid rule, but using the shrunken class centroids.

The shrinkage has two effects: (1) it can make the classifier more accurate by reducing the effect of noisy genes; (2) it does automatic gene selection for genes that characterize the classes. The use of shrunken centroids to evaluate gene expression is disclosed in Tibshirani et al. (Proc. Natl. Acad. Sci. 99: 6567-72, 2002, incorporated herein by reference). A computer program that evaluates shrunken centroids can be downloaded from the Stanford University department of statistics, Tibshirani homepage, from the internet (available on Jul. 12, 2006).

Normal Tissue: The tissue from an organ of an individual that is not affected by a disease process of interest, such as cancer. Thus, “normal tissue,” with regard to cancer is tissue from an individual who does not have cancer, such as CML. A product, such as protein or mRNA from a “normal tissue pool” is product isolated from at least two subjects not affected by a disease process, such as from subjects who are cancer-free.

Nucleic acid array: An arrangement of nucleic acids (such as DNA or RNA) in assigned locations on a matrix, such as that found in cDNA arrays, or oligonucleotide arrays.

Nucleic acid molecules representing genes: Any nucleic acid, for example DNA (intron or exon or both), cDNA, or RNA (such as mRNA), of any length suitable for use as a probe or other indicator molecule, and that is informative about the corresponding gene.

Nucleotide: Includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide. An “oligonucleotide” is a plurality of joined nucleotides joined by native phosphodiester bonds, between about 6 and about 300 nucleotides in length, for example about 6 to 300 contiguous nucleotides of a nucleic acid molecule encoding a protein of interest. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide.

Particular oligonucleotides and oligonucleotide analogs can include linear sequences up to about 200 nucleotides in length, for example a sequence (such as DNA or RNA) that is at least 6 nucleotides, for example at least 8, at least 10, at least 15, at least 20, at least 21, at least 25, at least 30, at least 35, at least 40, at least 45, at least 50, at least 100 or even at least 200 nucleotides long, or from about 6 to about 50 nucleotides, for example about 10-25 nucleotides, such as 12, 15 or 20 nucleotides. In particular examples, an oligonucleotide includes these numbers of contiguous nucleotides encoding a protein of interest. Such an oligonucleotide can be used on a nucleic acid array or as primers or probes to detect the presence of the nucleic acid molecule encoding the protein of interest.

Oligonucleotide probe: A short sequence of nucleotides, such as at least 8, at least 10, at least 15, at least 20, at least 21, at least 25, or at least 30 nucleotides in length, used to detect the presence of a complementary sequence by molecular hybridization. In particular examples, oligonucleotide probes include a label that permits detection of oligonucleotide probe:target sequence hybridization complexes.

Philadelphia chromosome and BCR-ABL: The Philadelphia chromosome is a specific chromosomal abnormality that is associated with chronic myelogenous leukemia (CML). It is due to a reciprocal translocation designated as t(9;22)(q34;q11), which means an exchange of genetic material between region q34 of chromosome 9 and region q11 of chromosome 22. The presence of this translocation is a highly sensitive test for CML, since 95% of people with CML have this abnormality, while the remainder have either a cryptic translocation that is invisible on G-banded chromosome preparations, or a variant translocation involving another chromosome or chromosomes as well as the long arm of chromosomes 9 and 22).

The result of this translocation is that part of the BCR (“breakpoint cluster region”) gene from chromosome 22 (region q11) is fused with part of the ABL gene on chromosome 9 (region q34). In agreement with the International System for Human Cytogenetic Nomenclature (ISCN), this chromosomal translocation is designated as t(9;22)(q34;q11). ABL stands for “Abelson”, the name of a leukemia virus which carries a similar protein. The result of the translocation is a protein of 210 kDa or 185 kDa. The fused “BCR-ABL” gene is located on the resulting shorter chromosome 22. Because ABL carries a domain that encodes a tyrosine kinase, the BCR-ABL fusion gene is also a tyrosine kinase.

The fused BCR-ABL protein interacts with the interleukin 3beta(c) receptor subunit. The BCR-ABL transcript is constitutively active, i.e. it does not require activation by other cellular messaging proteins. In turn, BCR-ABL activates a number of cell cycle-controlling proteins and enzymes and inhibits DNA repair.

“Complete cytogenetic response” is the effective elimination of the Philadelphia (Ph) chromosome, such that Ph+ metaphases cannot be detected in a biological sample from a subject with CML, such as in CD34+ cells. A major cytogenetic response is when less than 35% Ph+ metaphases can be detected in a sample from the subject.

Prediction Analysis of Microarrays (PAM): A statistical method that used unsupervised hierarchical clustering and evaluate centered correlating distance and average linkage according to the ratios of abundance in each tissue sample as compared with a control, such as a tissue pool, such as from subjects with CML that respond to a BCR-ABL inhibitor. PAM analysis generally utilizes the nearest shrunken centroid classification with 10-fold cross validation. The method is disclosed in Tibshirani et al. (Proc. Natl. Acad. Sci. 99: 6567-72, 2002, incorporated herein by reference). The computer program can be downloaded from the Stanford University department of statistics, Tibshirani homepage on the internet.

Primers: Short nucleic acid molecules, for instance DNA oligonucleotides 10-100 nucleotides in length, such as about 15, 20, 25, 30 or 50 nucleotides or more in length, such as this number of contiguous nucleotides of a nucleotide sequence encoding a protein of interest or other nucleic acid molecule. Primers can be annealed to a complementary target DNA strand by nucleic acid hybridization to form a hybrid between the primer and the target DNA strand. Primer pairs can be used for amplification of a nucleic acid sequence, such as by PCR or other nucleic acid amplification methods known in the art.

Methods for preparing and using nucleic acid primers are described, for example, in Sambrook et al. (In Molecular Cloning: A Laboratory Manual, CSHL, New York, 1989), Ausubel et al. (ed.) (In Current Protocols in Molecular Biology, John Wiley & Sons, New York, 1998), and Innis et al. (PCR Protocols, A Guide to Methods and Applications, Academic Press, Inc., San Diego, Calif., 1990). PCR primer pairs can be derived from a known sequence, for example, by using computer programs intended for that purpose such as Primer (Version 0.5, © 1991, Whitehead Institute for Biomedical Research, Cambridge, Mass.). One of ordinary skill in the art will appreciate that the specificity of a particular primer increases with its length.

In one example, a primer includes at least 15 consecutive nucleotides of a nucleotide molecule, such as at least 18 consecutive nucleotides, at least 20, at least 25, at least 30, at least 35, at least 40, at least 45, at least 50 or more consecutive nucleotides of a nucleotide sequence (such as a gene, mRNA or cDNA). Such primers can be used to amplify a nucleotide sequence of interest encoding a protein, for example using PCR.

Probe: A short sequence of nucleotides, such as at least 8, at least 10, at least 15, at least 20, at least 21, at least 25, or at least 30 nucleotides in length, used to detect the presence of a complementary sequence by molecular hybridization. In particular examples, oligonucleotide probes include a label that permits detection of oligonucleotide probe:target sequence hybridization complexes. For example, an oligonucleotide probe can include these numbers of contiguous nucleotides of a nucleic acid molecule, along with a detectable label. Such an oligonucleotide probe can be used on a nucleic acid array.

Prognosis: The likelihood of the clinical outcome for a subject afflicted with a specific disease or disorder. With regard to cancer, the prognosis is a representation of the likelihood (probability) that the subject will survive (such as for one, two, three, four or five years) and/or the likelihood (probability) that adverse effects will result from the disease. A “poor prognosis” indicates a greater than 50% chance that the subject will not survive to a specified time point (such as one, two, three, for or five years), and/or a greater than 50% chance that the disease will progress, such as the likelihood that a subject with CML will have a blast crises. In several examples, a poor prognosis indicates that there is a greater than 60%, 70%, 80%, or 90% chance that the subject will not survive and/or a greater than 60%, 70%, 80% or 90% chance that the subject will have blast crisis. Conversely, a “good prognosis” indicates a greater than 50% chance that the subject will survive to a specified time point (such as one, two, three, for or five years), and/or a greater than 50% chance that the subject will not have a blast crises. In several examples, a good prognosis indicates that there is a greater than 60%, 70%, 80%, or 90% chance that the subject will survive and/or a greater than 60%, 70%, 80% or 90% chance that the subject will not have a blast crisis.

Purified: The term “purified” does not require absolute purity; rather, it is intended as a relative term. Thus, for example, a purified protein preparation is one in which the protein referred to is more pure than the protein in its natural environment within a cell. For example, a preparation of a protein is purified such that the protein represents at least 50% of the total protein content of the preparation. Similarly, a purified oligonucleotide preparation is one in which the oligonucleotide is more pure than in an environment including a complex mixture of oligonucleotides.

Quantitative real-time PCR (or real time RT-PCR): A method for determining the level of specific DNA or RNA molecules in a biological sample. The accumulation of PCR product is measured at each cycle of a PCR reaction and is compared with a standard curve or quantitated relative to a control DNA or RNA. Quantitative real-time PCR is based on the use of fluorescent dyes or probes to measure the accumulation of PCR product. This may be accomplished through a TAQMAN® assay, where a fluorescently labeled probe is displaced during DNA synthesis by Taq polymerase, resulting in fluorescence, or by inclusion in the PCR reaction of a fluorescent dye such as SYBR® Green, which binds non-specifically to the accumulating double-stranded DNA.

If a standard curve is used to quantitate DNA or RNA, a series of samples containing known amounts of DNA or RNA are run simultaneously with unknown samples. The resulting fluorescence measured from the unknowns may be compared with that from the known samples in order to calculate the quantity of DNA or RNA in the sample. One application of this method is to quantify the expression of an mRNA in one or more samples from subjects.

Quantitative real-time PCR may also be used to determine the relative quantity of a specified RNA present in a sample in comparison to a control sample when knowing the absolute copy number is not necessary. One application of this method is to determine the number of copies of an mRNA in a sample from a subject. The PCR product generated is assessed to determine how many PCR cycles is required from the PCR product to be detectable.

Sample: A biological specimen containing genomic DNA, RNA (including mRNA), protein, cells of interest, or combinations thereof, obtained from a subject. Examples include, but are not limited to, peripheral blood, urine, saliva, tissue biopsy, surgical specimen, and autopsy material. In one example, a sample includes a bone marrow biopsy, or sample of normal tissue (from a subject not afflicted with a known disease or disorder, such as a bone marrow from a cancer-free subject).

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. This homology is more significant when the orthologous proteins or cDNAs are derived from species which are more closely related (such as human and mouse sequences), compared to species more distantly related (such as human and C. elegans sequences).

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site.

BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1154 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15÷20*100=75).

For comparisons of amino acid sequences of greater than about 30 amino acids, the Blast 2 sequences function is employed using the default BLOSUM62 matrix set to default parameters, (gap existence cost of 11, and a per residue gap cost of 1). Homologs are typically characterized by possession of at least 70% sequence identity counted over the full-length alignment with an amino acid sequence using the NCBI Basic Blast 2.0, gapped blastp with databases such as the nr or swissprot database. Queries searched with the blastn program are filtered with DUST (Hancock and Armstrong, 1994, Comput. Appl. Biosci. 10:67-70). Other programs use SEG. In addition, a manual alignment can be performed. Proteins with even greater similarity will show increasing percentage identities when assessed by this method, such as at least about 75%, 80%, 85%, 90%, 95%, 98%, or 99% sequence identity to a gene listed in Table 2.

When aligning short peptides (fewer than around 30 amino acids), the alignment is be performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least about 60%, 70%, 75%, 80%, 85%, 90%, 95%, 98%, 99% sequence identity to a protein encoded by a gene listed in Table 2. When less than the entire sequence is being compared for sequence identity, homologs will typically possess at least 75% sequence identity over short windows of 10-20 amino acids, and can possess sequence identities of at least 85%, 90%, 95% or 98% depending on their identity to the reference sequence. Methods for determining sequence identity over such short windows are described at the NCBI web site.

One indication that two nucleic acid molecules are closely related is that the two molecules hybridize to each other under stringent conditions, as described above. Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. Such homologous nucleic acid sequences can, for example, possess at least about 60%, 70%, 80%, 90%, 95%, 98%, or 99% sequence identity to a nucleic acid of a gene listed in Table 2 is determined by this method. An alternative (and not necessarily cumulative) indication that two nucleic acid sequences are substantially identical is that the polypeptide which the first nucleic acid encodes is immunologically cross reactive with the polypeptide encoded by the second nucleic acid.

One of skill in the art will appreciate that the particular sequence identity ranges are provided for guidance only; it is possible that strongly significant homologs could be obtained that fall outside the ranges provided.

Subject or individual of interest: Living multi-cellular vertebrate organisms, a category that includes human and non-human mammals, such as veterinary subjects. In a particular example, a subject is a human individual who has CML.

Therapeutically effective amount: An amount of a pharmaceutical preparation that alone, or together with a pharmaceutically acceptable carrier or one or more additional therapeutic agents, induces the desired response. A therapeutic agent, such as a BCR-ABL inhibitor, is administered in therapeutically effective amounts.

Therapeutic agents can be administered in a single dose, or in several doses, for example daily, during a course of treatment. However, the effective amount of can be dependent on the source applied, the subject being treated, the severity and type of the condition being treated, and the manner of administration. Effective amounts a therapeutic agent can be determined in many different ways, such as assaying for a sign or a symptom of CML, such as the presence of the Philadelphia chromosome or complete cytogenetic remission. Effective amounts also can be determined through various in vitro, in vivo or in situ assays. For example, a pharmaceutical preparation can decrease one or more symptoms of CML, for example decrease a symptom by at least 20%, at least 50%, at least 70%, at least 90%, at least 98%, or even at least 100%, as compared to an amount in the absence of the pharmaceutical preparation. In one example, a pharmaceutical preparation decreases the number of Ph+ metaphases in a subject with CML.

Treating a disease: “Treatment” refers to a therapeutic intervention that ameliorates a sign or symptom of a disease or pathological condition, such a sign or symptom of CML. Treatment can also induce remission or cure of a condition, or can reduce the pathological condition, or can reduce a sign or symptom, such as the presence of the Philadelphia chromosome. In particular examples, treatment includes preventing a disease, for example by inhibiting the full development of a disease. Treatment of a disease does not require a total absence of disease.

Upregulated or activation: When used in reference to the expression of a nucleic acid molecule, such as a gene, refers to any process which results in an increase in production of a gene product. A gene product can be RNA (such as mRNA, rRNA, tRNA, and structural RNA) or protein. Therefore, gene upregulation or activation includes processes that increase transcription of a gene or translation of mRNA.

Examples of processes that increase transcription include those that facilitate formation of a transcription initiation complex, those that increase transcription initiation rate, those that increase transcription elongation rate, those that increase processivity of transcription and those that relieve transcriptional repression (for example by blocking the binding of a transcriptional repressor). Gene upregulation can include inhibition of repression as well as stimulation of expression above an existing level. Examples of processes that increase translation include those that increase translational initiation, those that increase translational elongation and those that increase mRNA stability.

Gene upregulation includes any detectable increase in the production of a gene product. In certain examples, production of a gene product increases by at least 2-fold, for example at least 3-fold or at least 4-fold, as compared to a control (such an amount of gene expression in a normal cell, or the amount of gene expression in a subject with CML in CCyR). In one example, a control is a centroid value obtained from subjects with CML that have a complete cytogenetic response when treated with a BCR-ABL inhibitor, such as imitinab.

### II. Description of Several Embodiments

Disclosed herein is a gene expression profile that can be used to determine if an individual with CML will achieve a cytogenetic response (such as a complete cytogenetic response CCyR or major cytogenetic response MCyR) in response to treatment with an inhibitor of BCR-ABL, such as imatinib, AMN107 (nilotinib), dasatinib, NS-187, ON012380, Bosutinib (SKI-606), INNO-406 (NS-187), MK-0457 (VX-680), SGX70393 and BMS-354825. This gene signature can be used to determine a subject with CMLs sensitivity to treatment with a BCR-ABL inhibitor, for example, to predict whether a subject will respond to treatment with a BCR-ABL inhibitor, show an initial response but relapse (such as within six months after beginning treatment with a BCR-ABL inhibitor), or will respond positively to treatment with a BCR-ABL inhibitor (for example achieve a MCyR or CCyR with in 24 months, such as within 12 months or within 6 months).

Methods are provided for evaluating a subject with chronic myelogenous leukemia (CML), such as to determine if the subject can be treated with a BCR-ABL inhibitor. For example, the methods disclosed herein can be used to determine the prognosis of the subject, which includes the likelihood (probability) that the subject will respond to treatment with a BCR-ABL inhibitor, or the likelihood (probability) that the subject will have a complete cytogenetic response (CCyR) in response to a therapeutic agent, such as a BCR-ABL inhibitor. In particular examples, the method can determine with a reasonable amount of sensitivity and specificity whether a subject is likely to survive one, two, three, four or five years. In some examples, the gene expression profile can predict response (such as a CCyR) to a BCR-ABL inhibitor with an accuracy of at least about 70% such as with an accuracy of at least about 75%, at least about 80%, at least about 85%, at least about 90%, or at least about 95% (for example, about 71%, about 72%, about 73%, about 74%, about 75%, about 76%, about 77%, about 78%, about 79%, about 80%, about 81%, about 82%, about 83%, about 4%, about 85%, about 86%, about 87%, about 88%, about 89%, about 90%, 91%, about 92%, about 93%, about 94%, about 95%, about 96%, about 97%, about 98%, about 99% or 100%).

In additional examples, the methods include isolating CD34+ cells from the subject, and evaluating gene expression in the isolated CD34+ cells. The CD34+ cells can be all CD34+ cells (CD34+CD38+ and CD34+CD38−) or can be CD34+CD38+ cells or CD34+CD38− cells.

In additional examples, the method is utilized to determine a therapeutic regimen for the subject. In one example, the therapeutic regimen includes treatment with a BCR-ABL inhibitor, such as imatinib, AMN107 (nilotinib), dasatinib, NS-187, ON012380, Bosutinib (SKI-606), INNO-406 (NS-187), MK-0457 (VX-680), SGX70393 and BMS-354825.

In particular examples, the method also includes identifying the subject as being a candidate for treatment with the BCR-ABL inhibitor, and administering a therapeutically effective amount of appropriate BCR-ABL inhibitor. Thus the method can be used to determine if a subject will have a CCyR in response to the BCR-ABL inhibitor. The method can be used to predict if a subject will respond to the BCR-ABL inhibitor, and thus has a good prognosis for survival.

In further examples, the method can identify the subject as not being a candidate for treatment with the BCR-ABL inhibitor. The method identifies the subject as being resistant to treatment with a BCR-ABL inhibitor, so that they will not have a CCyR following treatment with the inhibitor. The method can be used to predict if a subject will not respond to the BCR-ABL inhibitor, and thus has a poor prognosis for survival. Thus, an alternative therapeutic agent can be administered to the subject.

Without being bound by theory, early identification of a subject as resistant to treatment with a BCR-ABL inhibitor, can reduce costs, as costly treatment with an ineffective BCR-ABL inhibitor will not be initiated (or continued). In addition, early identification of a subject as resistant to treatment with a BCR-ABL inhibitor can result in earlier administration of an alternative agent, thus increasing the likelihood of survival and decreasing the likelihood of the subject having a blast crisis.

In particular examples, methods include detecting expression (such as quantitating gene or protein expression) of a plurality of genes of interest in the CD34+ cells from the subject. The genes of interest can include, consist essentially of, or consist of at least five, such as at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65 at least 66, at least 67, or at all 68, such as 5-15, 10-20, 15-25, 20-30, 25-35, 30-40, 35-45, 40-50, 45-55, 50-60, or 55-68 of the genes listed in Table 2 in any combination, such as any combination of at least 5, such as at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65 at least 66, at least 67, or at all 68, such as 5-15, 10-20, 15-25, 20-30, 25-35, 30-40, 35-45, 40-50, 45-55, 50-60, or 55-68 of PHLDB2, GAS2, EGFL6, RXFP1, MMRN1, NGFRAP1L1, SPOCK3, KIF21A, FLJ12033, ANGPT1, TMEM163, EMCN, ITGA2, CLIP4, SH3GL3, SLC8A3, PRKG1, GPRASP2, VWF, BC041986, HEMGN, ZNF44, MEIS1, CMAH, KIAA1598, RP11-145H9.1, RBPMS, MGC1305, NFIB, ARMCX2, ITGB8, CALN1, MPDZ, EVA1, LOH11CR2A, MOSC2, ZNF140, ABAT, C5orf25, KLHL13, MUC4, TPD52L1, TIMP3, BC043173, ZNF253, CEBPB, CECR1, ARL4C, FLJ20273, ADM, AI694722, SLC22A4, AF318321, UPP1, S100A10, P2RY5, IFI30, PTPRE, CLEC7A, SERPINA1, CTSG, SLC16A6, MAFB, MPO, FLJ22662, CSTA, MS4A3, and FCN1.

The method can include identifying an increase or a decrease in the expression of these genes as compared the expression of these genes in CD34+ cells isolated from a subject without CML, or as compared to the expression of these genes in CD34+ cells isolated from a subject with CML who is known to respond to the BCR-ABL inhibitor, such as a subject with a CCyR in response to the BCR-ABL inhibitor. In one embodiment, the method includes detecting an increase in expression of genes encoding molecules involved in cell adhesion. In another embodiment, the method includes detecting a decrease in the expression of genes encoding molecules involved in apoptosis. In a further embodiment, the method includes detection of an increase in the expression of four genes in the focal adhesion pathway. In an additional embodiment, the method involves detection of an increase in the expression of three genes involved in the ECM-receptor interaction pathway. In yet other embodiments, the method includes detecting changes in the expression of genes involved in complement and coagulation cascades, induction of apoptosis through DR3 and DR4/5 Death Receptors, Regulation of ck1/cdk5 by type 1 glutamate receptors, p53 Signaling Pathway, Inhibition of Matrix Metalloproteinases, Hedgehog signaling, and IL 6 signaling pathway.

“Consists essentially of” in this context indicates that the expression of additional molecules can be evaluated (such as a control), but that these molecules do not include more than five other genes. Thus, in one example, the expression of a control, such as a housekeeping protein or rRNA can be assessed (such as 18S RNA, beta-microglobulin, GAPDH, and/or 18S rRNA). In some examples, “consist essentially of” indicates that no more than 5 other molecules are evaluated, such as no more than 4, 3, 2, or 1 other molecules, such as the expression of housekeeping genes. In this context “consist of” indicates that only the expression of the stated molecules are evaluated; the expression of additional molecules is not evaluated.

In some examples, expression values are compared to a reference value, such as a value representing expression for the same gene in CD34+ cells from an individual with a known CCyR status and prognosis. For example, the resulting difference in expression levels can be represented as differential expression, which can be represented by increased or decreased expression in the at least one gene (for instance, a nucleic acid molecule or a protein). For example, differential expression includes, but is not limited to, an increase or decrease in an amount of a nucleic acid molecule or protein, the stability of a nucleic acid molecule or protein, the localization of a nucleic acid molecule or protein, or the biological activity of a nucleic acid molecule or protein. In some examples, the method also includes detecting expression (such as quantitating gene or protein expression) of a plurality of genes of interest in CD34+ cells isolated from subjects that do not have CML (“cancer-free” individuals). In additional embodiments, the control is the quantitative or qualitative expression of the gene in CD34+ cells from a subject with CML that is responding to the BCR-ABL inhibitor, such as a subject with a CCyR. In further examples, the control is a set of standard values that correspond to the average gene expression in CD34+ cells from a population of subjects that do not have CML, or a population of subject that all response to the BCR-ABL inhibitor.

Specific examples include evaluative methods in which changes in gene expression of least five, such as at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65 at least 66, at least 67, or at all 68, such as 5-15, 10-20, 15-25, 20-30, 25-35, 30-40, 35-45, 40-50, 45-55, 50-60, or 55-68 of the genes listed in Table 2 in CD34+ cells are determined.

For example, real time RT-PCR can be used to quantitate mRNA expression. However, one skilled in the art will appreciate that other methods can be used to detect expression, such as other nucleic acid molecule detection methods, or protein expression can be determined. Such methods are routine in the art. The obtained raw data can be used directly, or normalized to a control. Exemplary controls include a reference value or range of values representing expression of the gene in normal CD34+ cells, or in CD34+ cells from a subject in CCyR. As such, the expression of least five, such as at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65 at least 66, at least 67, or at all 68, such as 5-15, 10-20, 15-25, 20-30, 25-35, 30-40, 35-45, 40-50, 45-55, 50-60, or 55-68 of the genes listed in Table 2 can also be evaluated in normal CD34+ cells, such as a pool of samples of CD34+ cells from individuals that do not have CML. In such an example, the raw data for each gene product (or control) is normalized to the appropriate gene (or control) reference value for the normal tissue, and this normalized value used for further analysis. In a particular example, the gene expression data (raw or normalized) from CD34+ cells from a subject with CML in CCyR that responds to the BCR-ABL inhibitor, as well as the appropriate classification tables, are inputted, for example into a algorithm that can generate class centroids from the classification table.

The classification tables are subjected to the algorithm for “training”, which provides a type of calibration to generate centroids for each gene and each classification (responder, non-responder, good prognosis, poor prognosis). This provides a classification for responder/non-responder and good/poor prognosis for known conditions, which can be used to then classify a subject of interest with an unknown prognosis and unknown ability to respond to the BCR-ABL inhibitor. The algorithm then compares the values for the subject of interest using distance between the sample and the class centroids, and outputs a responder or non-responder status, as well a prognosis. The algorithm also compares the test sample gene expression values to known values using distance between the sample and the class centroids. The sample is then classified as a non-responder or responder and good prognosis or poor prognosis, for example by using the class centroid closest to the expression profile of the sample. Based on the responder status and prognosis status determined, the subject can be classified as low risk or high risk of death, for example the likelihood of death within one year, three years, or five years, and/or can be classified as low or high risk of blast crisis, such as likelihood of a blast crisis in one year, three years or five years. An exemplary algorithm that can be used is prediction analysis of microarrays (PAM). The method is described, for example, in Tibshirani et al., Proc. Nat. Acad. Sci. 99:6567-62, 2002, incorporated by reference herein in its entirety.

### A. Evaluating Nucleic Acid

Gene expression can be evaluated by detecting mRNA transcribed from a gene of interest in CD34+ cells, or cDNA transcribed from such mRNA thereby detecting the mRNA indirectly. Thus, the disclosed methods can include evaluating mRNA encoding at least five, such as at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65 at least 66, at least 67, or at all 68, such as 5-15, 10-20, 15-25, 20-30, 25-35, 30-40, 35-45, 40-50, 45-55, 50-60, or 55-68 of the genes listed in Table 2. In some examples, the mRNA or cDNA is quantitated.

RNA can be isolated from a sample of CD34+ cells isolated from a subject of interest with CML, CD34+ cells isolated from a normal subject, or CD34+ cells isolated from a subject with CML that has been treated with a BCR-ABL inhibitor and is in CCyR, using methods well known to one skilled in the art, including commercially available kits. General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andres et al., BioTechniques 18: 42044 (1995). In one example, RNA isolation can be performed using purification kit, buffer set and protease from commercial manufacturers, such as QIAGEN®, according to the manufacturer's instructions. For example, total RNA from cells in culture (such as those obtained from a subject) can be isolated using QIAGIN® RNeasy mini-columns. Other commercially available RNA isolation kits include MASTERPURE®. Complete DNA and RNA Purification Kit (EPICENTRE® Madison, Wis.), and Paraffin Block RNA Isolation Kit (Ambion®, Inc.). Total RNA from tissue samples can be isolated using RNA Stat-60 (Tel-Test). RNA prepared from tumor or other biological sample can be isolated, for example, by cesium chloride density gradient centrifugation.

Methods of gene expression profiling include methods based on hybridization analysis of polynucleotides, methods based on sequencing of polynucleotides, and other genomics-based methods. In some examples, mRNA expression in a sample is quantified using northern blotting or in situ hybridization (Parker & Barnes, Methods in Molecular Biology 106:247-283, 1999); RNAse protection assays (Hod, Biotechniques 13:852-4, 1992); and PCR-based methods, such as reverse transcription polymerase chain reaction (RT-PCR) (Weis et al., Trends in Genetics 8:263-4, 1992). Alternatively, antibodies can be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS). In one example, RT-PCR can be used to compare mRNA levels in different samples, in normal and tumor tissues, with or without drug treatment, to characterize patterns of gene expression, to discriminate between closely related mRNAs, and to analyze RNA structure.

Methods for quantitating mRNA are well known in the art. In one example, the method utilizes RT-PCR. Generally, the first step in gene expression profiling by RT-PCR is the reverse transcription of the RNA template into cDNA, followed by its exponential amplification in a PCR reaction. Two commonly used reverse transcriptases are avian myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif, USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. TaqMan®PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TAQMAN® RT-PCR can be performed using commercially available equipment, such as, for example, ABI PRISM 7700® Sequence Detection System® (Perkin-Elmer-Applied Biosystems, Foster City, Calif.), or Lightcycler (Roche Molecular Biochemicals, Mannheim, Germany). In one example, the 5′ nuclease procedure is run on a real-time quantitative PCR device such as the ABI PRISM 7700® Sequence Detection System®. The system includes of thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

In some examples, 5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is can be performed using an internal standard. The ideal internal standard is expressed at a constant level among different tissues, and is unaffected by the experimental treatment. RNAs commonly used to normalize patterns of gene expression are mRNAs for the housekeeping genes glyceraldehyde-3-phosphate-dehydrogenase (GAPDH), beta-actin, and 18S ribosomal RNA.

A variation of RT-PCR is real time quantitative RT-PCR, which measures PCR product accumulation through a dual-labeled fluorigenic probe (e.g. TAQMAN® probe). Real time PCR is compatible both with quantitative competitive PCR, where internal competitor for each target sequence is used for normalization, and with quantitative comparative PCR using a normalization gene contained within the sample, or a housekeeping gene for RT-PCR (see Held et al., Genome Research 6:986 994, 1996). Quantitative PCR is also described in U.S. Pat. No. 5,538,848. Related probes and quantitative amplification procedures are described in U.S. Pat. No. 5,716,784 and U.S. Pat. No. 5,723,591. Instruments for carrying out quantitative PCR in microtiter plates are available from PE Applied Biosystems, 850 Lincoln Centre Drive, Foster City, Calif. 94404 under the trademark ABI PRISM® 7700.

The steps of a representative protocol for quantitating gene expression using fixed, paraffin-embedded tissues, such as bone marrow as the RNA source, including mRNA isolation, purification, primer extension and amplification are given in various published journal articles (see Godfrey et al., J. Mol. Diag. 2:84 91, 2000; Specht et al., Am. J. Pathol. 158:419-29, 2001). Briefly, a representative process starts with cutting about 10 μm thick sections of paraffin-embedded tumor tissue samples or adjacent non-cancerous tissue. The RNA is then extracted, and protein and DNA are removed. Alternatively, RNA is located directly from a sample, such as a population of CD34+ cells. After analysis of the RNA concentration, RNA repair and/or amplification steps can be included, if necessary, and RNA is reverse transcribed using gene specific promoters followed by RT-PCR.

The primers used for the amplification are selected so as to amplify a unique segment of the gene of interest, such as mRNA encoding at least five, such as at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65 at least 66, at least 67, or at all 68, such as 5-15, 10-20, 15-25, 20-30, 25-35, 30-40, 35-45, 40-50, 45-55, 50-60, or 55-68 of the genes listed in Table 2.

An alternative quantitative nucleic acid amplification procedure is described in U.S. Pat. No. 5,219,727. In this procedure, the amount of a target sequence in a sample is determined by simultaneously amplifying the target sequence and an internal standard nucleic acid segment. The amount of amplified DNA from each segment is determined and compared to a standard curve to determine the amount of the target nucleic acid segment that was present in the sample prior to amplification.

As discussed above, in some embodiments of this method, the expression of a “house keeping” gene or “internal control” can also be evaluated. These terms include any constitutively or globally expressed gene whose presence enables an assessment of mRNA levels of genes of interest. Such an assessment includes a determination of the overall constitutive level of gene transcription and a control for variations in RNA recovery.

In some examples, gene expression is identified or confirmed using the microarray technique. Thus, the expression profile can be measured in either fresh or paraffin-embedded tissue, using microarray technology. In this method, nucleic acid sequences of interest (including cDNAs and oligonucleotides) are plated, or arrayed, on a microchip substrate. The arrayed sequences are then hybridized with specific DNA probes from cells or tissues of interest. Just as in the RT-PCR method, the source of mRNA typically is total RNA isolated from human tumors, and corresponding noncancerous tissue and normal tissues or cell lines.

In a specific embodiment of the microarray technique, PCR amplified inserts of cDNA clones are applied to a substrate in a dense array. Probes for at least five, such as at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65 at least 66, at least 67, or at all 68, such as 5-15, 10-20, 15-25, 20-30, 25-35, 30-40, 35-45, 40-50, 45-55, 50-60, or 55-68 of nucleotide sequences encoding the genes listed in Table 2 are applied to the substrate, and the array can consist essentially of, or consist of these sequences. The microarrayed nucleic acids are suitable for hybridization under stringent conditions.

Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for at least five, such as at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65 at least 66, at least 67, or at all 68, such as 5-15, 10-20, 15-25, 20-30, 25-35, 30-40, 35-45, 40-50, 45-55, 50-60, or 55-68 of the genes listed in Table 2. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93(2):10614-9, 1996). Microarray analysis can be performed by commercially available equipment, following manufacturer's protocols, such as are supplied with Affymetrix® GenChip® technology, or Incyte's microarray technology.

Serial analysis of gene expression (SAGE) is another method that allows the simultaneous and quantitative analysis of a large number of gene transcripts, without the need of providing an individual hybridization probe for each transcript. First, a short sequence tag (about 10-14 base pairs) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, for example, Velculescu et al., Science 270:484-7, 1995; and Velculescu et al., Cell 88:243-51, 1997.

### B. Evaluation of Proteins

In some examples, expression of the proteins encoded by at least five, such as at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65 at least 66, at least 67, or at all 68, such as 5-15, 10-20, 15-25, 20-30, 25-35, 30-40, 35-45, 40-50, 45-55, 50-60, or 55-68 the genes listed in Table 2, such as by at least five, such as at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65 at least 66, at least 67, or at all 68, such as 5-15, 10-20, 15-25, 20-30, 25-35, 30-40, 35-45, 40-50, 45-55, 50-60, or 55-68 of PHLDB2, GAS2, EGFL6, RXFP1, MMRN1, NGFRAP1L1, SPOCK3, KIF21A, FLJ12033, ANGPT1, TMEM163, EMCN, ITGA2, CLIP4, SH3GL3, SLC8A3, PRKG1, GPRASP2, VWF, BC041986, HEMGN, ZNF44, MEIS1, CMAH, KIAA1598, RP11-145H9.1, RBPMS, MGC1305, NFIB, ARMCX2, ITGB8, CALN1, MPDZ, EVA1, LOH11CR2A, MOSC2, ZNF140, ABAT, C5orf25, KLHL13, MUC4, TPD52L1, TIMP3, BC043173, ZNF253, CEBPB, CECR1, ARL4C, FLJ20273, ADM, AI694722, SLC22A4, AF318321, UPP1, S100A10, P2RY5, IFI30, PTPRE, CLEC7A, SERPINA1, CTSG, SLC16A6, MAFB, MPO, FLJ22662, CSTA, MS4A3, and FCN1 are analyzed.

Suitable biological samples include samples containing protein obtained from CD34+ cells from a subject of interest, CD34+ cells a subject without CML, and CD34+ cells from a subject with CML who has been treated with a BCR-ABL inhibitor and is in CCyR. An alteration in the amount of the proteins encoded by at least five, such as at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65 at least 66, at least 67, or at all 68, such as 5-15, 10-20, 15-25, 20-30, 25-35, 30-40, 35-45, 40-50, 45-55, 50-60, or 55-68 of the genes listed in Table 2 in CD34+ cells isolated from the subject of interest with CML, such as an increase or decrease in expression, indicates the prognosis of the subject, or the susceptibility of the subject to treatment with the BCR-ABL inhibitor, as described above.

The availability of antibodies specific to proteins encoded by at least five, such as at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65 at least 66, at least 67, or at all 68, such as 5-15, 10-20, 15-25, 20-30, 25-35, 30-40, 35-45, 40-50, 45-55, 50-60, or 55-68 68 of the genes listed in Table 2 in facilitates the detection and quantitation of these proteins by one of a number of immunoassay methods that are well known in the art, such as those presented in Harlow and Lane (Antibodies, A Laboratory Manual, CSHL, New York, 1988). Methods of producing antibodies are also known in the art.

Any standard immunoassay format (such as ELISA, Western blot, or RIA assay) can be used to measure protein levels. Thus, the level of at least five, such as at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65 at least 66, at least 67, or at all 68, such as 5-15, 10-20, 15-25, 20-30, 25-35, 30-40, 35-45, 40-50, 45-55, 50-60, or 55-68 of 68 of the genes listed in Table 2 in isolated CD34+ cells can be evaluated using these methods.

Immunohistochemical techniques can also be utilized for detection and quantification. General guidance regarding such techniques can be found in Bancroft and Stevens (Theory and Practice of Histological Techniques, Churchill Livingstone, 1982) and Ausubel et al. (Current Protocols in Molecular Biology, John Wiley & Sons, New York, 1998). Quantitation of the protein encoded by any of the genes listed in Table 2, such as PHLDB2, GAS2, EGFL6, RXFP1, MMRN1, NGFRAP1L1, SPOCK3, KIF21A, FLJ12033, ANGPT1, TMEM163, EMCN, ITGA2, CLIP4, SH3GL3, SLC8A3, PRKG1, GPRASP2, VWF, BC041986, HEMGN, ZNF44, MEIS1, CMAH, KIAA1598, RP11-145H9.1, RBPMS, MGC1305, NFIB, ARMCX2, ITGB8, CALN1, MPDZ, EVA1, LOH11CR2A, MOSC2, ZNF140, ABAT, C5orf25, KLHL13, MUC4, TPD52L1, TIMP3, BC043173, ZNF253, CEBPB, CECR1, ARL4C, FLJ20273, ADM, AI694722, SLC22A4, AF318321, UPP1, S100A10, P2RY5, IFI30, PTPRE, CLEC7A, SERPINA1, CTSG, SLC16A6, MAFB, MPO, FLJ22662, CSTA, MS4A3, and FCN1 can be achieved by immunoassay. The amounts of these proteins in the CD34+ cells isolated from the subject of interest, CD34+ cells isolated from a subject with CML who has been treated with a BCR-ABL inhibitor and is in CCyR, and/or CD34+ cells isolated from a subject without CCyR can be compared. A significant increase or decrease in the amount can be evaluated using statistical methods disclosed herein and/or known in the art.

Quantitative spectroscopic approaches methods, such as SELDI, can be used to analyzed the presence of the protein encoded by the genes listed in Table 2. In one example, surface-enhanced laser desorption-ionization time-of-flight (SELDI-TOF) mass spectrometry is used to detect protein expression, for example by using the ProteinChip™ (Ciphergen Biosystems, Palo Alto, Calif.). Such methods are well known in the art (for example see U.S. Pat. No. 5,719,060; U.S. Pat. No. 6,897,072; and U.S. Pat. No. 6,881,586). SELDI is a solid phase method for desorption in which the analyte is presented to the energy stream on a surface that enhances analyte capture or desorption.

Briefly, one version of SELDI uses a chromatographic surface with a chemistry that selectively captures analytes of interest, such as proteins encoded by genes listed in Table 2. Chromatographic surfaces can be composed of hydrophobic, hydrophilic, ion exchange, immobilized metal, or other chemistries. For example, the surface chemistry can include binding functionalities based on oxygen-dependent, carbon-dependent, sulfur-dependent, and/or nitrogen-dependent means of covalent or noncovalent immobilization of analytes. The activated surfaces are used to covalently immobilize specific “bait” molecules such as antibodies, receptors, or oligonucleotides often used for biomolecular interaction studies such as protein-protein and protein-DNA interactions.

The surface chemistry allows the bound analytes to be retained and unbound materials to be washed away. Subsequently, analytes bound to the surface can be desorbed and analyzed by any of several means, for example using mass spectrometry. When the analyte is ionized in the process of desorption, such as in laser desorption/ionization mass spectrometry, the detector can be an ion detector. Mass spectrometers generally include means for determining the time-of-flight of desorbed ions. This information is converted to mass. However, one need not determine the mass of desorbed ions to resolve and detect them: the fact that ionized analytes strike the detector at different times provides detection and resolution of them. Alternatively, the analyte can be detectably labeled (for example with a fluorophore or radioactive isotope). In these cases, the detector can be a fluorescence or radioactivity detector. A plurality of detection means can be implemented in series to fully interrogate the analyte components and function associated with retained molecules at each location in the array.

Therefore, in a particular example, the chromatographic surface includes antibodies that specifically bind the proteins encoded by at least five, such as at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65 at least 66, at least 67, or at all 68, such as 5-15, 10-20, 15-25, 20-30, 25-35, 30-40, 35-45, 40-50, 45-55, 50-60, or 55-68 of the genes listed in Table 2, such as PHLDB2, GAS2, EGFL6, RXFP1, MMRN1, NGFRAP1L1, SPOCK3, KIF21A, FLJ12033, ANGPT1, TMEM163, EMCN, ITGA2, CLIP4, SH3GL3, SLC8A3, PRKG1, GPRASP2, VWF, BC041986, HEMGN, ZNF44, MEIS1, CMAH, KIAA1598, RP11-145H9.1, RBPMS, MGC1305, NFIB, ARMCX2, ITGB8, CALN1, MPDZ, EVA1, LOH11CR2A, MOSC2, ZNF140, ABAT, C5orf25, KLHL13, MUC4, TPD52L1, TIMP3, BC043173, ZNF253, CEBPB, CECR1, ARL4C, FLJ20273, ADM, AI694722, SLC22A4, AF318321, UPP1, S100A10, P2RY5, IFI30, PTPRE, CLEC7A, SERPINA1, CTSG, SLC16A6, MAFB, MPO, FLJ22662, CSTA, MS4A3, and FCN1. In other examples, the chromatographic surface consists essentially of, or consists of, antibodies that specifically bind at least five, such as at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65 at least 66, at least 67, or at all 68, such as 5-15, 10-20, 15-25, 20-30, 25-35, 30-40, 35-45, 40-50, 45-55, 50-60, or 55-68 Table 2. In this context “consists essentially of” indicates that the chromatographic surface does not include more than five, more than four, more than three, more than four, but can include antibodies that bind other molecules, such as housekeeping proteins (e.g. actin or myosin).

In another example, antibodies are immobilized onto the surface using a bacterial Fc binding support. The chromatographic surface is incubated with a sample. The antigens present in the sample can recognize the antibodies on the chromatographic surface. The unbound proteins and mass spectrometric interfering compounds are washed away and the proteins that are retained on the chromatographic surface are analyzed and detected by SELDI-TOF. The MS profile from the sample can be then compared using differential protein expression mapping, whereby relative expression levels of proteins at specific molecular weights are compared by a variety of statistical techniques and bioinformatic software systems. It should be noted that these values can also be inputted into PAM.

In other examples the antibody that specifically binds a protein encoded by a gene listed in Table 2 is directly labeled with a detectable label. In another example, each antibody that specifically binds a protein encoded by a gene listed in Table 2 is unlabeled and a second antibody or other molecule that can bind the first antibody that specifically binds the protein encoded by a gene listed in Table 2 is labeled. As is well known to one of skill in the art, a second antibody is chosen that is able to specifically bind the specific species and class of the first antibody. For example, if the first antibody is a human IgG, then the secondary antibody can be an anti-human-IgG. Other molecules that can bind to antibodies include, without limitation, Protein A and Protein G, both of which are available commercially.

Suitable labels for the antibody or secondary antibody include various enzymes, prosthetic groups, fluorescent materials, luminescent materials, magnetic agents and radioactive materials. Non-limiting examples of suitable enzymes include horseradish peroxidase, alkaline phosphatase, beta-galactosidase, or acetylcholinesterase. Non-limiting examples of suitable prosthetic group complexes include streptavidin/biotin and avidin/biotin. Non-limiting examples of suitable fluorescent materials include umbelliferone, fluorescein, fluorescein isothiocyanate, rhodamine, dichlorotriazinylamine fluorescein, dansyl chloride or phycoerythrin. A non-limiting exemplary luminescent material is luminol; a non-limiting exemplary magnetic agent is gadolinium, and non-limiting exemplary radioactive labels include ¹²⁵I, ¹³¹I, ³⁵S or ³H.

In an alternative example, proteins encoded by the genes listed in Table 2 can be assayed in a biological sample by a competition immunoassay utilizing standards of a protein encoded by a gene listed in Table 2 labeled with a detectable substance and an unlabeled antibody that specifically binds the desired protein encoded by a gene listed in Table 2. In this assay, the sample and the labeled standards and the antibody that specifically binds the desired protein encoded by a gene listed in Table 2 are combined and the amount of labeled standard bound to the unlabeled antibody is determined. The amount of protein encoded by a gene listed in Table 2 in the biological sample is inversely proportional to the amount of labeled standard bound to the antibody that specifically binds the protein encoded by a gene listed in Table 2.

### C. Arrays

Arrays are disclosed herein that include oligonucleotide probes consisting essentially of, or consisting of at least five, such as at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65 at least 66, at least 67, or at all 68, such as 5-15, 10-20, 15-25, 20-30, 25-35, 30-40, 35-45, 40-50, 45-55, 50-60, or 55-68 of the nucleic acid sequences of the genes listed in table 2.

The methods and apparatus in accordance with the present disclosure takes advantage of the fact that under appropriate conditions oligonucleotides form base-paired duplexes with nucleic acid molecules that have a complementary base sequence. The stability of the duplex is dependent on a number of factors, including the length of the oligonucleotides, the base composition, and the composition of the solution in which hybridization is effected. The effects of base composition on duplex stability can be reduced by carrying out the hybridization in particular solutions, for example in the presence of high concentrations of tertiary or quaternary amines.

The thermal stability of the duplex is also dependent on the degree of sequence similarity between the sequences. By carrying out the hybridization at temperatures close to the anticipated Tₘ's of the type of duplexes expected to be formed between the target sequences and the oligonucleotides bound to the array, the rate of formation of mis-matched duplexes can be substantially reduced.

The length of each oligonucleotide sequence employed in the array can be selected to optimize binding to an mRNA. An optimum length for use with a particular marker nucleic acid sequence under specific screening conditions can be determined empirically. Thus, the length for each individual element of the set of oligonucleotide sequences included in the array can be optimized for screening. In one example, oligonucleotide probes are from about 20 to about 35 nucleotides in length or about 25 to about 40 nucleotides in length.

The oligonucleotide probe sequences forming the array can be directly linked to the support, for example via the 5′- or 3′-end of the probe. In one example, the oligonucleotides are bound to the solid support by the 5′ end. However, one of skill in the art can determine whether the use of the 3′ end or the 5′ end of the oligonucleotide is suitable for bonding to the solid support. In general, the internal complementarity of an oligonucleotide probe in the region of the 3′ end and the 5′ end determines binding to the support. Alternatively, the oligonucleotide probes can be attached to the support by sequences such as oligonucleotides or other molecules that serve as spacers or linkers to the solid support.

In particular examples, the array is a microarray formed from glass (silicon dioxide). Suitable silicon dioxide types for the solid support include, but are not limited to: aluminosilicate, borosilicate, silica, soda lime, zinc titania and fused silica (for example see Schena, Micraoarray Analysis. John Wiley & Sons, Inc, Hoboken, N.J., 2003). The attachment of nucleic acids to the surface of the glass can be achieved by methods known in the art, for example by surface treatments that form from an organic polymer. Particular examples include, but are not limited to: polypropylene, polyethylene, polybutylene, polyisobutylene, polybutadiene, polyisoprene, polyvinylpyrrolidine, polytetrafluroethylene, polyvinylidene difluroide, polyfluoroethylene-propylene, polyethylenevinyl alcohol, polymethylpentene, polycholorotrifluoroethylene, polysulformes, hydroxylated biaxially oriented polypropylene, aminated biaxially oriented polypropylene, thiolated biaxially oriented polypropylene, etyleneacrylic acid, ethylene methacrylic acid, and blends of copolymers thereof (see U.S. Pat. No. 5,985,567), organosilane compounds that provide chemically active amine or aldehyde groups, epoxy or polylysine treatment of the microarray. Another example of a solid support surface is polypropylene.

In general, suitable characteristics of the material that can be used to form the solid support surface include: being amenable to surface activation such that upon activation, the surface of the support is capable of covalently attaching a biomolecule such as an oligonucleotide thereto; amenability to “in situ” synthesis of biomolecules; being chemically inert such that at the areas on the support not occupied by the oligonucleotides are not amenable to non-specific binding, or when non-specific binding occurs, such materials can be readily removed from the surface without removing the oligonucleotides.

In one example, the surface treatment is amine-containing silane derivatives. Attachment of nucleic acids to an amine surface occurs via interactions between negatively charged phosphate groups on the DNA backbone and positively charged amino groups (Schena, Micraoarray Analysis. John Wiley & Sons, Inc, Hoboken, N.J., 2003). In another example, reactive aldehyde groups are used as surface treatment. Attachment to the aldehyde surface is achieved by the addition of 5′-amine group or amino linker to the DNA of interest. Binding occurs when the nonbonding electron pair on the amine linker acts as a nucleophile that attacks the electropositive carbon atom of the aldehyde group.

A wide variety of array formats can be employed in accordance with the present disclosure. One example includes a linear array of oligonucleotide bands, generally referred to in the art as a dipstick. Another suitable format includes a two-dimensional pattern of discrete cells (such as 4096 squares in a 64 by 64 array). As is appreciated by those skilled in the art, other array formats including, but not limited to slot (rectangular) and circular arrays are equally suitable for use (see U.S. Pat. No. 5,981,185). In one example, the array is formed on a polymer medium, which is a thread, membrane or film. An example of an organic polymer medium is a polypropylene sheet having a thickness on the order of about 1 mil. (0.001 inch) to about 20 mil., although the thickness of the film is not critical and can be varied over a fairly broad range. Biaxially oriented polypropylene (BOPP) films are also suitable in this regard; in addition to their durability, BOPP films exhibit a low background fluorescence. In a particular example, the array is a solid phase, Allele-Specific Oligonucleotides (ASO) based nucleic acid array.

The array formats of the present disclosure can be included in a variety of different types of formats. A “format” includes any format to which the solid support can be affixed, such as microtiter plates, test tubes, inorganic sheets, dipsticks, and the like. For example, when the solid support is a polypropylene thread, one or more polypropylene threads can be affixed to a plastic dipstick-type device; polypropylene membranes can be affixed to glass slides. The particular format is, in and of itself, unimportant. All that is necessary is that the solid support can be affixed thereto without affecting the functional behavior of the solid support or any biopolymer absorbed thereon, and that the format (such as the dipstick or slide) is stable to any materials into which the device is introduced (such as clinical samples and hybridization solutions).

The arrays of the present disclosure can be prepared by a variety of approaches. In one example, oligonucleotide or protein sequences are synthesized separately and then attached to a solid support (see U.S. Pat. No. 6,013,789). In another example, sequences are synthesized directly onto the support to provide the desired array (see U.S. Pat. No. 5,554,501). Suitable methods for covalently coupling oligonucleotides and proteins to a solid support and for directly synthesizing the oligonucleotides or proteins onto the support are known to those working in the field; a summary of suitable methods can be found in Matson et al., Anal. Biochem. 217:306-10, 1994. In one example, the oligonucleotides are synthesized onto the support using conventional chemical techniques for preparing oligonucleotides on solid supports (such as see PCT Publication No. WO 85/01051 and PCT Publication No. WO 89/10977, or U.S. Pat. No. 5,554,501).

A suitable array can be produced using automated means to synthesize oligonucleotides in the cells of the array by laying down the precursors for the four bases in a predetermined pattern. Briefly, a multiple-channel automated chemical delivery system is employed to create oligonucleotide probe populations in parallel rows (corresponding in number to the number of channels in the delivery system) across the substrate. Following completion of oligonucleotide synthesis in a first direction, the substrate can then be rotated by 90° to permit synthesis to proceed within a second)(2° set of rows that are now perpendicular to the first set. This process creates a multiple-channel array whose intersection generates a plurality of discrete cells.

In particular examples, the oligonucleotide probes on the array include one or more labels, which permit detection of oligonucleotide probe:target sequence hybridization complexes.

The disclosure is illustrated by the following non-limiting Examples.

### EXAMPLES

### Example 1

### BCR-ABL in Patients with Primary Cytogenetic Response (CCyR)

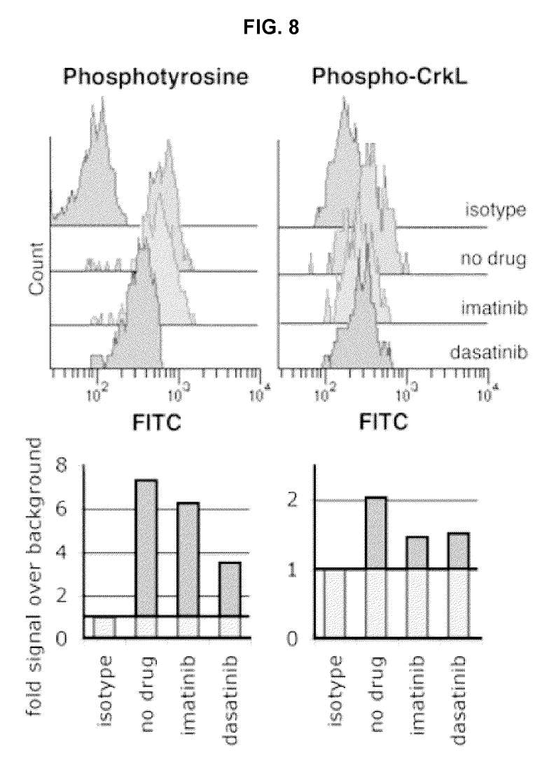

To study whether BCR-ABL is inhibited or active in cells from patients with primary cytogenetic resistance to imatinib a FACS assays was optimized to accurately measure total cellular phosphotyrosine and phospho-CrkL levels in cells treated ex vivo with imatinib or dasatinib. In several patients, both drugs inhibited CrkL phosphorylation to a similar extent, consistent with suppression of BCR-ABL signaling. In contrast, total phosphotyrosine levels were only mildly reduced in the presence of imatinib, but significantly with dasatinib (FIG. 8). BCR-ABL sequencing was negative for kinase domain mutations. This suggests that in these patients leukemia cells have become independent of BCR-ABL through activation of a dasatinib-sensitive but imatinib-resistant pathway. Thus, detecting resistance to a BCR-ABL inhibitor can be useful to initiate therapy with another agent.

### Example 2

### Transcriptosomal Profile

Based on the hypothesis that cytogenetic refractoriness may be a property of leukemic progenitor rather than differentiated cells, gene expression profiling of CD34+ cells was evaluated as a tool for predicting CCyR. Two independent data sets were generated to allow development of the classifier. On the validation set, the classifier had an estimated accuracy rate of 86.9%. Examination of functional annotation for the transcripts in the classifier identified several functional clusters that are highly correlated with respect to direction of response (e.g. transcription factors) and may drive the biology of cytogenetic refractoriness.

### Methods:

Patients: The training set was retrospectively selected from CML patients treated at Oregon Health and Science University (OHSU) between 1998 and 2004. Most of the patients had failed prior interferon-α-based therapy and were treated on phase 2 studies of imatinib prior to its regulatory approval. Eligibility criteria were a diagnosis of CML in chronic phase, availability of bone marrow (BM) mononuclear cells (MNC) stored immediately prior to initiating imatinib therapy and availability of at least 1-year follow-up, including karyotyping. To optimize the chances of detecting differences between responders and non-responders, the study was focused on patients with complete cytogenetic response (CCyR) during their first year of imatinib therapy as opposed to patients who had not achieved even a minor cytogenetic response (i.e. remained at least 66% Ph+) during that time, thus enriching the training set for the extremes of the response spectrum. Fifty-one patients met these criteria. The second group of patients (validation set) consisted of 23 consecutive newly diagnosed chronic phase patients treated with imatinib at the University of Newcastle (United Kingdom) or Leipzig (Germany). In these patients CD34+ cells were selected from peripheral blood collected at diagnosis. All subjects provided written informed consent in accordance with the Declaration of Helsinki.

Data Sets: Two independent data sets were generated. The first data set (learning set) was based on patients with CML who had either achieved a complete cytogenetic response (CCyR) within 1 year of imatinib therapy (R, n=24), or remained at least 65% Ph+ (NR, n=12). The prospectively collected, completely independent validation data set was based on 23 additional subjects using the same criteria (17 R and 6 NR).

Isolation of CD34+ cells: In the case of the training set CD34+ cells were isolated from cryopreserved MNC using a multistep procedure, involving immunomagnetic columns to remove dead cells and fluorescence activated cell sorting (FACS) for CD34+ cell selection. RNA lysates were prepared and stored at −800 until further processed. FISH for BCR-ABL was performed on sorted cells using a commercial probe set (Vysis, Downer's Grove, Ill.). In the case of the validation set CD34+ cells were separated from freshly isolated MNC using MiniMACS columns (Miltenyi Biotec, Bergisch-Gladbach, Germany), following the instructions of the manufacturer. After isolation RNA lysates were prepared and stored following same protocol as for the training set. In the case of the training set MNC had been purified from BM by density gradient centrifugation and cryopreserved in liquid nitrogen. Immediately prior to CD34+ cell extraction, the cells were thawed at 37° C. and washed in Dulbecco's phosphate buffered saline (PBS) containing 0.1% human albumin (Baxter Healthcare Corporation, Glendale, Calif.), 1% recombinant DNase (Pulmozyme™, Genentech, San Francisco, Calif.) and 2.5 mM MgCl2. The samples were enriched for viable cells using the Dead Cell Removal Kit (Miltenyi Biotec, Auburn, Calif.). Next, the cells were resuspended in Hanks' balanced salt solution (HBSS) with 0.5% fetal bovine serum (FBS), 2% HEPES and 1% recombinant human DNase (Genentech), stained with CD34-fluorescein isothiocyanate (FITC) and CD45-PerCP-Cy5.5 monoclonal antibodies (BD Biosciences, San Jose, Calif.), and placed in HBSS containing 0.5% FBS, 2% HEPES and 1% recombinant human DNase. For the identification of dead cells, propidium iodide (PI) (Roche, Indianapolis, Ind.) was added to the cell solution immediately prior to sorting.

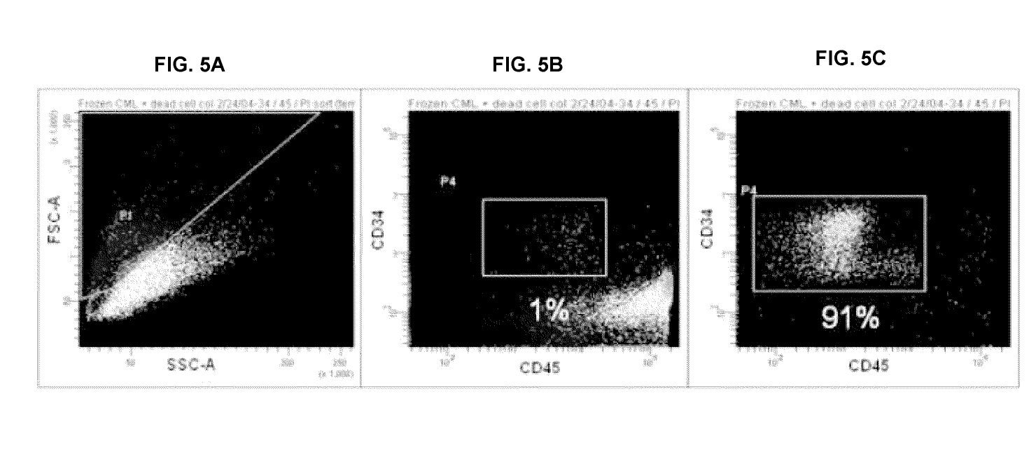

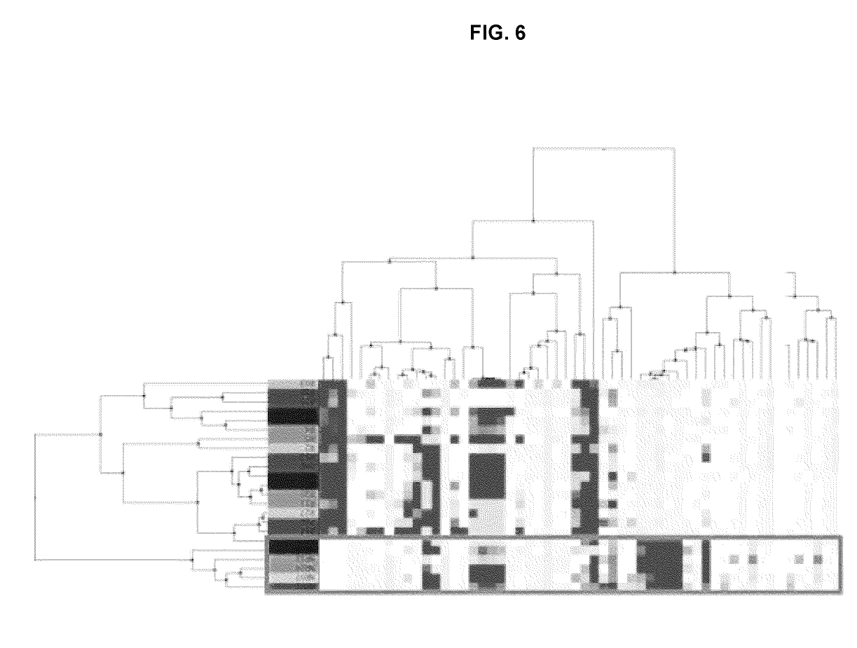

A BD FACSAria® (BD Biosciences) was used to sort CD34+ cells. Gates on forward scatter (FSC) and side scatter (SSC), followed by FSC-width (FSC-W) and FSC-height (FSC-H), were used to exclude dead cells and debris. Next, gates were set on PI negative cells to ensure that only viable cells were selected. Finally, on the CD34-FITC and CD45-PerCP-Cy5.5 histogram, CD45-PerCP-Cy5.5 dim cells that brightly coexpressed CD34-FITC were selected. The procedure was regarded as a success if greater than 1,000 CD34+ cells were isolated, with a purity of greater than 80% CD34+ cells by flow cytometry. An example of the sorting strategy is shown in FIG. 5. After sorting, CD34+ cells were placed in PicoPure® extraction buffer (Arcturus, Mountain View, Calif.) and stored at −80° C. until processed further. Small aliquots of CD34+ cells were also stored for fluorescence in-situ hybridization (FISH) to assess the proportion of BCR-ABL-positive cells. In the case of the validation set MNC were isolated from peripheral blood using density gradient centrifugation. CD34+ cells were isolated from the MNC using MiniMACS columns (Miltenyi Biotec, Bergisch-Gladbach, Germany), following the instructions of the manufacturer. An example of the sorting strategy is shown in Table 7.

TABLE 7
CD34+ cell isolation procedures summary

| Parameter | Value |
|---|---|
| Total number of BM mononuclear cells | |
| immediately post thaw | |
| Median | 1.4 × 10⁷ |
| Range | 1.5 × 10⁵-4.2 × 10⁷ |
| Viability of BM mononuclear cells | |
| immediately post thaw - % | |
| Median | 21.7 |
| Range | 1.4-86.2 |
| Number of viable BM mononuclear cells | |
| immediately prior to sorting | |
| Median | 1.9 × 10⁶ |
| Range | 7.2 × 10⁴-1.1 × 10⁷ |
| Viability of BM mononuclear cells | |
| immediately prior to sorting- % | |
| Median | 42.5 |
| Range | 6.2-91.7 |
| Purity of BM CD34+ cells immediately | |
| prior to sorting - % | |
| Median | 10.9 |
| Range | 1.7-68.6 |
| Number of CD34+ cells isolated and placed | |
| into RNA lysis buffer | |
| Median | 1.0 × 10⁴ |
| Range | 8.1 × 10¹-9.1 × 10⁴ |
| Purity of CD34+ cells isolated & placed | |
| into RNA lysis buffer - % | |
| Median | 95.9 |
| Range | 17.5-100 |

RNA Extraction and Gene Expression Profiling: RNA extraction for the training set was done in one batch on all 51 samples. The 23 samples of the validation set were processed as one batch in an identical fashion approximately 18 months after the training set. RNA extraction was performed with the PicoPure®RNA Isolation Kit (Arcturus) once all cell sorting had been completed. Samples were quantified using the NanoDrop® ND-1000 UV-Vis spectrophotometer (NanoDrop® Technologies, Wilmington, Del.) and the quality of the RNA was assessed using the Agilent 2100 Bioanalyzer (Agilent Technologies, Palo Alto, Calif.). Only samples with electropherograms showing a size distribution pattern predictive of acceptable microarray assay performance were processed further. To generate sufficient RNA for microarrray hybridization the GeneChip® Eukaryotic Small Sample Target Labeling Assay Version II (Affymetrix®, Santa Clara, Calif.) was used with adjustments for the lower than recommended input of starting RNA (5 to 10 ng instead of 20-100 ng). Following successful amplification, 5 μg of labelled target cRNA was hybridized to HG-U133 Plus 2.0 GeneChip arrays (Affymetrix®). Arrays were scanned using a laser confocal scanner (Agilent) and the image processing and expression analysis were performed using Affymetrix® GCOS v1.2 software. For QA/QC purposes, the parameters α1 and α2 were set to 0.05 and 0.065 (Affymetrix® defaults) respectively. These parameters set the point at which a probe set was called present (P), marginal (M) or absent (A). Minimal quality control parameters for inclusion in the study included P>30%, average signal in keeping with the average signal of other samples within that hybridization group (i.e. the group of samples hybridized as a batch), and a GAPDH 3′/5′ ratio of ≦3.62. Overall, the process of CD34+ cells selection, RNA extraction and array hybridization was successful in 36 of 51 patients (71%). The average present call rate in this group was of 41.5% (range, 38.8% to 47.1%). FISH for BCR-ABL was successful in 28 out of the 36 samples. The median percentage of BCR-ABL positive CD34+ cells was found to be 98.5% (33-100%). The 23 samples of the validation set were processed in an identical fashion approximately 18 months after the training set. For consistency, similar amounts of input RNA (2-20 ng) were used.

Patient demographics: Differences in the distribution of patient demographics/treatment history were examined by categorical data analysis in the training set using the SPSS software package.

Statistical Analysis: Standard analysis tools were applied to patient characteristics. Low-level analysis of the Affymetrix data was conducted using the Robust Multi-array Average (RMA) algorithm (Irizarry et al., Biostatistics 4(2):249-64, 2003). Transcript-by-transcript ANOVA to determine differential expression between non-responders and responders was performed on the training set. Testing of the classifier was performed on the independent, blinded validation set. With regard to downstream analysis of the classifier, overrepresented gene ontology and pathway annotations were identified in the classifier transcripts using categorical data analysis. Known protein-protein interactions were examined for classifier members as well as with other genes using the Metacore Database™.

### Microarray Data Analysis:

Low Level Analysis: Low-level analysis of the Affymetrix data was conducted using the Robust Multi-array Average (RMA) algorithm (Irizarry et al., Biostatistics 4(2):249-64, 2003). Only Perfect Match intensities were used. Parameters for RMA included model-based background correction, quantile normalization and median polish. Transcript-by-transcript (i.e., unique Affymetrix Probe set IDs)

Feature Selection: ANOVA to determine differential expression between NR and R was performed on the training set (N=36). All p-values were False Discovery Rate (FDR) adjusted. With respect to feature selection was based on effect size (fold change (FC)>|1.5|) and statistical significance (p-value <0.1) to minimize false negatives. Data was further filtered based on threshold expression level and variability (based on CV).

Class prediction was performed using the nearest shrunken centroids algorithm (Tibshirani, Hastie, Narasimhan, and Chu, 2002). Parameters for the classification algorithms were chosen by nested cross-validation procedures to optimize performance while avoiding overfitting. Testing of the classifier was performed on an independent, blinded validation set (N=23). Finally, resampling was performed on the classifier list to determine the minimal subset (N=75).

Structural analysis of the classifier: With regard to downstream analysis of the classifier, over-represented gene ontology and pathway annotations were identified in the classifier transcripts using categorical data analysis (with adjustment for the nested multiple comparisons). Known protein/protein interactions were examined for classifier members as well as with other genes using the Metacore Database™. In addition to examining functional enrichment, potential sub-networks (or “small networks”) in the classifier were examined using known and curated protein-protein interactions from the MetaCore Database™. These subnetworks were ranked based on statistical significance and the number of known biological pathways found in the sub-network. The p-values are based on a hypergeometric distribution in which the p-value essentially represents the probability of particular mapping arising by chance, given the numbers of genes in the set of all genes on maps/networks/processes, genes on a particular map/network/process, and genes in the experiment. This is formally defined as:

$$p \text{-} Value = \frac{R ! n ! ( N - R ) ! ( N - n ) !}{N !} {\sum }_{i = max ( r , R + n - N )}^{min ( n , R )} \frac{1}{i ! ( R - i ) ! ( n - i ) ! ( N - R - n + i ) !}$$

where N=total number of nodes in MetaCore Database™; R=number of the network's objects corresponding to the genes and proteins in your list; n=total number of nodes in each small network generated from your list; r=number of nodes with data in each small network generated (O'Brien et al., N Engl J Med 348(11):994-1004, 2003).

Meta-analysis: CEL files for the Yong et al paper were provided by the authors. The data was analyzed similarly to that of the training set (RMA normalization, one-way ANOVA). Reported fold changes and p-values for the Zheng et al data set were downloaded from the journal website. Overlap was calculated based on the number of shared putative differentially expressed genes. Simulations in the statistical computing environment R were performed to determine the number of overlapping features (0) expected to be shared among two candidate lists of different lengths (n1, n2) both sampled from the same array (with N features). Statistical significance was determined by comparing the observed value (o) with the distribution generated from 10,000 simulations performed for a given configuration (n1, n2, N).

Downstream Analyses: Statistically over-represented high frequency transcripts from the classifiers were examined for both Gene Ontology and Pathway Annotation. As part of the process of Gene Ontology over-representation analysis, transcripts are grouped by functional relationships. Overlaying expression (i.e., up or down regulation) allows for the identification of functional groups that have similar patterns. Finally, the 2 kb upstream region of the transcripts in the classifier was examined for over-represented or shared motifs based on data from TRANSFAC®.

Baseline characteristics of the training set: Overall, the process of CD34+ cell selection, RNA extraction and array hybridization was successful in 36 of 51 patients (71%), amongst them 24 non-responders and 12 responders. FISH for BCR-ABL was successful in 28 of 36 patients (78%) and revealed between 33 and 100% (median 98.5%) BCR-ABL-positive interphases, with a small but statistically significant difference between non-responders and responders (median of 100% vs. 98.5%, P=0.01). Compared to responders, nonresponders tended to be older (P=0.048) and had a longer interval between diagnosis and imatinib start (P=0.037) (Table 1).

TABLE 1
Clinical characteristics of the training set

| Characteristic | | | P Value |
|---|---|---|---|
| Male sex - no. (%) | | | |
| Responders | 15 | (63) | 1.00 |
| Non-responders | 7 | (58) | |
| Age (at diagnosis)- years (median, range) | | | |
| Responders | 51 | (28-76) | 0.048 |
| Non-responders | 61 | (24-71) | |
| Hemoglobin - g/dl | | | |
| Responders | 13.1 | (10.0-16.3) | 0.575 |
| Non-responders | 12.5 | (10.3-15.8) | |
| White cell count - ×10³/l | | | |
| Responders | 12.0 | (2.5-70.8) | 0.373 |
| Non-responders | 17.8 | (4.7-116) | |
| Platelet count - ×10³/l | | | |
| Responders | 265.5 | (19-935) | 0.098 |
| Non-responders | 350 | (99-1372) | |
| Peripheral blood basophil count - % | | | |
| Responders | 4 | (0-31) | 0.938 |
| Non-responders | 6 | (0-16) | |
| Peripheral blood eosinophil count - % | | | |
| Responders | 1 | (0-8) | 0.441 |
| Non-responders | 2 | (0-3) | |
| Peripheral blood blast count - % | | | |
| Responders | 0 | (0-11) | 0.657 |
| Non-responders | 0 | (0-5) | |
| Bone marrow blast count - % | | | |
| Responders | 1 | (0-13) | 0.234 |
| Non-responders | 3 | (0-18) | |
| Spleen size (cm below costal margin) | | | |
| Responders | 0 | (0-11) | 0.806 |
| Non-responders | 0 | (0-10) | |
| Chronic phase with CE* | | | |
| Responders | 6 | (26) | 0.874 |
| Non-responders | 3 | (27) | |
| Deletion of deriv. chromosome 9 - no. (%) | | | |
| Responders | 2 | (8) | 0.717 |
| Non-responders | 1 | (8) | |
| Prior hydroxyurea therapy | | | |
| Responders | 20 | (83) | 0.180 |
| Non-responders | 12 | (100) | |
| Prior interferon-α therapy | | | |
| Responders | 19 | (79) | 0.113 |
| Non-responders | 12 | (100) | |
| Other prior therapy | | | |
| Responders | 7 | (29) | 0.092 |
| Non-responders | 7 | (58) | |
| Initial imatinib dose 600 mg | | | |
| daily - no. (%)** | | | |
| Responders | 10 | (43) | 0.255 |
| Nonresponders | 3 | (25) | |
| Time from diagnosis to imatinib | | | |
| therapy - days | | | |

| | | |
|---|---|---|
| Responders | 928 | 0.037 |
| Non-responders | 1812 | |

CE—clonal cytogenetic evolution.
*Two patients (1 responder and 1 non-responder) were subsequently found to fulfill the criteria for accelerated phase (platelet count <100/nL unrelated to therapy, and basophils in the blood >20%).
**Patients with CE were classified as in accelerated phase in the phase 2 imatinib studies (but not in the IRIS study) and therefore treated with an initial dose of 600 mg imatinib daily

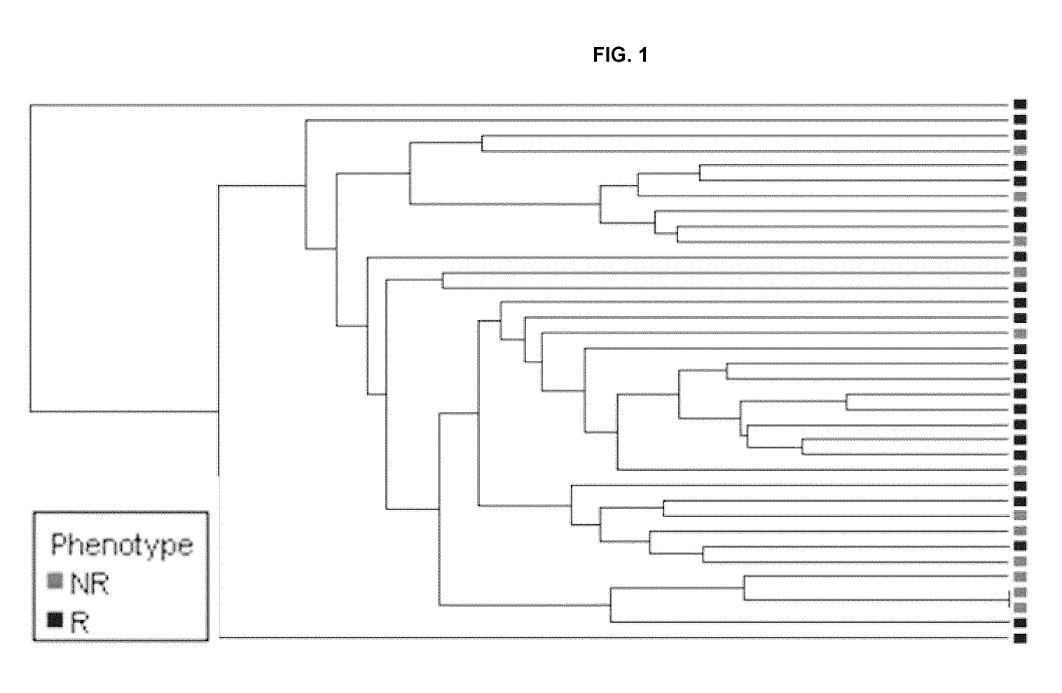

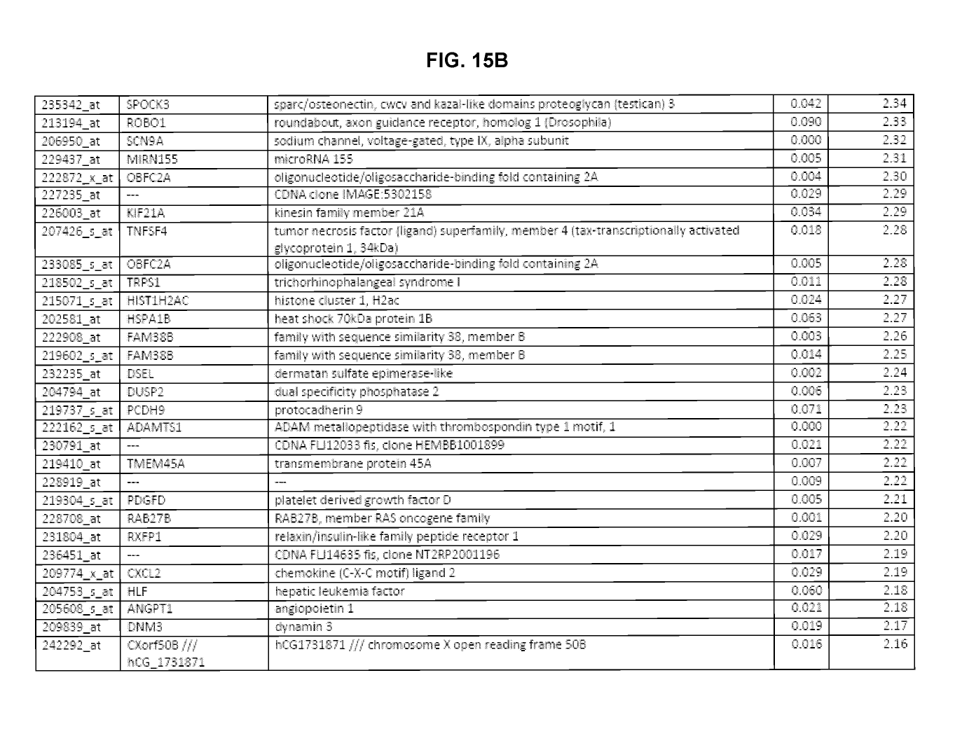

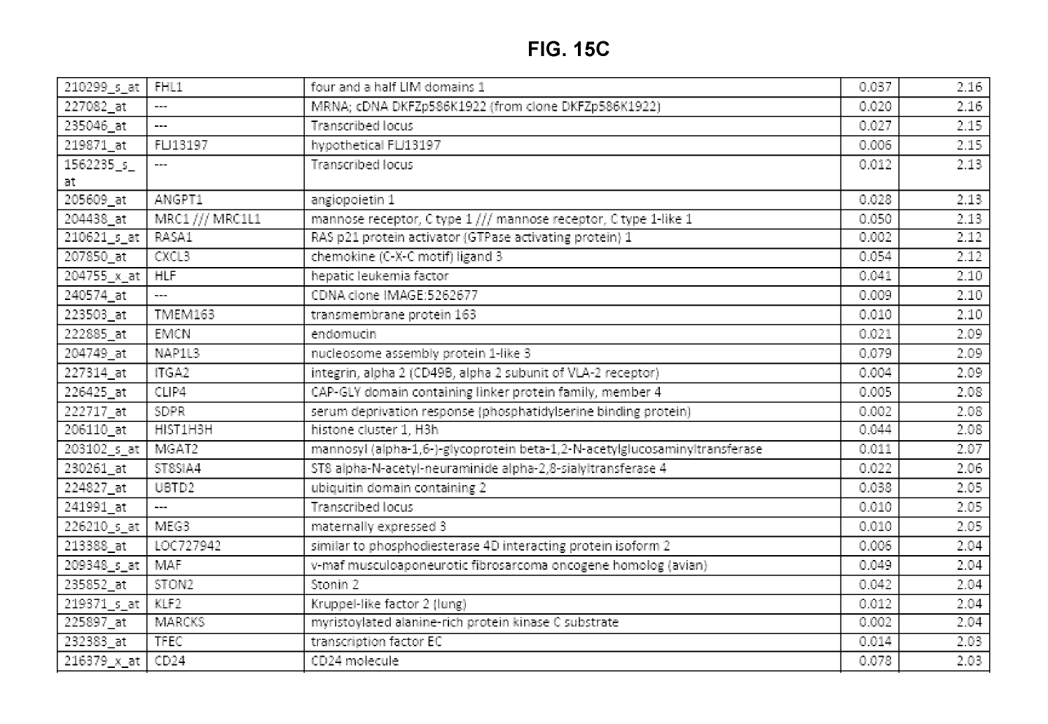

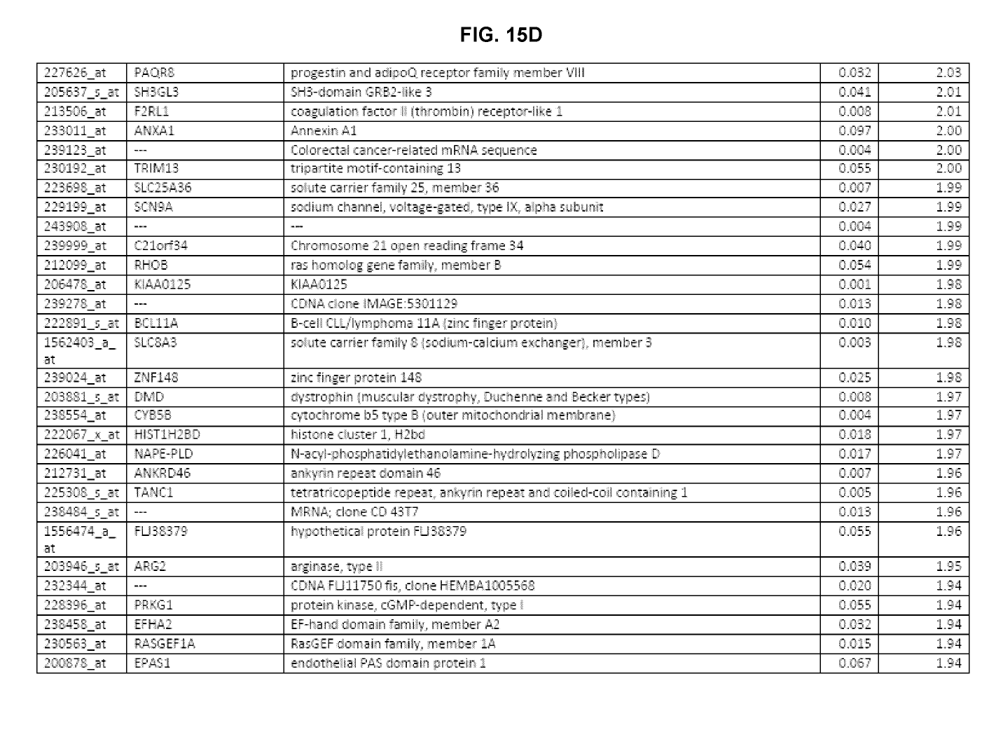

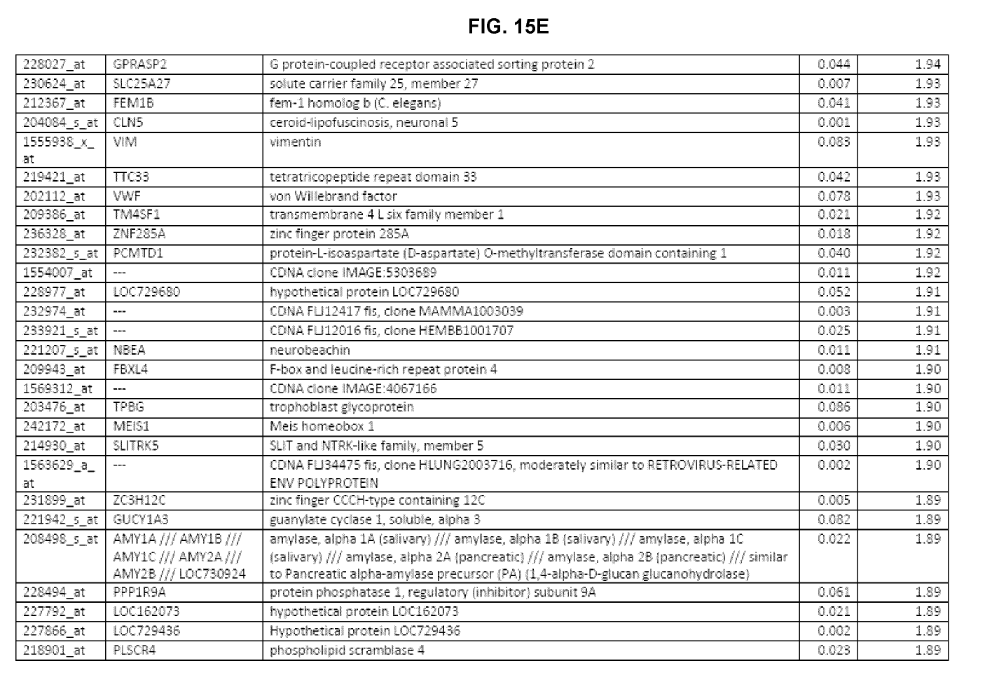

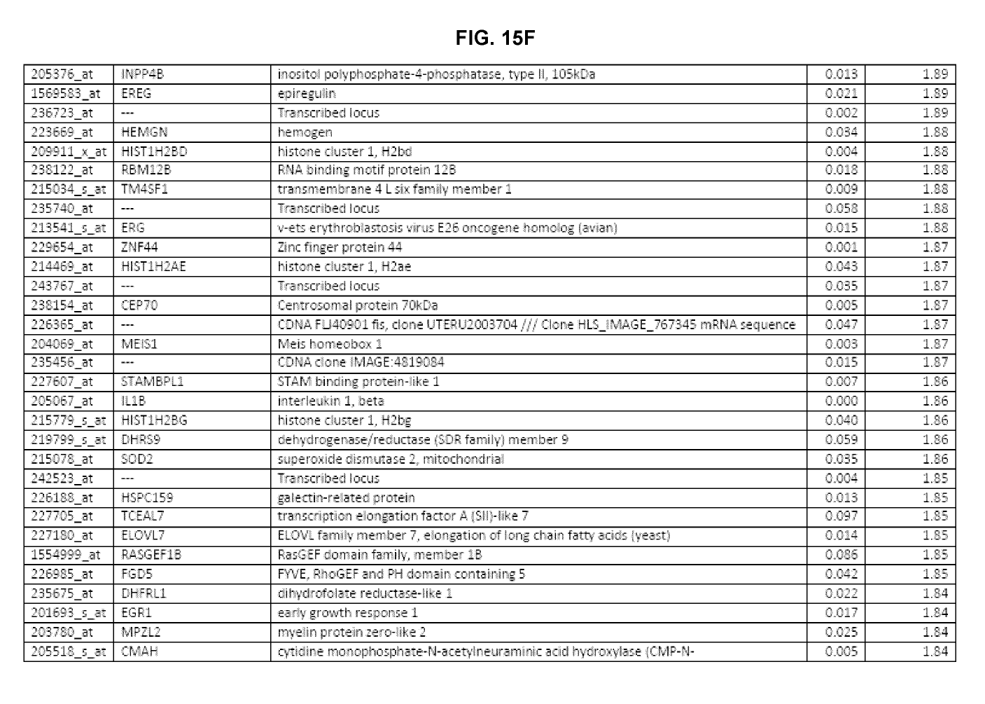

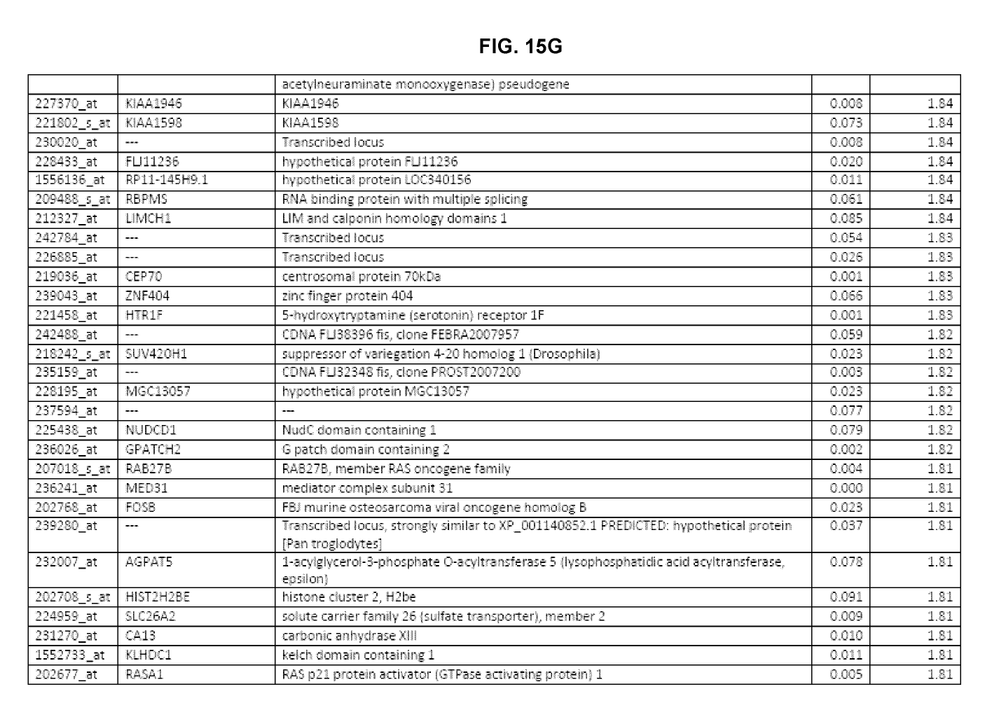

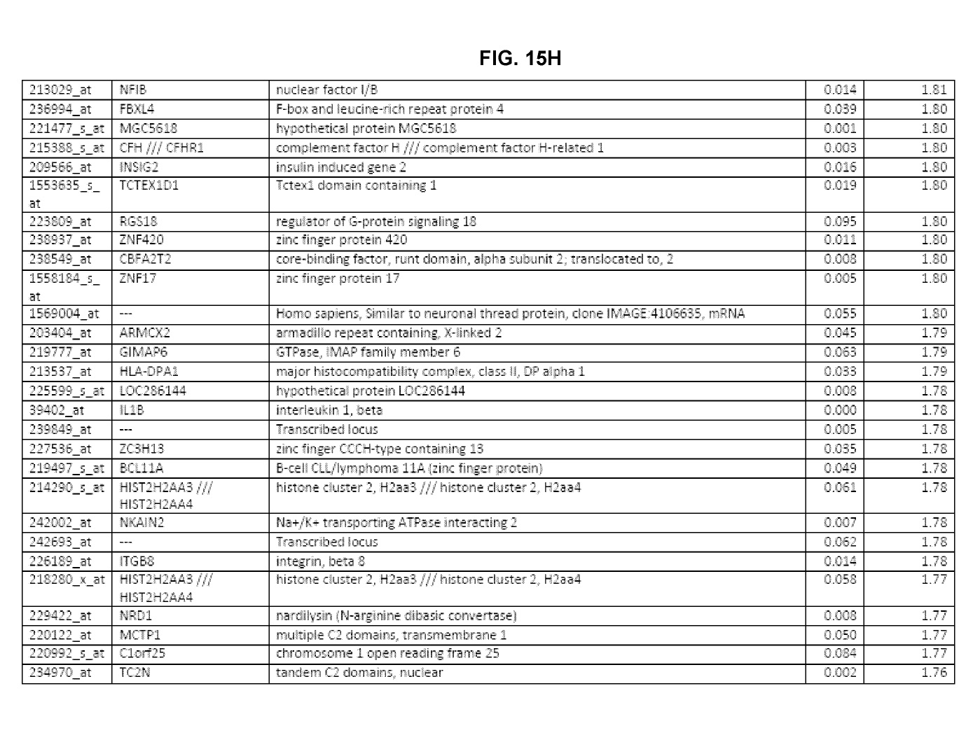

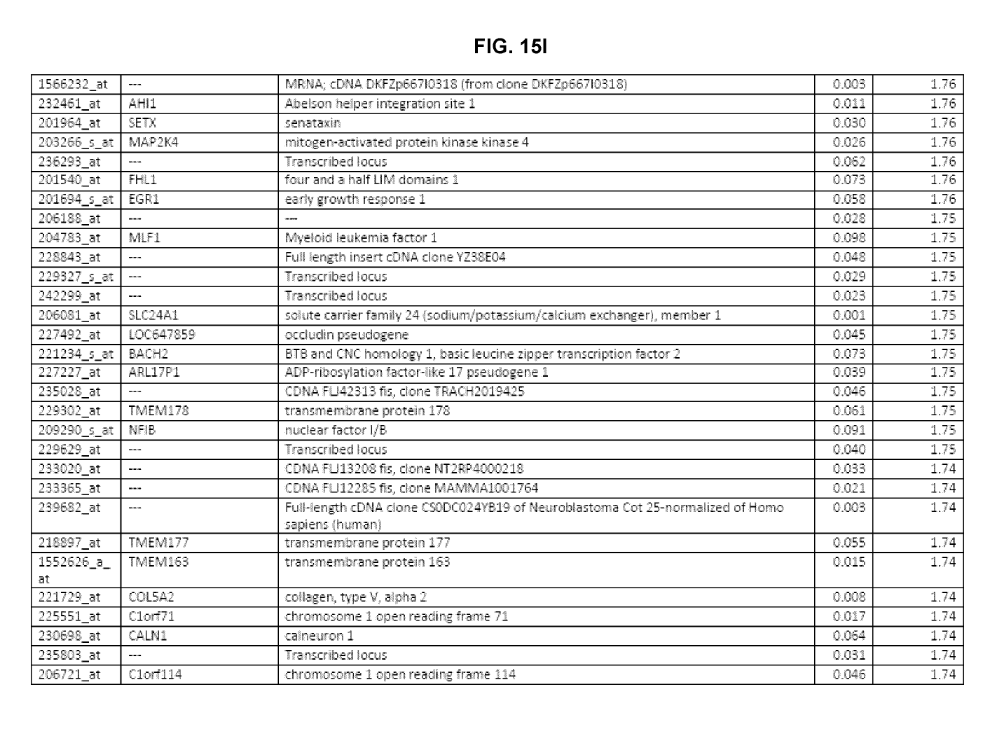

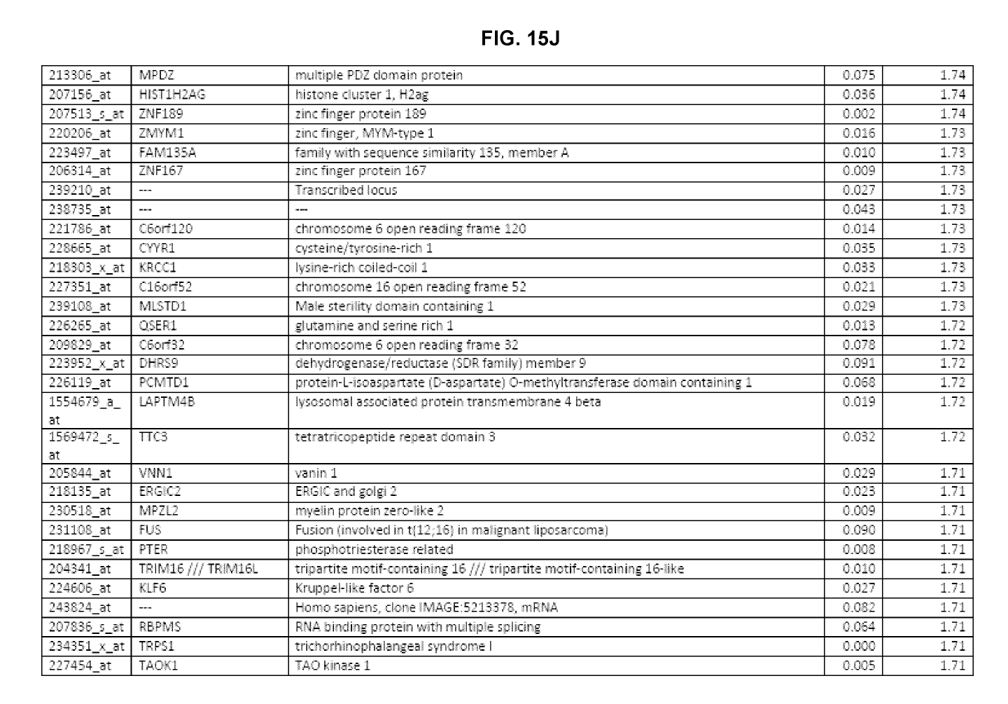

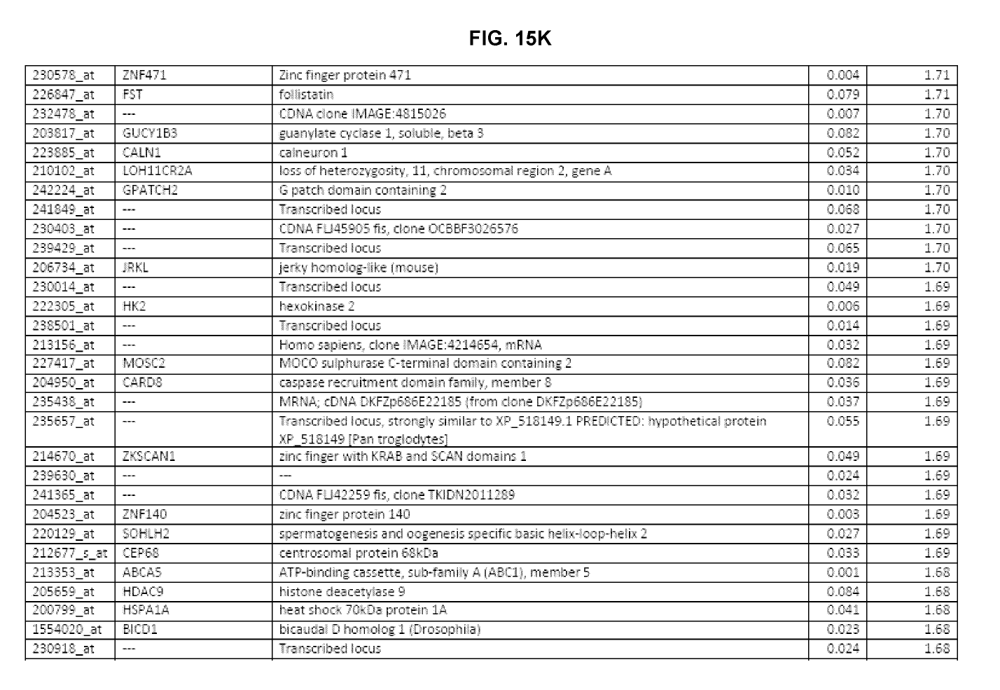

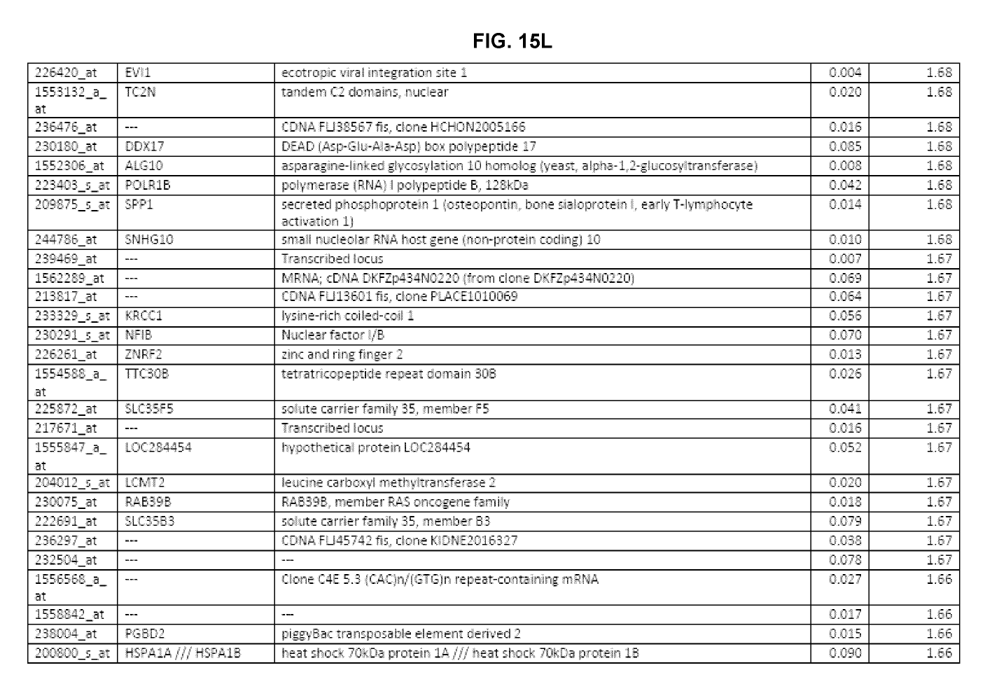

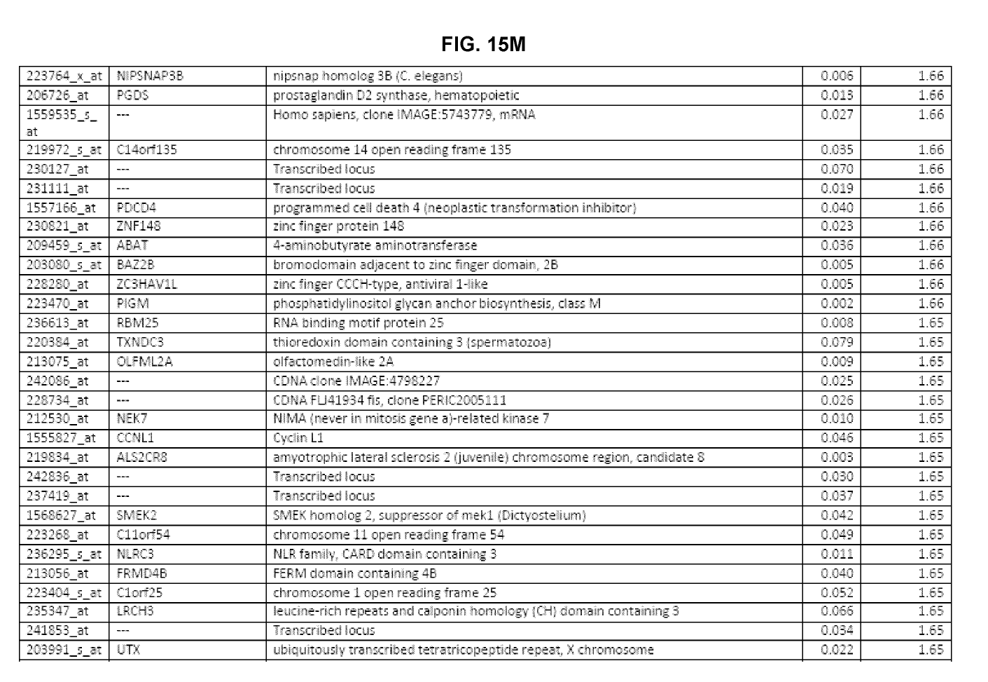

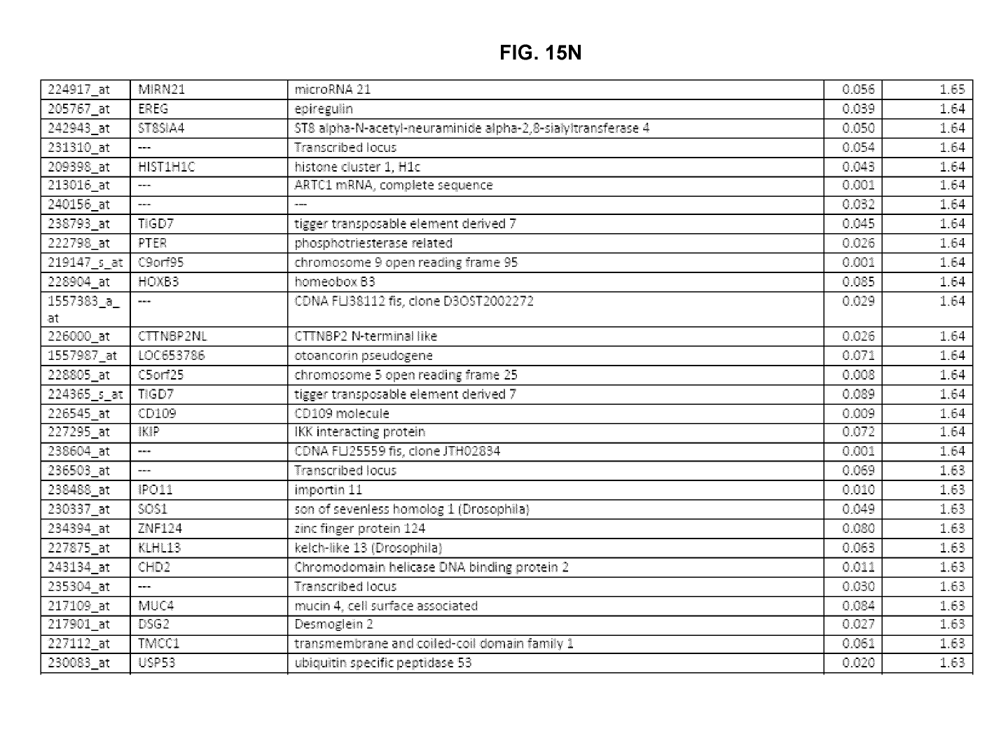

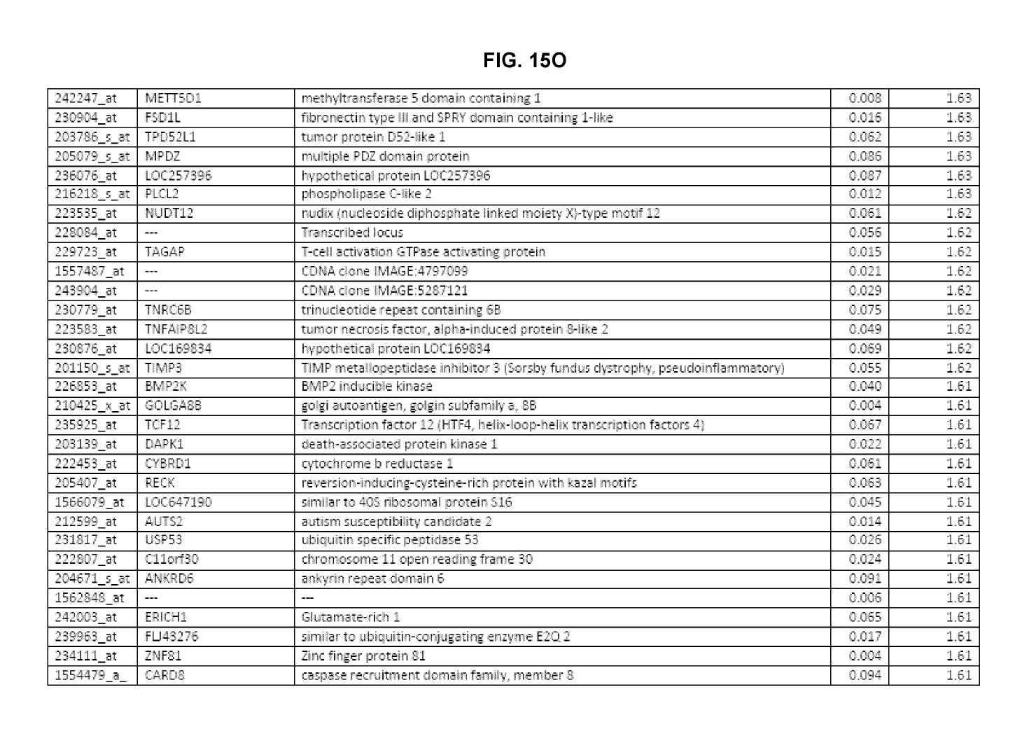

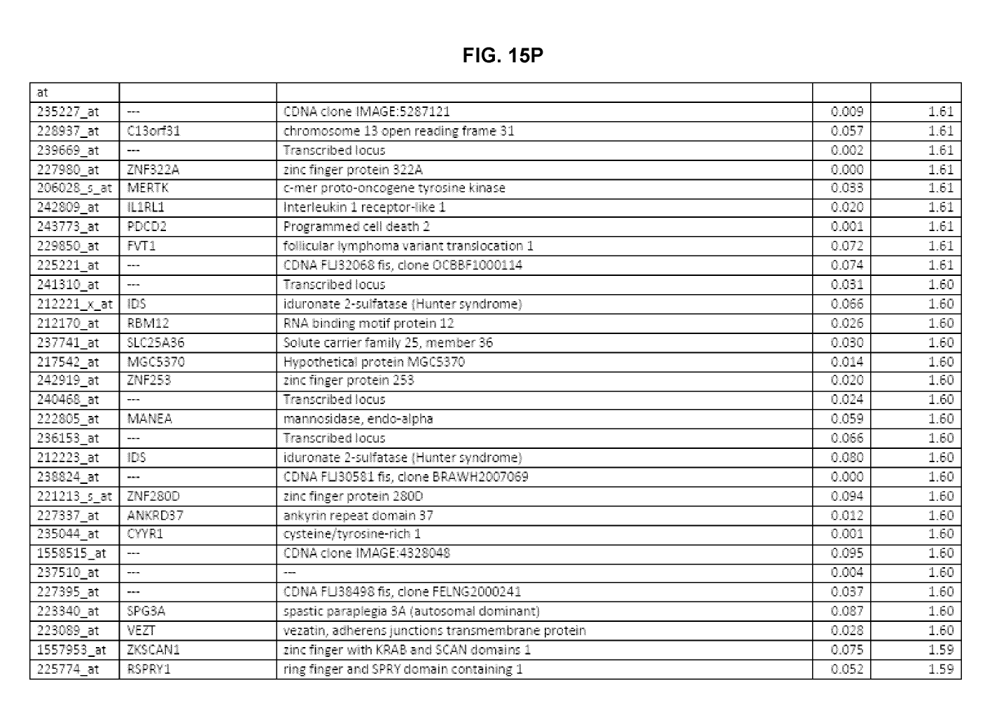

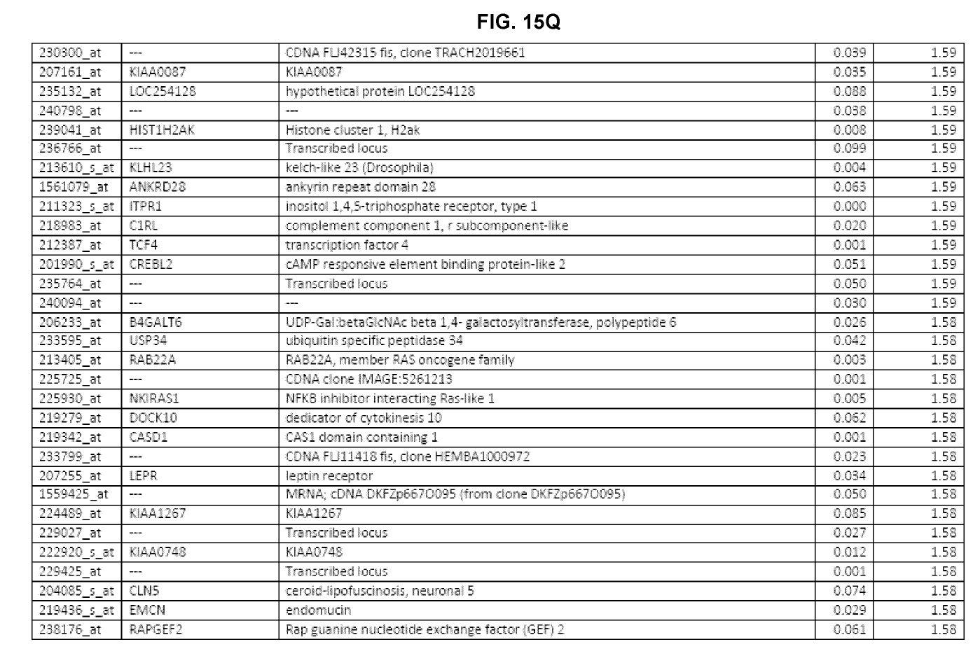

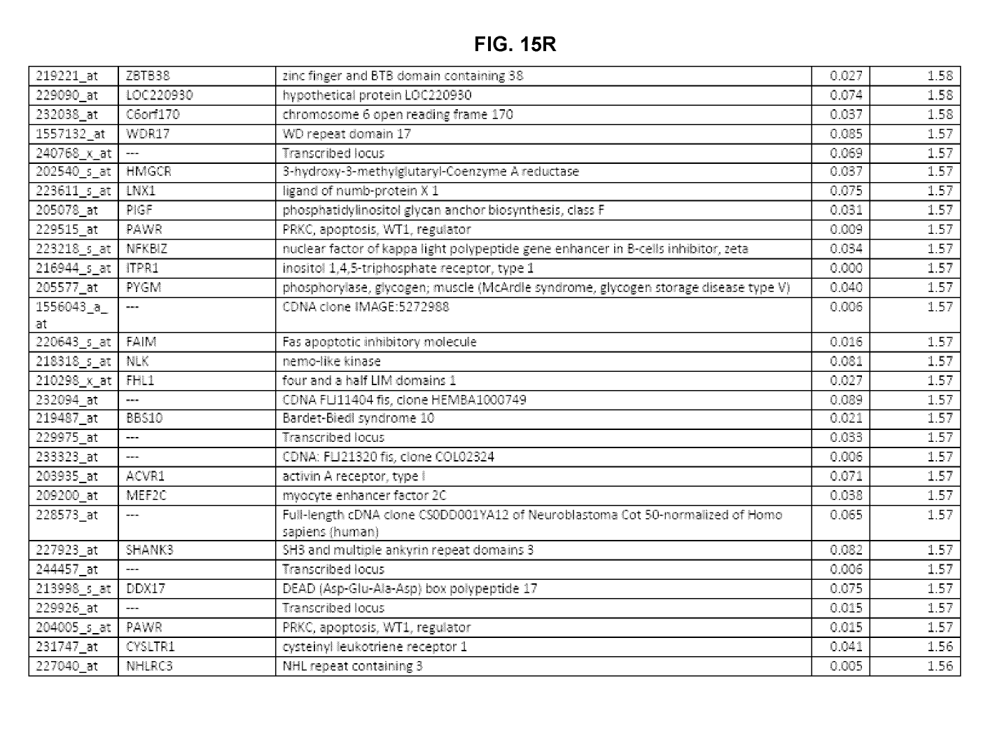

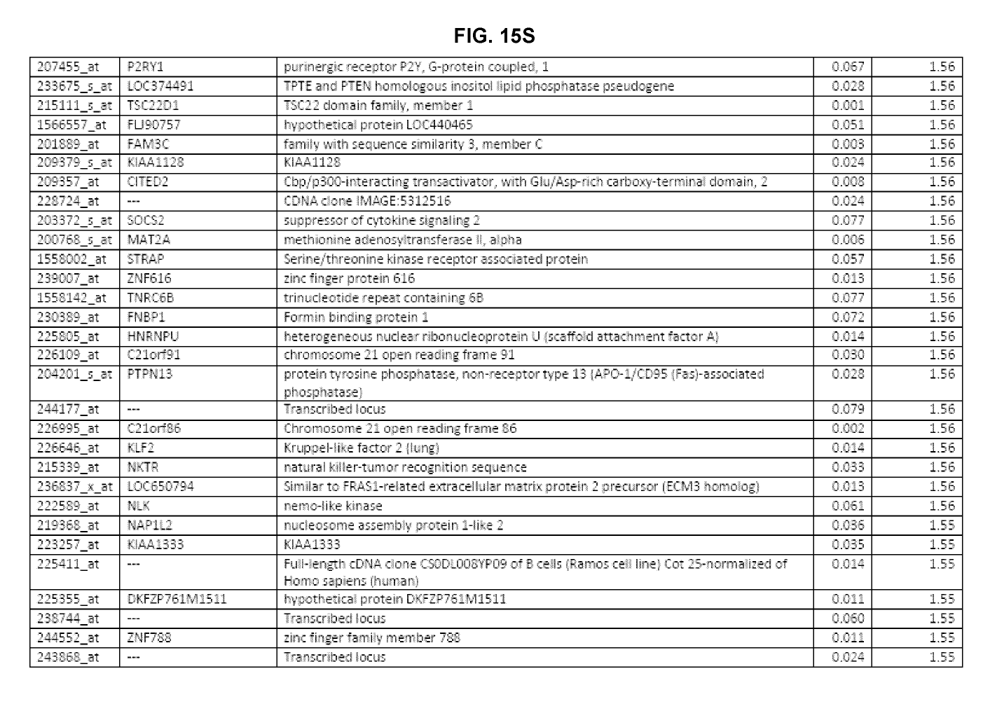

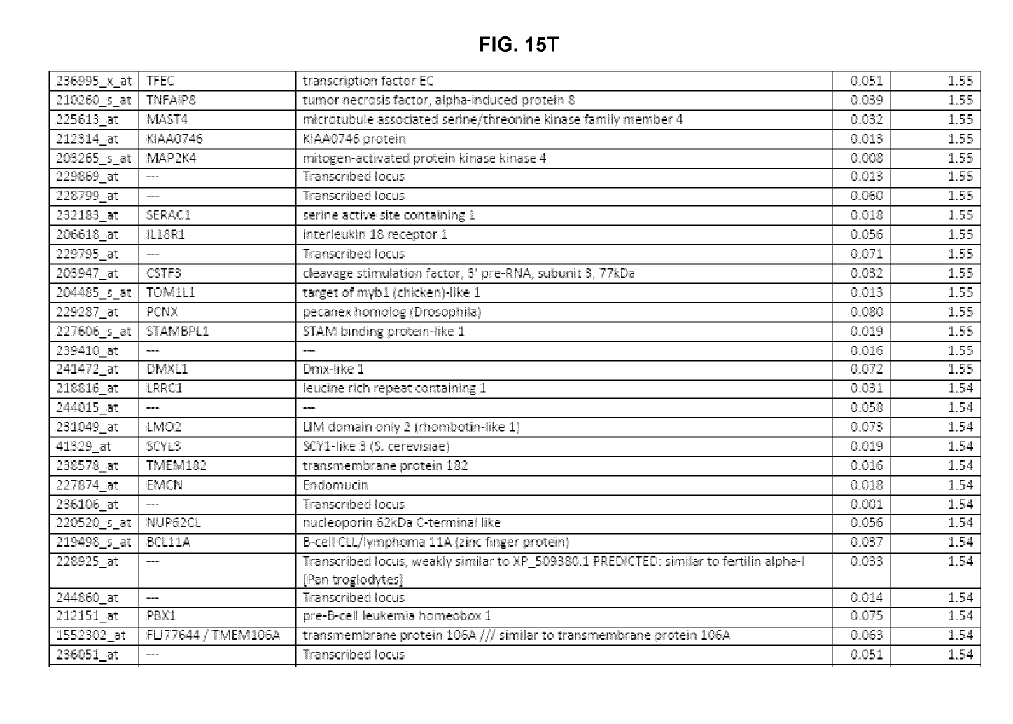

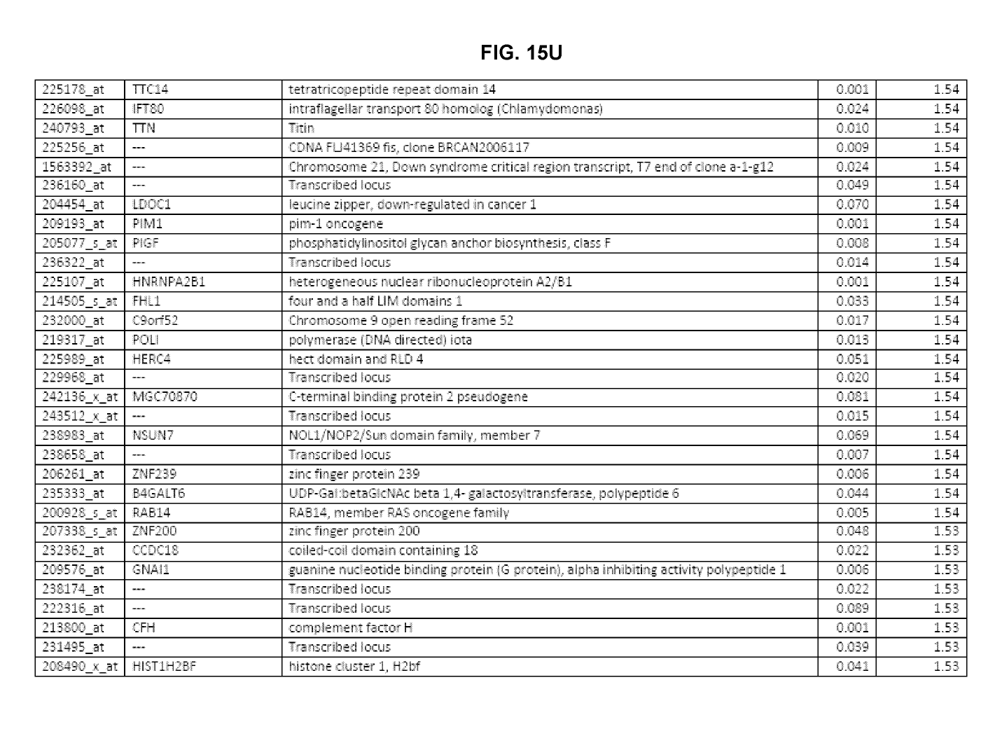

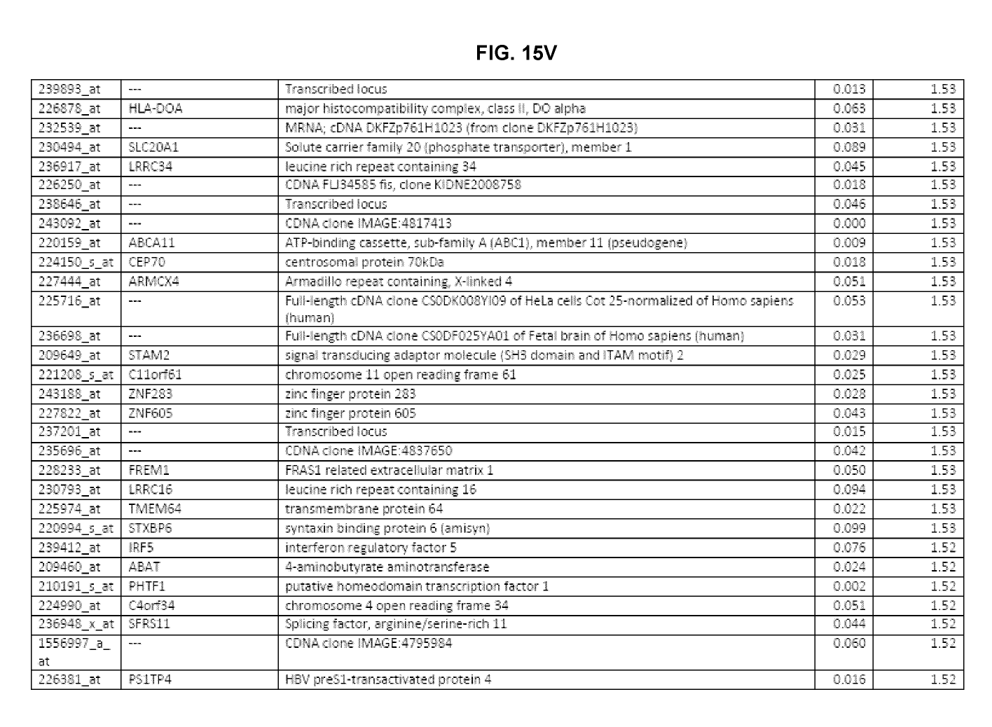

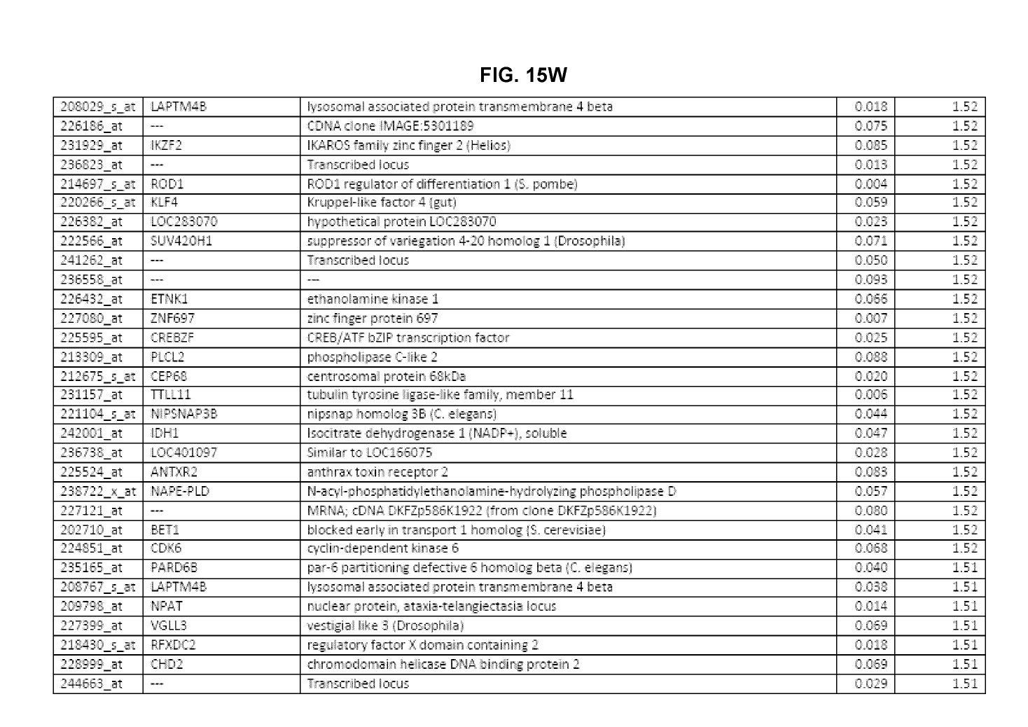

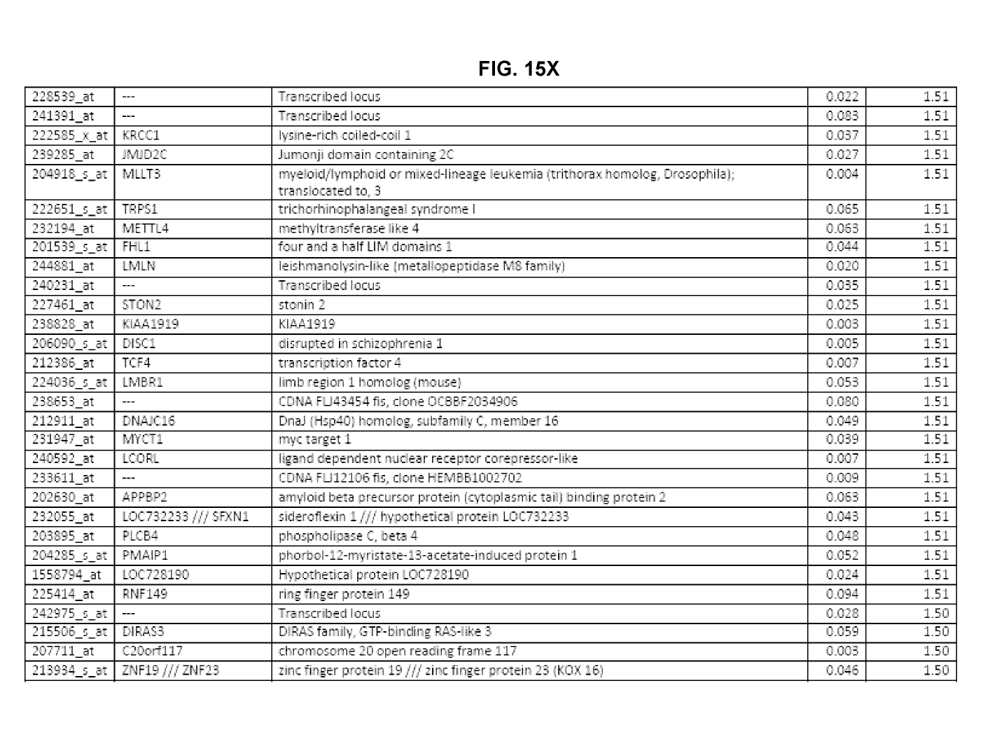

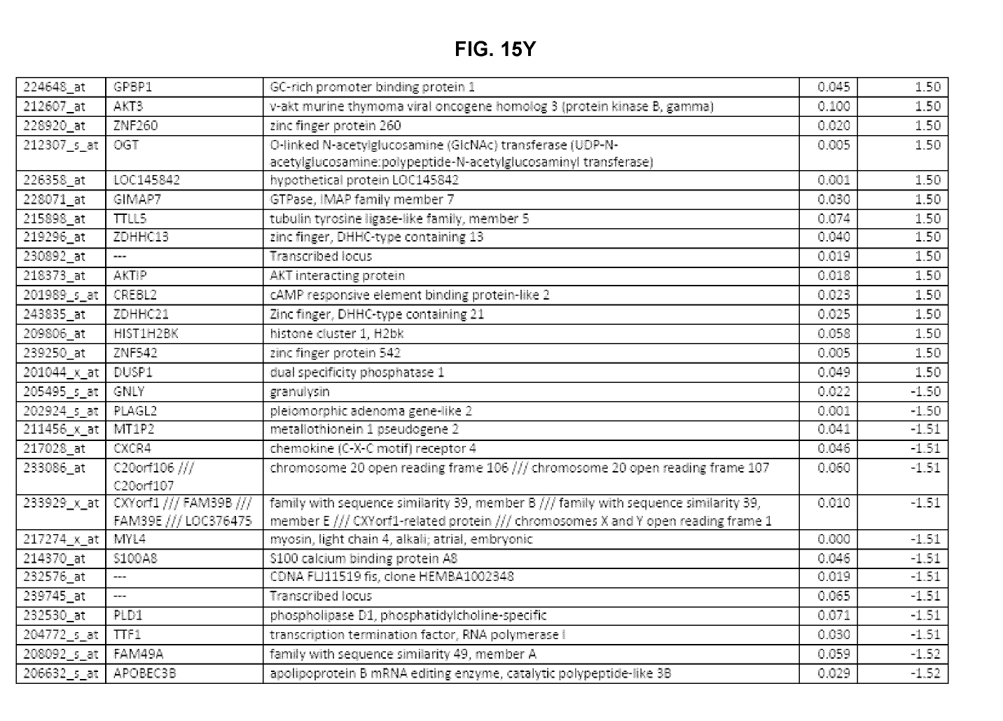

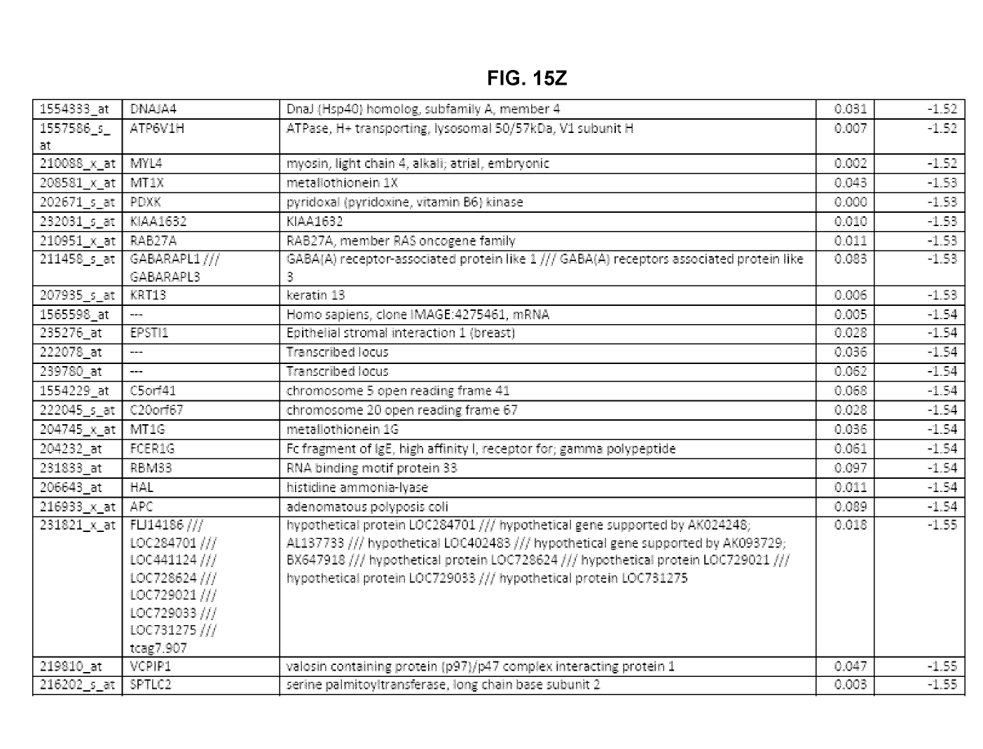

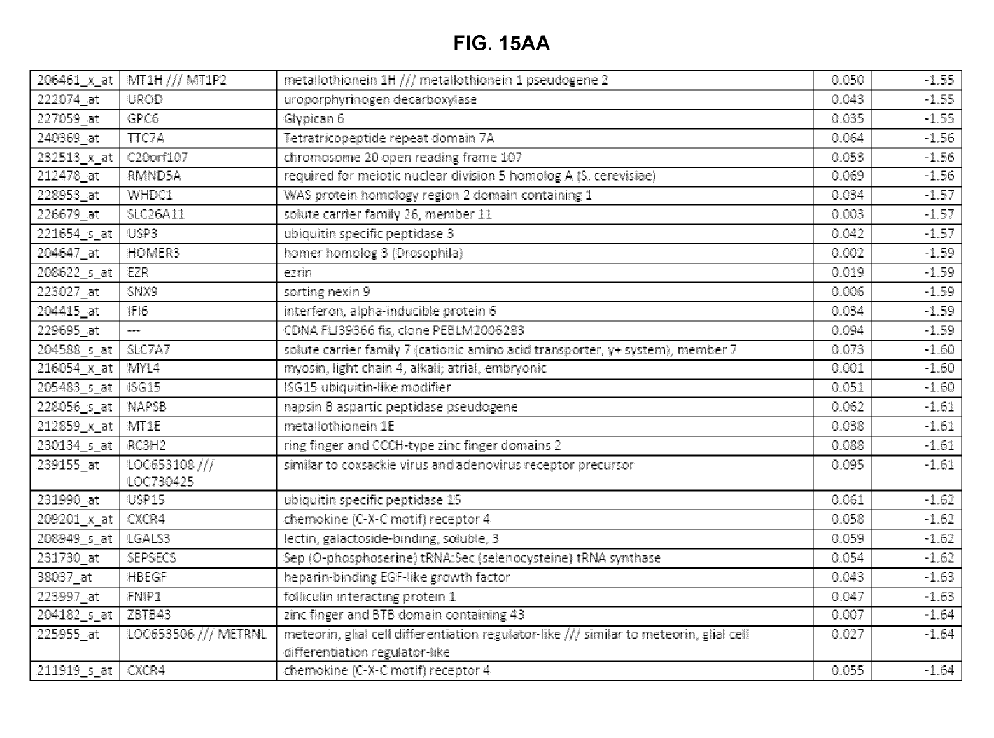

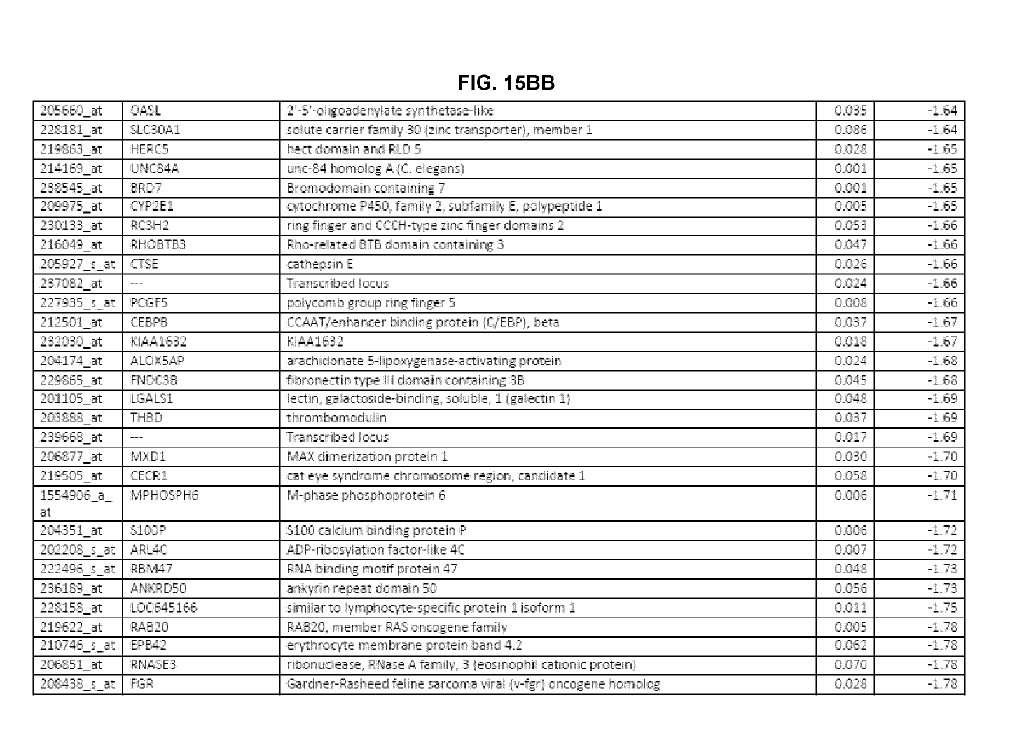

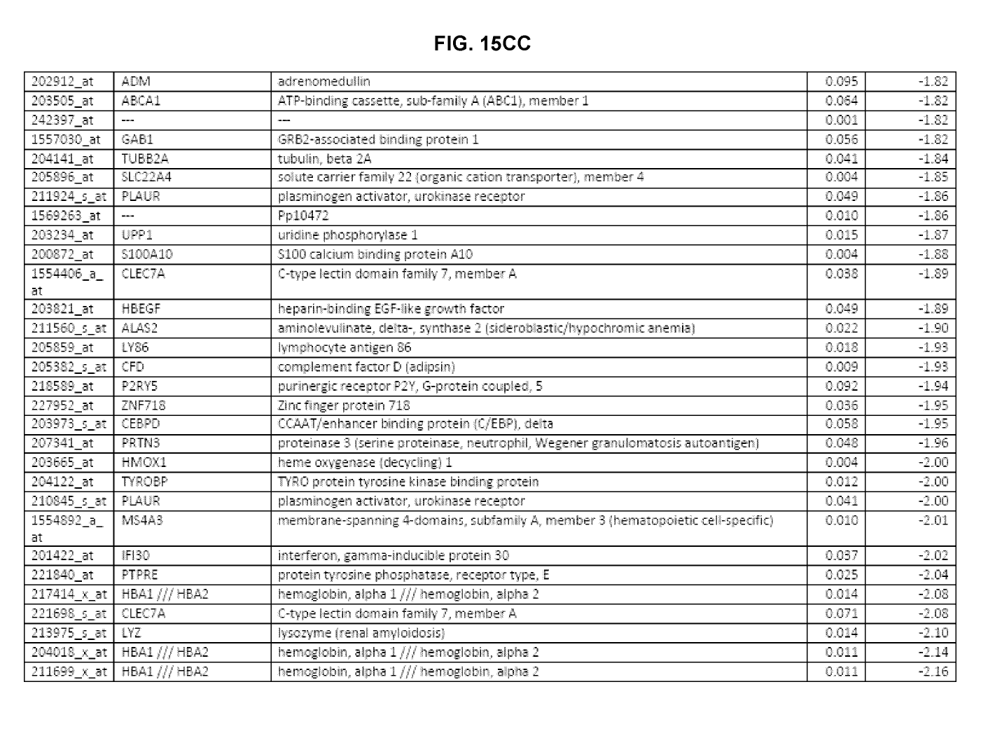

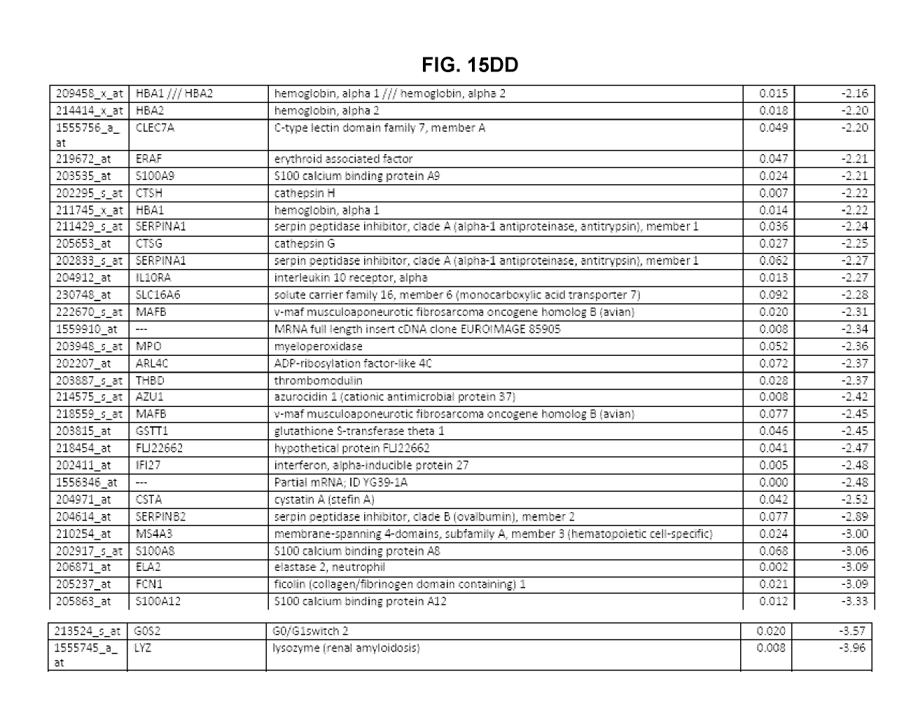

Construction of the response classifier: To determine whether the gene expression profiles of CD34+ cells from prospective cytogenetic responders and non-responders were different, unsupervised hierarchical cluster analysis was performed. Partial separation between responders and non-responders FIG. 1. Univariate analysis of the training set identified 885 differentially expressed transcripts based on minimal effect size [fold change (FC)>|1.5| and p-value (<0.1)] (see FIG. 15A-DD, Table 6). The prediction analysis for microarrays (PAM) algorithm was then applied to the training set and classification accuracy was determined via cross validation. Cross-validation was used to choose an optimum gene number (threshold), which minimized classification errors and resulted in a 75 transcript predictor (Table 2). Fifty of these transcripts were up-regulated and twenty-five were down-regulated in non-responders vs. responders.

TABLE 2
Probe sets (transcripts) of the minimal response classifier

| | | Training | | Test set | β-Catenin |
|---|---|---|---|---|---|
| | Gene | set fold | Training | fold | target |
| Probeset | Symbol | change | p-value | change | by SACO |
| 225688_s_at | PHLDB2 | 4.197 | 0.009 | 1.646 | Yes |
| 205848_at | GAS2 | 3.400 | 0.021 | 2.115 | No |
| 219454_at | EGFL6 | 3.302 | 0.010 | 1.853 | No |
| 238206_at | RXFP1 | 2.829 | 0.011 | 2.290 | No |
| 205612_at | MMRN1 | 2.412 | 0.012 | 1.862 | Yes |
| 229963_at | NGFRAP1L1 | 2.410 | 0.038 | 1.802 | No |
| 235342_at | SPOCK3 | 2.337 | 0.042 | 2.515 | Yes |
| 226003_at | KIF21A | 2.287 | 0.034 | 1.672 | No |
| 230791_at | FLJ12033 | 2.224 | 0.021 | 1.551 | No |
| 205609_at | ANGPT1 | 2.129 | 0.028 | 1.732 | No |
| 223503_at | TMEM163 | 2.098 | 0.010 | 1.594 | Yes |
| 222885_at | EMCN | 2.095 | 0.021 | 1.765 | Yes |
| 227314_at | ITGA2 | 2.086 | 0.004 | 1.489 | Yes |
| 226425_at | CLIP4 | 2.084 | 0.005 | 1.474 | Yes |
| 205637_s_at | SH3GL3 | 2.013 | 0.041 | 1.972 | Yes |
| 1562403_a_at | SLC8A3 | 1.979 | 0.003 | 1.725 | Yes |
| 228396_at | PRKG1 | 1.940 | 0.055 | 2.240 | No |
| 228027_at | GPRASP2 | 1.938 | 0.044 | 1.664 | No |
| 202112_at | VWF | 1.927 | 0.078 | 3.179 | Yes |
| 1554007_at | BC041986 | 1.918 | 0.011 | 1.562 | No |
| 223669_at | HEMGN | 1.881 | 0.034 | 1.483 | Yes |
| 229654_at | ZNF44 | 1.875 | 0.001 | 1.458 | Yes |
| 204069_at | MEIS1 | 1.871 | 0.003 | 1.360 | Yes |
| 205518_s_at | CMAH | 1.842 | 0.005 | 1.553 | No |
| 221802_s_at | KIAA1598 | 1.840 | 0.073 | 2.099 | Yes |
| 1556136_at | RP11-145H9.1 | 1.837 | 0.011 | 1.607 | Yes |
| 209488_s_at | RBPMS | 1.836 | 0.061 | 1.855 | Yes |
| 228195_at | MGC13057 | 1.820 | 0.023 | 1.702 | Yes |
| 213029_at | NFIB | 1.806 | 0.014 | 1.865 | Yes |
| 203404_at | ARMCX2 | 1.792 | 0.045 | 1.467 | No |
| 226189_at | ITGB8 | 1.779 | 0.014 | 1.390 | Yes |
| 209290_s_at | NFIB | 1.746 | 0.091 | 2.390 | Yes |
| 1552626_a_at | TMEM163 | 1.742 | 0.015 | 1.442 | Yes |
| 230698_at | CALN1 | 1.741 | 0.064 | 1.678 | No |
| 213306_at | MPDZ | 1.737 | 0.075 | 1.704 | No |
| 230518_at | EVA1 | 1.711 | 0.009 | 1.478 | No |
| 207836_s_at | RBPMS | 1.708 | 0.064 | 1.507 | Yes |
| 210102_at | LOH11CR2A | 1.702 | 0.034 | 1.487 | Yes |
| 227417_at | MOSC2 | 1.691 | 0.082 | 1.519 | Yes |
| 204523_at | ZNF140 | 1.688 | 0.003 | 1.543 | No |
| 230291_s_at | NFIB | 1.672 | 0.070 | 1.994 | Yes |
| 209459_s_at | ABAT | 1.657 | 0.036 | 1.504 | Yes |
| 228805_at | C5orf25 | 1.637 | 0.008 | 1.564 | No |
| 227875_at | KLHL13 | 1.632 | 0.063 | 1.594 | Yes |
| 217109_at | MUC4 | 1.630 | 0.084 | 1.482 | Yes |
| 203786_s_at | TPD52L1 | 1.627 | 0.062 | 1.954 | Yes |
| 205079_s_at | MPDZ | 1.627 | 0.086 | 1.367 | No |
| 201150_s_at | TIMP3 | 1.616 | 0.055 | 1.826 | Yes |
| 235227_at | BC043173 | 1.609 | 0.009 | 1.736 | No |
| 242919_at | ZNF253 | 1.602 | 0.020 | 1.476 | No |
| 212501_at | CEBPB | 0.598 | 0.037 | 0.459 | No |
| 219505_at | CECR1 | 0.587 | 0.058 | 0.425 | Yes |
| 202208_s_at | ARL4C | 0.580 | 0.007 | 0.554 | No |
| 222496_s_at | FLJ20273 | 0.579 | 0.048 | 0.516 | Yes |
| 202912_at | ADM | 0.549 | 0.095 | 0.381 | Yes |
| 242397_at | AI694722 | 0.549 | 0.001 | 0.658 | No |
| 205896_at | SLC22A4 | 0.541 | 0.004 | 0.579 | Yes |
| 1569263_at | AF318321 | 0.537 | 0.010 | 0.445 | No |
| 203234_at | UPP1 | 0.535 | 0.015 | 0.478 | Yes |
| 200872_at | S100A10 | 0.531 | 0.004 | 0.611 | Yes |
| 218589_at | P2RY5 | 0.515 | 0.092 | 0.532 | No |
| 201422_at | IFI30 | 0.494 | 0.037 | 0.440 | No |
| 221840_at | PTPRE | 0.491 | 0.025 | 0.386 | Yes |
| 221698_s_at | CLEC7A | 0.480 | 0.071 | 0.434 | No |
| 211429_s_at | SERPINA1 | 0.446 | 0.036 | 0.335 | Yes |
| 205653_at | CTSG | 0.445 | 0.027 | 0.421 | No |
| 202833_s_at | SERPINA1 | 0.441 | 0.062 | 0.270 | Yes |
| 230748_at | SLC16A6 | 0.439 | 0.092 | 0.514 | Yes |
| 222670_s_at | MAFB | 0.432 | 0.020 | 0.567 | No |
| 203948_s_at | MPO | 0.423 | 0.052 | 0.551 | Yes |
| 202207_at | ARL4C | 0.423 | 0.072 | 0.319 | No |
| 218454_at | FLJ22662 | 0.405 | 0.041 | 0.324 | No |
| 204971_at | CSTA | 0.397 | 0.042 | 0.464 | No |
| 210254_at | MS4A3 | 0.334 | 0.024 | 0.376 | No |
| 205237_at | FCN1 | 0.324 | 0.021 | 0.333 | No |

SACO—Sequential analysis of chromatin occupation

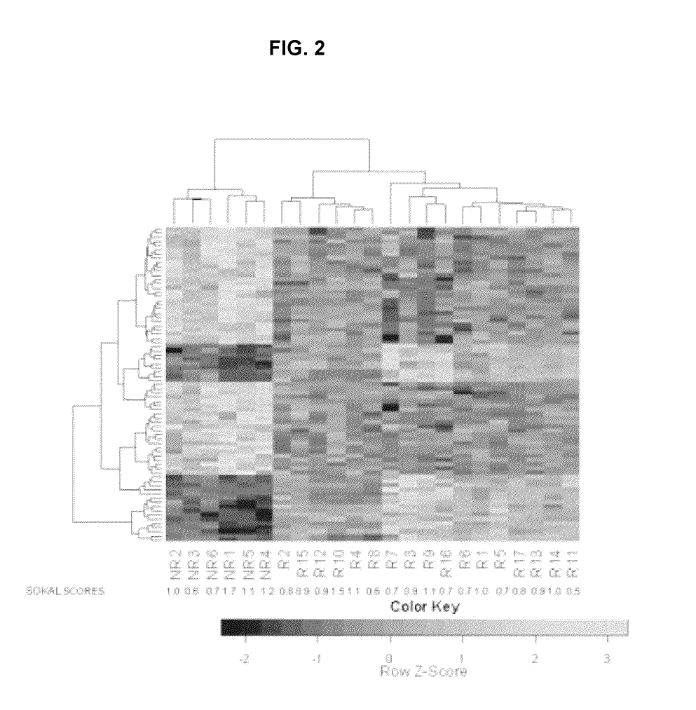

Validation of the response classifier in an independent test sample: For validation, CD34+ cells were prospectively collected from 23 newly diagnosed chronic phase patients prior to starting imatinib. Seventeen (74%) of these patients achieved CCyR within 12 months (Table 3), in keeping with the results of the IRIS study (O'Brien et al., N Engl J Med 348(11):994-1004, 2003). Microarray analysis was carried out using the same protocol as for the training set. As with the training set, unsupervised cluster analysis using the 75-probe set classifier was performed first. Responders were readily separated from non-responders (FIG. 2). Next, the prediction algorithm was applied to the validation set. Correct predictions were made in 15/17 responders and 5/6 non-responders, for an estimated accuracy rate of 86.9% (Table 3).

TABLE 3
Sokal risk score, observed and predicted
response in the validation set

| | Sokal risk | Observed | Predicted |
|---|---|---|---|
| Patient # | score | response | response |
| V1 | 1.1 | R | R |
| V2 | 0.7 | R | R |
| V3 | 1.1 | NR | NR |
| V4 | 1.0 | R | R |
| V5 | 0.6 | NR | R |
| V6 | 0.9 | R | R |
| V7 | 0.7 | R | R |
| V8 | 0.9 | R | R |
| V9 | 0.7 | R | R |
| V10 | 0.8 | R | R |
| V11 | 0.5 | R | R |
| V12 | 0.9 | R | NR |
| V13 | 1.0 | R | R |
| V14 | 0.9 | R | R |
| V15 | 1.2 | NR | NR |
| V16 | 0.7 | NR | NR |
| V17 | 0.8 | R | R |
| V18 | 1.1 | R | R |
| V19 | 1.7 | NR | NR |
| V20 | 1.0 | NR | NR |
| V21 | 1.5 | R | NR |
| V22 | 0.7 | R | R |
| V23 | 0.6 | R | R |

NR—non responder; R—responder

Comparison with Sokal Scores: Patients with a high Sokal score (>1.2) have a lower probability of achieving CCyR. The relation between the Sokal score of the patients in the validation set and their classification by gene array was examined. All 10 patients with a low Sokal score (≦0.8), 7/11 patients with an intermediate Sokal score (>0.8; ≦1.2) and 0/2 patients with a high Sokal score (>1.2) were classified as responders (Table 3). To compare the ability of the Sokal score and the classifier to predict cytogenetic response, it was assumed that patients with a high Sokal risk would be non-responders, whereas patients with a low or intermediate risk would be responders. For 16 of the 23 subjects, both Sokal score and classifier correctly predicted response. In 2 patients, classifier and Sokal score made identical but incorrect predictions: patient #V21 (Sokal score 1.5), was misclassified as a non-responder and patient #V5 (Sokal score 0.6) was misclassified as a responder. Risk prediction for the remaining 5 subjects was discordant between classifier and Sokal score. The classifier correctly identified four patents as non-responders (#V3, V15, V16, V20), whose Sokal scores (1.1, 1.2, 0.7 and 1.0, respectively) predicted response, while one responder (#V12, Sokal risk 0.9) was misclassified as a non-responder. Thus, the classifier correctly identified ⅚ non-responders, compared to ⅙ based on Sokal criteria.

Functional Structure of the Classifier: To gain insight into mechanisms underlying primary cytogenetic resistance and develop an understanding of structure and regulation of the classifier genes, bioinformatics tools were applied to identify potential regulatory networks, focusing on the minimal classifier. Gene ontology (GO) analysis revealed overrepresentation of several functional groups (Table 4).

TABLE 4
Functional Gene Ontology Enrichment in Classifier Genes

| Classi- | | | |
|---|---|---|---|
| fication | Description | Genes | P-value |
| MF | receptor binding | CECR1, FCN1, ADM, ANGPT1, | 0.00319 |
| | | S100A10, VWF, CLEC7A, | |
| | | MUC4, EGFL6 | |
| MF | collagen binding | VWF, ITGA2 | 0.0365 |
| BP | cell adhesion | MMRN1, ITGA2, VWF, ITGB8, | 0.001 |
| | | EVA1, MUC4 | |
| BP | transcriptional | ZNF44, MEIS1, NFIB, CEBPB, | 0.02 |
| | regulation | MAFB, ZNF140, ZNF253 | |

BP—biological process; MF—molecular function

Genes related to ligand/receptor binding are significantly overrepresented (FDR adjusted p<0.003), including S100A10, ADM, CLEC7A, CECR1, FCN1 and ANGPT 1. Five of these transcripts were down- and four (VWF, ANGPT 1, EGFL6 and MUC4) were upregulated in non-responders compared to responders. A second group with significant overrepresentation is transcripts involved in cell adhesion (p<0.001). All 6 transcripts in this group (MMRN1, ITGA2, VWF, ITGB8, EVA1 and MUC4) were upregulated in nonresponders. A third cluster of transcripts with significant overrepresentation (p<0.02) is related to transcriptional regulation. Seven of these transcripts were upregulated [ZNF44, MEIS1, NFIB (3 different transcripts), ZNF140 and ZNF253] and two downregulated (CEBPB and MAFB) in non-responders.

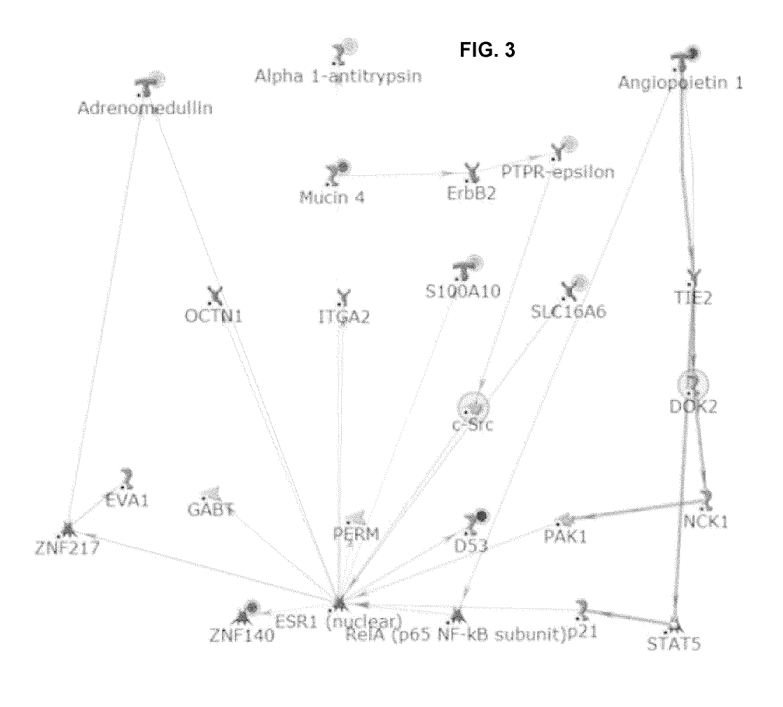

Pathway analysis. To identify regulatory networks, potential protein-protein interactions were examined among the members of the classifier, using the MetaCore Database™. Analysis of protein-protein interaction data identified a highly significant interaction subnetwork (p<4.85-36), which included two ANGPT1 signaling related pathways (both part of MetaCore Curated Map 532). The key classifier node that linked both of these pathways was ANGPT1, which had direct interactions with other key angiogenesis proteins in the subnetwork such as TIE2 (FIG. 3). Gene ontology analysis within the ANGPT1 subnetwork showed a highly significant overrepresentation (p<4.20-07) of proteins associated with transmembrane receptor protein tyrosine kinase signaling (GO:0007169). This annotation represents the series of molecular signals generated as a consequence of a transmembrane receptor tyrosine kinase binding their cognate ligands. The majority of the members with this GO annotation were also members of the ANGPT1-related pathways (FIG. 3). These data suggest that activation of tyrosine kinases through receptor binding and increased angiogenesis may contribute to primary cytogenetic resistance.

Involvement of β-catenin in the regulation of classifier genes: The rate of MCyR is highest in the chronic phase and lowest in blast crisis. Since activation of Wnt/β-catenin signaling in granulocyte/macrophage progenitor cells has been reported in cells from patients with blast crisis (Jamieson et al., N Engl J Med, 351(7):657-67, 2004) it was reasoned that genes associated with failure to achieve MCyR may be regulated by β-catenin, reflecting an advanced disease stage that is not yet visible morphologically. To test this hypothesis a library of β-catenin targets previously identified in by serial analysis of chromatin occupation (SACO) in a colon cancer cell line was used (Yochum et al., Proc Natl Acad Sci USA 104(9):3324-9, 2007). A significant enrichment of potential β-catenin targets was found in the classifier list compared to the remainder of the array (56% vs. 30.4% on array, p<0.001). Specifically, 62% of the up-regulated genes are β-catenin targets with TCF motifs either in the promoter or within the gene boundaries, suggesting that β-catenin activation in non-responders may be an important driver of the gene expression signature associated with primary cytogenetic resistance.

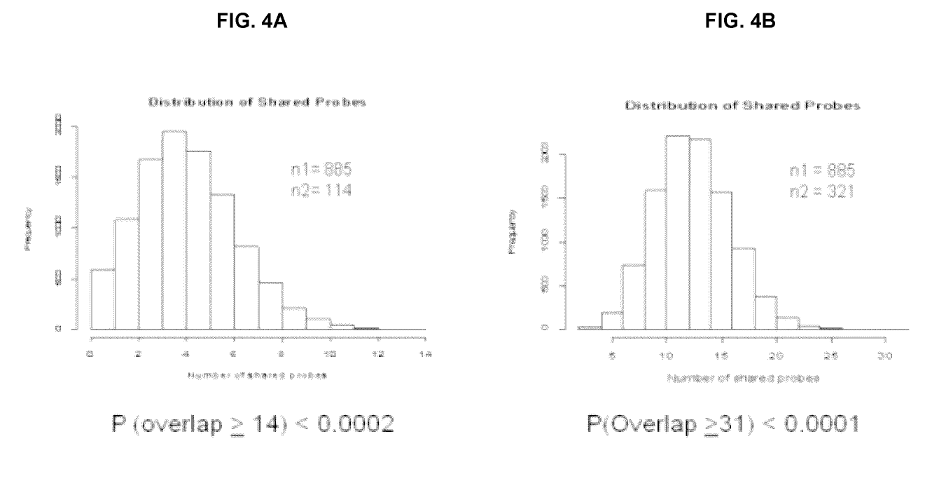

Comparison with published signatures of CD34+ CML cells: Two studies have reported expression signatures of CD34+ cells in relation to disease phase and duration of chronic phase in patients treated with non-imatinib therapy, respectively (19;20). To test whether primary cytogenetic resistance is a reflection of advanced disease, 885 response-related genes were analyzed for overlap with the published lists. For both the Zheng et al. (14 concordant transcripts, FIG. 4A) and Yong et al. (31 concordant transcripts, FIG. 4B) data, there was a highly significant overlap with our list of 885 transcripts. Five genes (CSTA, RNASE3, PRTN3, PLAUR, MPO, all downregulated in nonresponders) overlapped between the three data sets (Table 5).

TABLE 5
Overlap between gene signatures of non-response vs. response(current study),
short vs. long duration duration of chronic phase with non-imatinib therapy
(Young et al.) and blast crisis vs. chronic phase (Zheng et al.)

| Probeset | Gene Symbol | Current study | Yong et al. | Zheng et al. | Direction |
|---|---|---|---|---|---|
| 201693_s_at | EGR1 | + | + | − | UP |
| 202207_at | ARL4C | + | + | − | DOWN |
| 202708_s_at | HIST2H2BE | + | + | − | UP |
| 202912_at | ADM | + | + | − | DOWN |
| 203948_s_at | MPO | + | + | + | DOWN |
| 203973_s_at | CEBPD | + | + | − | DOWN |
| 204174_at | ALOX5AP | + | + | − | DOWN |
| 204971_at | CSTA | + | + | + | DOWN |
| 205382_s_at | CFD | + | + | − | DOWN |
| 205653_at | CTSG | + | + | − | DOWN |
| 205896_at | SLC22A4 | + | + | − | DOWN |
| 206851_at | RNASE3 | + | + | + | DOWN |
| 206871_at | ELA2 | + | + | − | DOWN |
| 207341_at | PRTN3 | + | + | + | DOWN |
| 209201_x_at | CXCR4 | + | + | − | DOWN |
| 210254_at | MS4A3 | + | + | − | DOWN |
| 210387_at | HIST1H2BG | + | + | − | UP |
| 210425_x_at | GOLGA8A/// | + | + | − | UP |
| | GOLGA8B | | | | |
| 210951_x_at | RAB27A | + | + | − | DOWN |
| 211919_s_at | CXCR4 | + | + | − | DOWN |
| 211924_s_at | PLAUR | + | + | + | DOWN |
| 214290_s_at | HIST2H2AA3/// | + | + | − | UP |
| | HIST2H2AA4 | | | | |
| 214469_at | HIST1H2AB/// | + | + | − | UP |
| | HIST1H2AE | | | | |
| 214472_at | HIST1H3D | + | + | − | UP |
| 214575_s_at | AZU1 | + | + | − | DOWN |
| 215071_s_at | HIST1H2AC | + | + | − | UP |
| 215779_s_at | HIST1H2BC/// | + | + | − | UP |
| | HIST1H2BE/// | | | | |
| | HIST1H2BF/// | | | | |
| | HIST1H2BG/// | | | | |
| | HIST1H2BI | | | | |
| 217028_at | CXCR4 | + | + | − | DOWN |
| 218280_x_at | HIST2H2AA3/// | + | + | − | UP |
| | HIST2H2AA4 | | | | |
| 221840_at | PTPRE | + | + | − | DOWN |
| 222067_x_at | HIST1H2BD | + | + | − | UP |
| 203372_s_at | SOCS2 | + | − | + | UP |
| 204232_at | FCER1G | + | − | + | DOWN |
| 204351_at | S100P | + | − | + | DOWN |
| 205863_at | S100A12 | + | − | + | DOWN |
| 211924_s_at | PLAUR | + | − | + | DOWN |
| 212501_at | CEBPB | + | − | + | DOWN |
| 213524_s_at | G0S2 | + | − | + | DOWN |
| 213537_at | HLA-DPA1 | + | − | + | UP |
| 219777-at | GIMAP6 | + | − | + | UP |

Gene Ontology Analysis: There is significant over-representation of transcripts in the minimal classifier that are related to receptor binding (FDR adjusted P<0.03). Transcripts in this classifier were also annotated for cell adhesion, protein binding, protease inhibitor binding etc. All six transcripts related to cell adhesion were up-regulated. There was also a subgroup of transcripts related to transcription. Five transcripts had apoptosis related GO annotation: three which induce or are associated with apoptosis, all of which are up-regulated, and two associated with anti-apoptosis both of which are down-regulated.

Pathway Analysis of minimal subset: The minimal subset list was examined to determine if there were subnetworks in pathways that were co-regulated. Four genes in the focal adhesion pathway were all up-regulated. Three of these transcripts are also involved in the ECM-receptor interaction pathway. The list also included genes involved in complement and coagulation cascades, induction of apoptosis through DR3 and DR4/5 Death Receptors, Regulation of ck1/cdk5 by type 1 glutamate receptors, p53 Signaling Pathway, Inhibition of Matrix Metalloproteinases, Hedgehog signaling, and IL 6 signaling pathway.

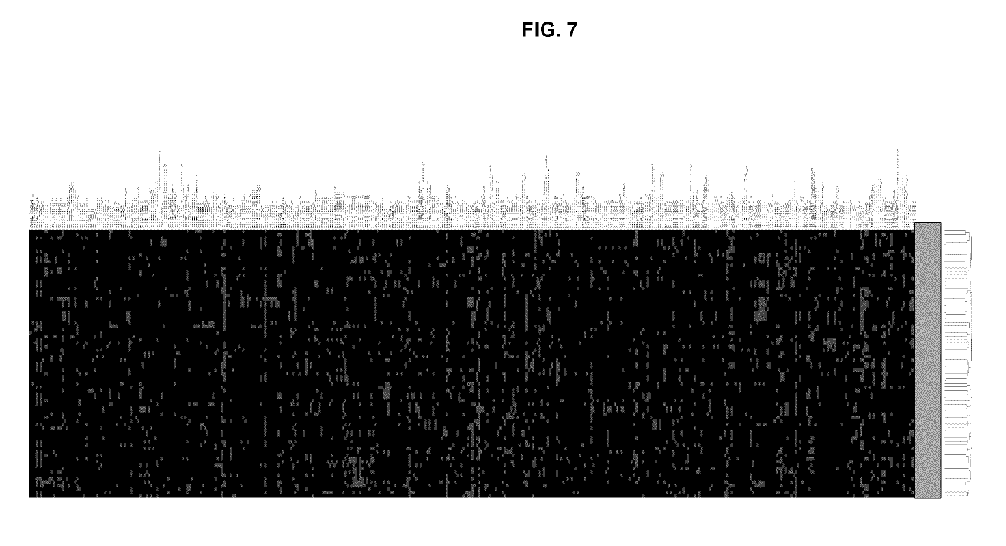

Promoter analysis: The 2 kb upstream sequences of the transcripts in the minimal classifier were retrieved and analyzed to determine which transcription factor binding sites were shared across the transcripts. A number of transcripts shared common binding sites (FIG. 7).

### Example 4

### Mechanism of Resistance to BCR-ABL Inhibitors

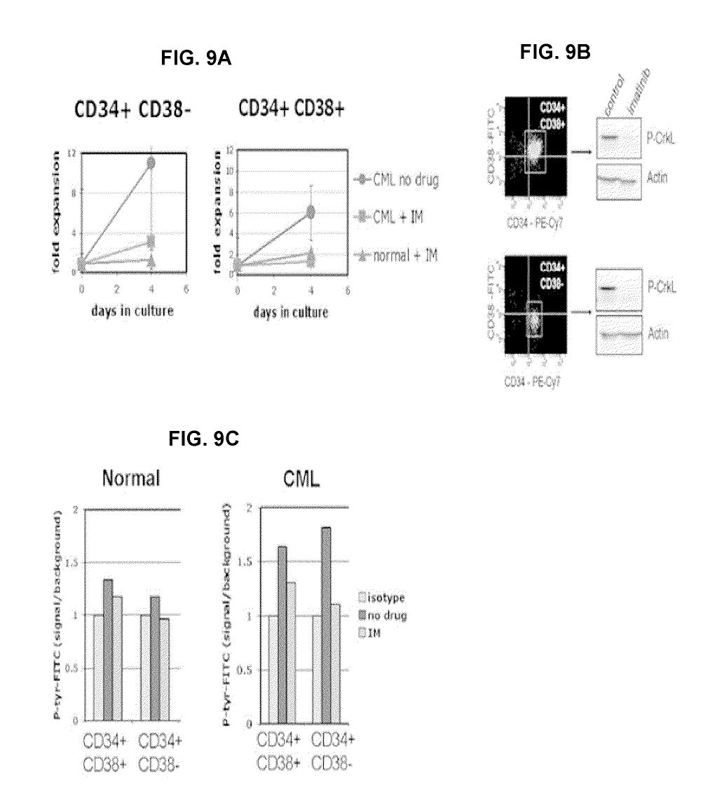

Few patients with kinase inhibitor resistance mutations were found in an analysis of complete cytogenetic responders for BCR-ABL kinase domain mutation. In addition, even in the few patient that such mutation were detected, most of these mutant clones were only detected transiently and did not lead to relapse. This suggests that kinase domain mutations are not a common mechanism of disease persistence. Since no technology is available to enrich for persistent leukemia cells, the analysis disclosed herein focuses on CML cells from newly diagnosed patients treated ex vivo with imatinib. A combination of lineage-depletion columns and high speed sorting was used to select highly pure populations of Lin−/CD34+/CD38+(enriched for progenitor cells) and Lin−/CD34+/CD38+ cells (enriched for stem cells). These cells were cultured in medium containing physiological concentrations of cytokines and 5 μM imatinib. In preliminary experiments (N=4) it was observed that growth was reduced to approximately the level of normal cells (FIG. 9A), but viability was maintained. To understand whether the cells survive because imatinib fails to suppress BCR-ABL activity we measured total phosphotyrosine levels by FACS and phosphorylation of CrkL, a specific substrate of BCR-ABL, by immunoblot. Imatinib reduced total phosphotyrosine and phospho-CrkL to levels similar to those of normal cells of identical immunophenotype (FIG. 9B+C). It was concludes that survival of primitive CML cells may not require BCR-ABL kinase activity, implicating BCR-ABL-independent extrinsic or intrinsic mechanisms in the maintenance of viability.

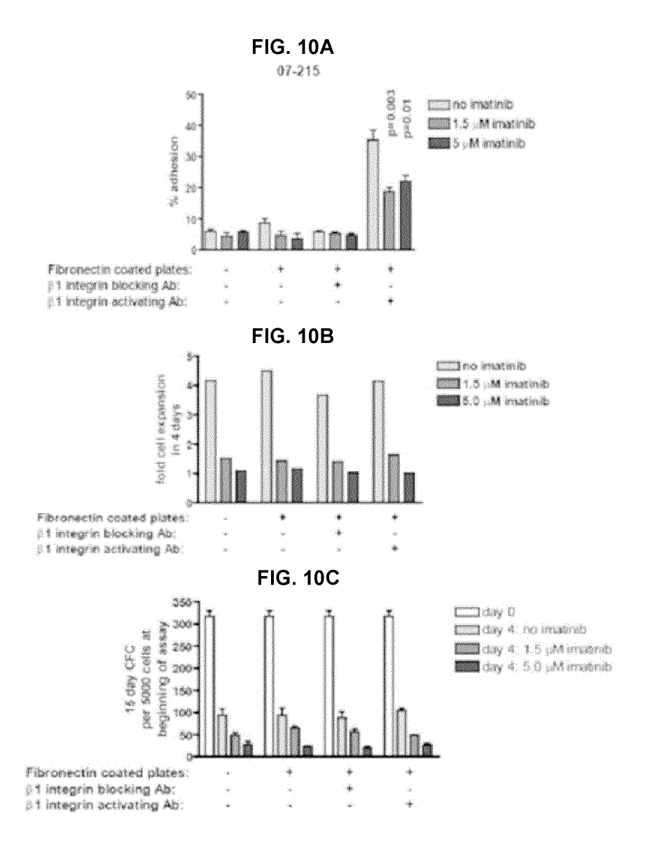

We have investigated whether adhesion to fibronectin may promote survival of CML progenitor cells in the presence of imatinib, as had been suggested from studies in cell lines. To reliably quantify adherence we modified the McClay (1981 PNAS 78:4975-9) centrifugal adhesion assay, using fluorescently labeled cells. CML CD34+ cells showed little spontaneous adhesion, which was further reduced by imatinib. Adhesion increased upon treatment with a betal integrin activating antibody (B44, Millipore) and was again reduced by imatinib. However, adhesion to integrin did not influence the recovery of viable cells and colony-forming cells (CFU-GM) (N=3, FIG. 10).

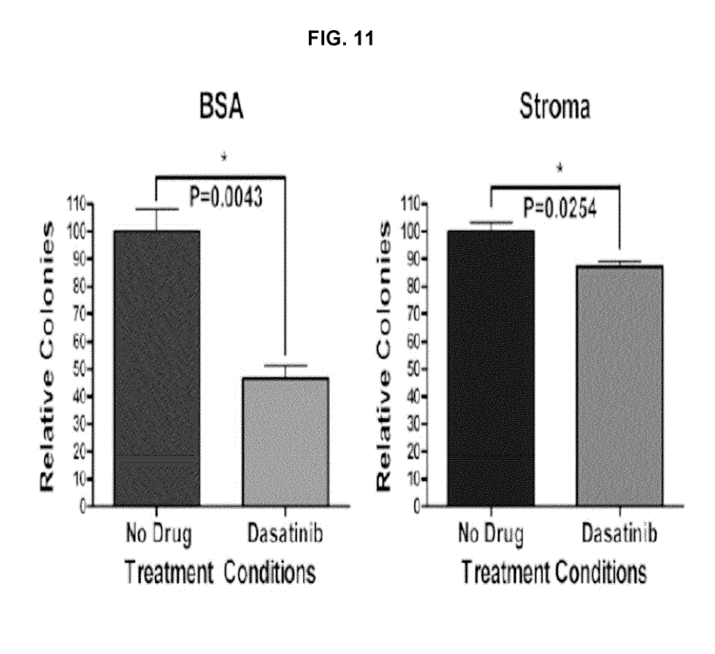

In an independent experiment, fibronectin-adherent and non-adherent fractions were analyzed separately for apoptosis in response to 50 nM dasatinib, but no differences were detected. However, when CD34+ cells from the same patient were cultured on a stromal cell layer, there was almost complete protection of CFU-GM activity, suggesting that co-culture with stromal cells but not adhesion to fibronectin protects CML cells from dasatinib (FIG. 11).

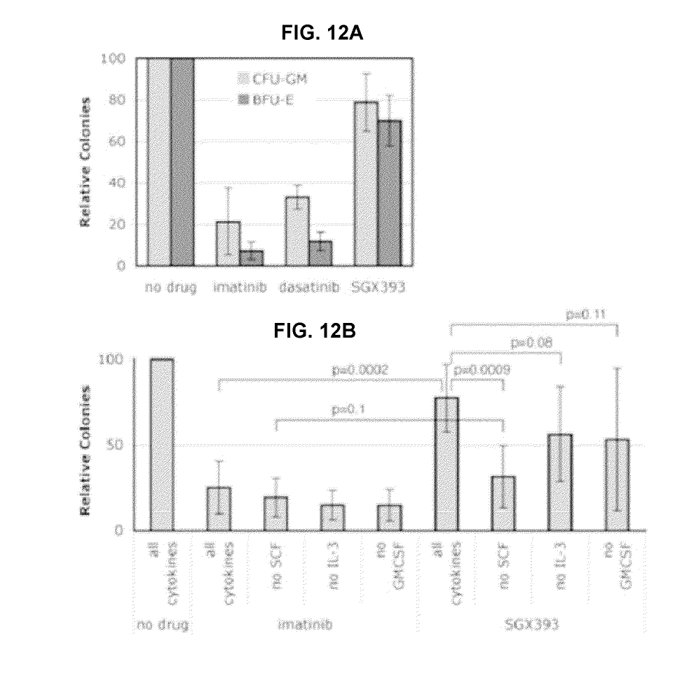

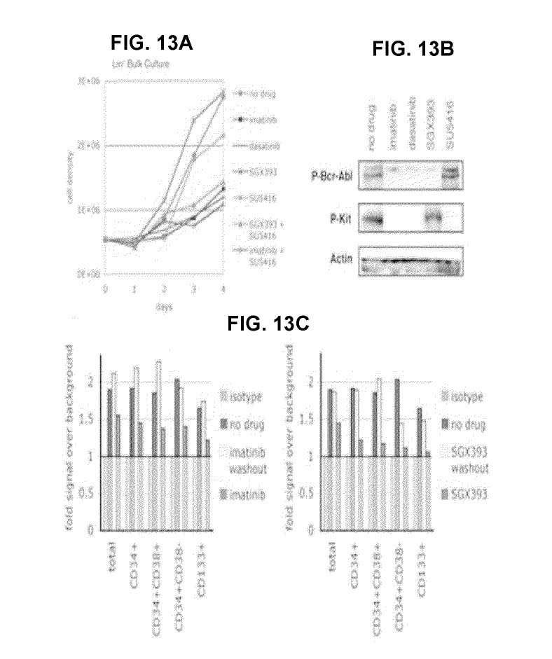

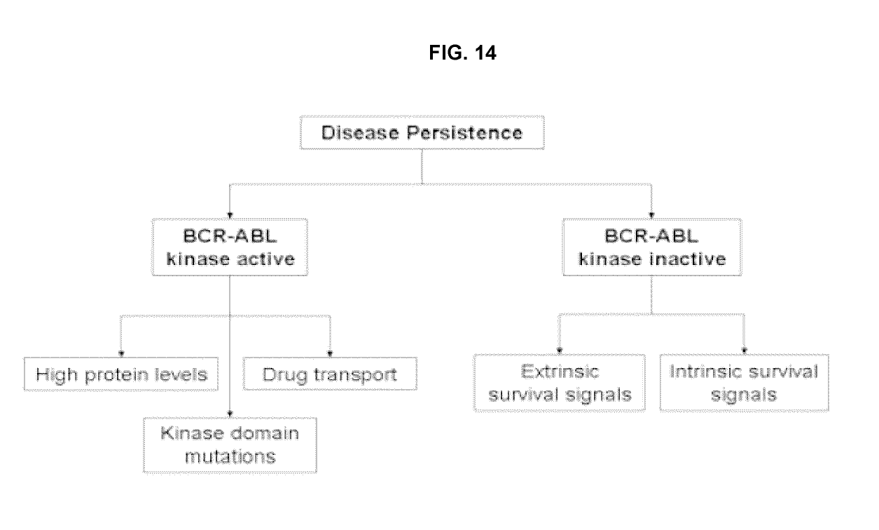

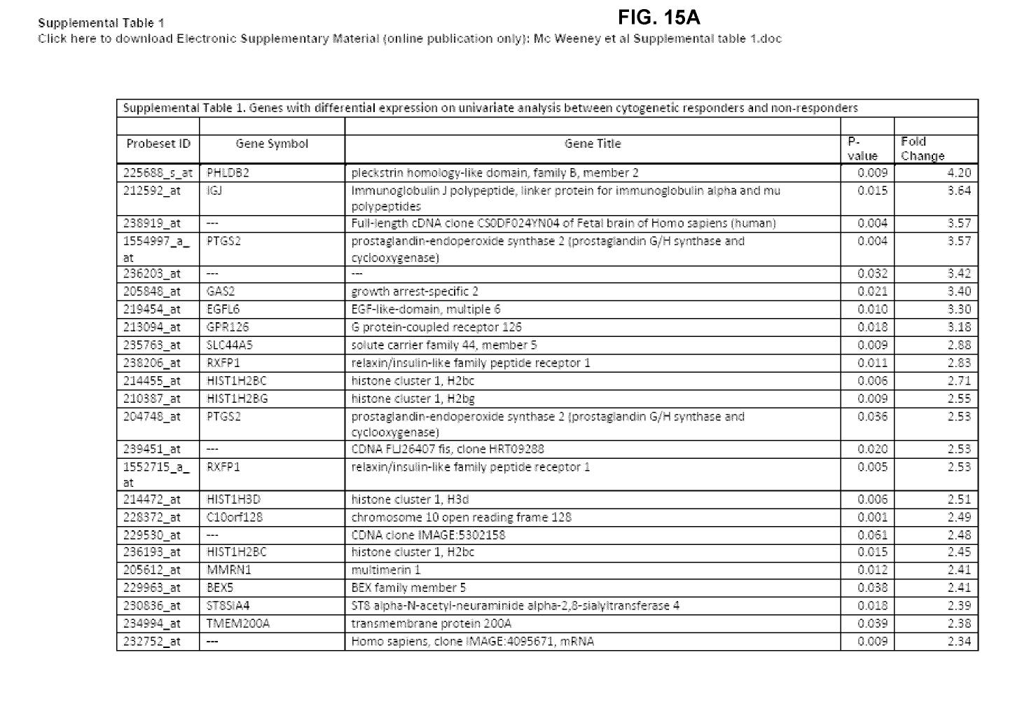

SCF increased the activity of SGX70393, but not imatinib (FIG. 12B). These results were confirmed in Lin−/CD34+ CML cells. SGX70393 had minimal effects alone, although it reduced pCrkL levels to similar degree in primitive and more differentiated CML cells (FIG. 13A+C). However, combination with SU5416 (an inhibitor of KIT but not BCR-ABL, FIG. 13B) reduced proliferation to the level seen with imatinib, suggesting that imatinib's ability to suppress the growth of human primitive CML cells is dependent on its ability to inhibit KIT. This raised the question whether mutations or polymorphisms of KIT might influence the sensitivity of cells to imatinib. Thus far, we have sequenced the coding region of KIT in 12 patients with acquired imatinib resistance and various proportions of Ph+ metaphases (but without ABL kinase domain mutations), and 9 imatinib-naïve patients in chronic phase. Potential mutations were detected in 3/9 imatinib-naive patients and included the extracellular, juxtamembrane and tyrosine kinase domain. Interestingly, all 12 patients with acquired resistance, but only 4/9 imatinib-naïve patients expressed exclusively the GNNK− isoform of KIT (P=0.006). The GNNK− isoform is a juxtamembrane domain splice variant with enhanced signaling compared to the GNNK+ isoform and the capacity to transform fibroblasts upon ligand binding. Thus, inhibition of KIT can be important for imatinib's activity and that mutations or splice variants of KIT may modulate the response of CML cells to imatinib. Thus, assays of KIT activity could be used to detect subjects resistant to treatment with a BCR-ABL inhibitor.

### Example 5

### Nucleotide Sequences of the Genes Listed in Table 2

PHLDB2: Pleckstrin homology-like domain, family B, member 2. Exemplary nucleic acid sequences of PHLDB2 can be found on GENBANK® at accession nos. NM_{—}001134439, NM_{—}001134438, and NM_{—}001134437, as available Dec. 6, 2007, incorporated herein by reference in their entirety.

GAS2: Growth arrest-specific 2. Exemplary nucleic acid sequences of GAS2 can be found on GENBANK® at accession nos. NM_{—}005256 and NM_{—}177553, as available Dec. 6, 2007, incorporated herein by reference in their entirety.

EGFL6: EGF-like-domain, multiple 6. An exemplary nucleic acid sequence of EGFL6 can be found on GENBANK® at accession no. NM_{—}015507, as available Dec. 6, 2007, incorporated herein by reference in its entirety.

RXFP1: Relaxin/insulin-like family peptide receptor 1. An exemplary nucleic acid sequence of RXFP1 can be found on GENBANK® at accession no. NM_{—}021634, as available Dec. 6, 2007, incorporated herein by reference in its entirety.

MMRN1: Multimerin 1. An exemplary nucleic acid sequence of MMRN1 can be found on GENBANK® at accession no. NM_{—}007351, as available Dec. 6, 2007, incorporated herein by reference in its entirety.

NGFRAP1L1: Brain expressed, X-linked 5. An exemplary nucleic acid sequence of NGFRAP1L1 can be found on GENBANK® at accession no. NM_{—}001012978, as available Dec. 6, 2007, incorporated herein by reference in its entirety.

SPOCK3: Sparc/osteonectin, cwcv and kazal-like domains proteoglycan (testican) 3. Exemplary nucleic acid sequences of SPOCK3 can be found on GENBANK® at accession nos. NM_{—}001040159 and NM_{—}016950, as available Dec. 6, 2007, incorporated herein by reference in their entirety.

KIF21A: Kinesin family member 21A kinesin. An exemplary nucleic acid sequence of KIF21A can be found on GENBANK® at accession no. NM_{—}017641, as available Dec. 6, 2007, incorporated herein by reference in its entirety.

FLJ12033: An exemplary nucleic acid sequence of FLJ12033 can be found on GENBANK® at accession no. AK022095, as available Dec. 6, 2007, incorporated herein by reference in its entirety.

ANGPT1: Angiopoietin 1. An exemplary nucleic acid sequence of ANGPT1 can be found on GENBANK® at accession no. NM_{—}001146, as available Dec. 6, 2007, incorporated herein by reference in its entirety.

TMEM163: Transmembrane protein 163. An exemplary nucleic acid sequence of TMEM163 can be found on GENBANK® at accession no. NM_{—}030923, as available Dec. 6, 2007, incorporated herein by reference in its entirety.

EMCN: Endomucin. An exemplary nucleic acid sequence of EMCN can be found on GENBANK® at accession no. NM_{—}016242, as available Dec. 6, 2007, incorporated herein by reference in its entirety.

ITGA2: Integrin, alpha 2. An exemplary nucleic acid sequence of ITGA2 can be found on GENBANK® at accession no. NM_{—}002203, as available Dec. 6, 2007, incorporated herein by reference in its entirety.

CLIP4: CAP-GLY domain containing linker protein family, member 4. An exemplary nucleic acid sequence of CLIP4 can be found on GENBANK® at accession no. NM_{—}024692, as available Dec. 6, 2007, incorporated herein by reference in its entirety.

SH3GL3: SH3-domain GRB2-like 3. An exemplary nucleic acid sequence of SH3GL3 can be found on GENBANK® at accession no. NM_{—}003027, as available Dec. 6, 2007, incorporated herein by reference in its entirety.

SLC8A3: Solute carrier family 8 (sodium/calcium exchanger), member 3. Exemplary nucleic acid sequences of SLC8A3 can be found on GENBANK® at accession nos. NM_{—}001130417, NM_{—}183002, NM_{—}033262, NM_{—}182936, NM_{—}182932, and NM_{—}058240, as available Dec. 6, 2007, incorporated herein by reference in their entirety.

PRKG1: Protein kinase, cGMP-dependent, type I. Exemplary nucleic acid sequences of PRKG1 can be found on GENBANK® at accession nos. NM_{—}001098512 and NM_{—}006258, as available Dec. 6, 2007, incorporated herein by reference in their entirety.

GPRASP2: G protein-coupled receptor associated sorting protein 2. Exemplary nucleic acid sequences of GPRASP2 can be found on GENBANK® at accession nos. NM_{—}001004051 and NM_{—}138437, as available Dec. 6, 2007, incorporated herein by reference in their entirety.

VWF: Von Willebrand factor. An exemplary nucleic acid sequence of VWF can be found on GENBANK® at accession no. NM_{—}000552, as available Dec. 6, 2007, incorporated herein by reference in its entirety.

BC041986: An exemplary nucleic acid sequence of BC041986 can be found on GENBANK® at accession no. BC041986, as available Dec. 6, 2007, incorporated herein by reference in its entirety.

HEMGN: Hemogen. An exemplary nucleic acid sequence of HEMGN can be found on GENBANK® at accession no. NM_{—}018437 and NM_{—}197978, as available Dec. 6, 2007, incorporated herein by reference in their entirety.

ZNF44: Zinc finger protein 44. An exemplary nucleic acid sequence of ZNF44 can be found on GENBANK® at accession no. NM_{—}016264, as available Dec. 6, 2007, incorporated herein by reference in its entirety.

MEIS1: Meis homeobox 1. An exemplary nucleic acid sequence of MEIS1 can be found on GENBANK® at accession no. NM_{—}002398, as available Dec. 6, 2007, incorporated herein by reference in its entirety.

CMAH: Cytidine monophosphate-N-acetylneuraminic acid hydroxylase. An exemplary nucleic acid sequence of CMAH can be found on GENBANK® at accession no. NR_{—}002174, as available Dec. 6, 2007, incorporated herein by reference in its entirety.

KIAA1598. Exemplary nucleic acid sequences of KIAA1598 can be found on GENBANK® at accession nos. NM_{—}018330 and NM_{—}001127211, as available Dec. 6, 2007, incorporated herein by reference in their entirety.

RP11-145H9.1: Myosin light chain kinase family, member 4. An exemplary nucleic acid sequence of RP11-145H9.1 can be found on GENBANK®at accession no. NM_{—}001012418, as available Dec. 6, 2007, incorporated herein by reference in its entirety.

RBPMS: RNA binding protein with multiple splicing. Exemplary nucleic acid sequences of RBPMS can be found on GENBANK® at accession nos. NM_{—}001008712, NM_{—}001008710, NM_{—}001008711, and NM_{—}006867, as available Dec. 6, 2007, incorporated herein by reference in their entirety.

MGC13057: Hypothetical protein MGC13057. Exemplary nucleic acid sequences of MGC13057 can be found on GENBANK® at accession nos. NM_{—}001042520, NM_{—}001042521, NM_{—}001042519, and NM_{—}032321, as available Dec. 6, 2007, incorporated herein by reference in their entirety.

NFIB: Nuclear factor I/B. An exemplary nucleic acid sequence of NFIB can be found on GENBANK® at accession no. NM_{—}005596, as available Dec. 6, 2007, incorporated herein by reference in its entirety.

ARMCX2: Armadillo repeat containing, X-linked 2. Exemplary nucleic acid sequences of ARMCX2 can be found on GENBANK® at accession nos. NM_{—}014782 and NM_{—}177949, as available Dec. 6, 2007, incorporated herein by reference in their entirety.

ITGB8: Integrin, beta 8. An exemplary nucleic acid sequence of ITGB8 can be found on GENBANK® at accession no. NM_{—}002214, as available Dec. 6, 2007, incorporated herein by reference in its entirety.

CALN1: Calneuron 1. An exemplary nucleic acid sequence of CALN1 can be found on GENBANK® at accession no. NM_{—}031468 NM_{—}001017440, as available Dec. 6, 2007, incorporated herein by reference in their entirety.

MPDZ: Multiple PDZ domain protein. Exemplary nucleic acid sequences of MPDZ can be found on GENBANK® at accession nos. NM_{—}032622 and NM_{—}001126328, as available Dec. 6, 2007, incorporated herein by reference in their entirety.

EVA1: Myelin protein zero-like 2. Exemplary nucleic acid sequences of EVA1 can be found on GENBANK® at accession nos. NM_{—}144765 and NM_{—}005797, as available Dec. 6, 2007, incorporated herein by reference in their entirety.

LOH11CR2A: Von Willebrand factor A domain containing 5A. Exemplary nucleic acid sequences of LOH11CR2A can be found on GENBANK®at accession nos. NM_{—}001130142, NM_{—}014622, and NM_{—}198315, as available Dec. 6, 2007, incorporated herein by reference in their entirety.

MOSC2: MOCO sulphurase C-terminal domain containing 2. An exemplary nucleic acid sequence of MOSC2 can be found on GENBANK® at accession no. NM_{—}017898, as available Dec. 6, 2007, incorporated herein by reference in its entirety.

ZNF140: Zinc finger protein 140. An exemplary nucleic acid sequence of ZNF140 can be found on GENBANK® at accession no. NM_{—}003440, as available Dec. 6, 2007, incorporated herein by reference in its entirety.

ABAT: 4-aminobutyrate aminotransferase. Exemplary nucleic acid sequences of ABAT can be found on GENBANK® at accession nos. NM_{—}001127448, NM_{—}000663, and NM_{—}020686, as available Dec. 6, 2007, incorporated herein by reference in their entirety.

C5orf25: Chromosome 5 open reading frame 25. An exemplary nucleic acid sequence of C5orf25 can be found on GENBANK® at accession no. NM_{—}198567, as available Dec. 6, 2007, incorporated herein by reference in its entirety.

KLHL13: Kelch-like 13. An exemplary nucleic acid sequence of KLHL13 can be found on GENBANK® at accession no. NM_{—}033495, as available Dec. 6, 2007, incorporated herein by reference in its entirety.

MUC4: Mucin 4, cell surface associated. Exemplary nucleic acid sequences of MUC4 can be found on GENBANK® at accession nos. NM_{—}018406, NM_{—}138297, and NM_{—}004532, as available Dec. 6, 2007, incorporated herein by reference in their entirety.

TPD52L1: Tumor protein D52-like 1. Exemplary nucleic acid sequences of TPD52L1 can be found on GENBANK® at accession nos. NM_{—}001003395 NM_{—}001003397 NM_{—}003287, and NM_{—}001003396, as available Dec. 6, 2007, incorporated herein by reference in their entirety.

TIMP3: TIMP metallopeptidase inhibitor 3. An exemplary nucleic acid sequence of TIMP3 can be found on GENBANK® at accession no. NM_{—}000362, as available Dec. 6, 2007, incorporated herein by reference in its entirety.

BC043173: An exemplary nucleic acid sequence of BC043173 can be found on GENBANK® at accession no. BC043173, as available Dec. 6, 2007, incorporated herein by reference in its entirety.

ZNF253: Zinc finger protein 253. An exemplary nucleic acid sequence of ZNF253 can be found on GENBANK® at accession no. NM_{—}021047, as available Dec. 6, 2007, incorporated herein by reference in its entirety.

CEBPB: CCAAT/enhancer binding protein (C/EBP), beta. An exemplary nucleic acid sequence of CEBPB can be found on GENBANK® at accession no. NM_{—}005194, as available Dec. 6, 2007, incorporated herein by reference in its entirety.

CECR1: Cat eye syndrome chromosome region, candidate 1. Exemplary nucleic acid sequences of CECR1 can be found on GENBANK® at accession nos. NM_{—}177405 and NM_{—}017424, as available Dec. 6, 2007, incorporated herein by reference in their entirety.

ARL4C: ADP-ribosylation factor-like 4C. An exemplary nucleic acid sequence of ARL4C can be found on GENBANK® at accession no. NM_{—}005737, as available Dec. 6, 2007, incorporated herein by reference in its entirety.

FLJ20273: RNA binding motif protein 47. Exemplary nucleic acid sequences of FLJ20273 can be found on GENBANK® at accession nos. NM_{—}001098634 and NM_{—}019027, as available Dec. 6, 2007, incorporated herein by reference in their entirety.

ADM: Adrenomedullin. An exemplary nucleic acid sequence of BC043173 can be found on GENBANK® at accession no. NM_{—}001124, as available Dec. 6, 2007, incorporated herein by reference in its entirety.

AI694722: An exemplary nucleic acid sequence of A1694722 can be found on GENBANK® at accession no. A1694722, as available Dec. 6, 2007, incorporated herein by reference in its entirety.

SLC22A4: Solute carrier family 22 (organic cation/ergothioneine transporter), member 4. An exemplary nucleic acid sequence of SLC22A4 can be found on GENBANK® at accession no. NM_{—}003059, as available Dec. 6, 2007, incorporated herein by reference in its entirety.

AF318321: An exemplary nucleic acid sequence of AF318321 can be found on GENBANK® at accession no. AF318321, as available Dec. 6, 2007, incorporated herein by reference in its entirety.

UPP1: Uridine phosphorylase 1. Exemplary nucleic acid sequences of UPP1 can be found on GENBANK® at accession nos. NM_{—}181597 and NM_{—}003364, as available Dec. 6, 2007, incorporated herein by reference in their entirety.

S100A10: S100 calcium binding protein A10. An exemplary nucleic acid sequence of S100A10 can be found on GENBANK® at accession no. NM_{—}002966, as available Dec. 6, 2007, incorporated herein by reference in its entirety.

P2RY5: Purinergic receptor P2Y, G-protein coupled, 5. An exemplary nucleic acid sequence of P2RY5 can be found on GENBANK® at accession no. NM_{—}005767, as available Dec. 6, 2007, incorporated herein by reference in its entirety.

IFI30: Interferon, gamma-inducible protein 30. An exemplary nucleic acid sequence of IFI30 can be found on GENBANK® at accession no. NM_{—}006332, as available Dec. 6, 2007, incorporated herein by reference in its entirety.

PTPRE: Protein tyrosine phosphatase, receptor type, E. Exemplary nucleic acid sequences of PTPRE can be found on GENBANK® at accession nos. NM_{—}006504 and NM_{—}130435, as available Dec. 6, 2007, incorporated herein by reference in their entirety.

CLEC7A: C-type lectin domain family 7, member A. Exemplary nucleic acid sequences of CLEC7A can be found on GENBANK® at accession nos. NM_{—}197954, NM_{—}197950, NM_{—}197949, NM_{—}197948, NM_{—}022570, and NM_{—}197947, as available Dec. 6, 2007, incorporated herein by reference in their entirety.

SERPINA1: Serpin peptidase inhibitor, Glade A (alpha-1 antiproteinase, antitrypsin). Exemplary nucleic acid sequences of SERPINA1 can be found on GENBANK® at accession nos. NM_{—}001127702, NG_{—}008290, NM_{—}001127707, NM_{—}001127706, NM_{—}001127705, NM_{—}001127704, NM_{—}001127703, NM_{—}001127701, NM_{—}001127700, NM_{—}001002236, NM_{—}001002235, and NM_{—}000295, as available Dec. 6, 2007, incorporated herein by reference in their entirety.

CTSG: Cathepsin G. An exemplary nucleic acid sequence of CTSG can be found on GENBANK® at accession no. NM_{—}001911, as available Dec. 6, 2007, incorporated herein by reference in its entirety.

SLC16A6: Solute carrier family 16, member 6 (monocarboxylic acid transporter 7). An exemplary nucleic acid sequence of SLC16A6 can be found on GENBANK® at accession no. NM_{—}004694, as available Dec. 6, 2007, incorporated herein by reference in its entirety.

MAFB: V-maf musculoaponeurotic fibrosarcoma oncogene homolog B. An exemplary nucleic acid sequence of MAFB can be found on GENBANK® at accession no. NM_{—}005461, as available Dec. 6, 2007, incorporated herein by reference in its entirety.

MPO: Myeloperoxidase. An exemplary nucleic acid sequence of MPO can be found on GENBANK® at accession no. NM_{—}000250, as available Dec. 6, 2007, incorporated herein by reference in its entirety.

FLJ22662: Hypothetical protein FLJ22662. An exemplary nucleic acid sequence of FLJ22662 can be found on GENBANK® at accession no. NM_{—}024829, as available Dec. 6, 2007, incorporated herein by reference in its entirety.

CSTA: Cystatin A. An exemplary nucleic acid sequence of CSTA can be found on GENBANK® at accession no. NM_{—}005213, as available Dec. 6, 2007, incorporated herein by reference in its entirety.

MS4A3: Membrane-spanning 4-domains, subfamily A, member 3. An exemplary nucleic acid sequence of MS4A3 can be found on GENBANK® at accession no. NM_{—}001031666, as available Dec. 6, 2007, incorporated herein by reference in its entirety.

FCN1: Ficolin. An exemplary nucleic acid sequence of FCN1 can be found on GENBANK® at accession no. NM_{—}002003, as available Dec. 6, 2007, incorporated herein by reference in its entirety.

It will be apparent that the precise details of the methods or compositions described may be varied or modified without departing from the spirit of the described invention. We claim all such modifications and variations that fall within the scope and spirit of the claims below.

We claim:

1. A method for determining if a subject diagnosed with chronic myelogenous leukemia (CML) will respond to treatment with BCR-ABL inhibitor, comprising:
assaying expression of at least five genes of PHLDB2, GAS2, EGFL6, RXFP1, MMRN1, NGFRAP1L1, SPOCK3, KIF21A, FLJ12033, ANGPT1, TMEM163, EMCN, ITGA2, CLIP4, SH3GL3, SLC8A3, PRKG1, GPRASP2, VWF, BC041986, HEMGN, ZNF44, MEIS1, CMAH, KIAA1598, RP11-145H9.1, RBPMS, MGC1305, NFIB, ARMCX2, ITGB8, CALN1, MPDZ, EVA1, LOH11CR2A, MOSC2, ZNF140, ABAT, C5orf25, KLHL13, MUC4, TPD52L1, TIMP3, BC043173, ZNF253, CEBPB, CECR1, ARL4C, FLJ20273, ADM, AI694722, SLC22A4, AF318321, UPP1, S100A10, P2RY5, IFI30, PTPRE, CLEC7A, SERPINA1, CTSG, SLC16A6, MAFB, MPO, FLJ22662, CSTA, MS4A3, and FCN1 from CD34+ cells isolated from the subject; and
comparing the expression of the at least five genes in the sample to a control,
wherein altered expression of the at least five genes as compared to the control predicts whether the subject will respond to treatment with the BCR-ABL inhibitor.

2. The method of claim 1, wherein the at least five genes is selected from the group consisting of PHLDB2, GAS2, EGFL6, RXFP1, MMRN1, NGFRAP1L1, SPOCK3, KIF21A, FLJ12033, ANGPT1, TMEM163, EMCN, ITGA2, CLIP4, SH3GL3, SLC8A3, PRKG1, GPRASP2, VWF, BC041986, HEMGN, ZNF44, MEIS1, CMAH, KIAA1598, RP11-145H9.1, RBPMS, MGC1305, NFIB, ARMCX2, ITGB8, CALN1, MPDZ, EVA1, LOH11CR2A, MOSC2, ZNF140, ABAT, C5orf25, KLHL13, MUC4, TPD52L1, TIMP3, BC043173, ZNF253, CEBPB, CECR1, ARL4C, FLJ20273, ADM, AI694722, SLC22A4, AF318321, UPP1, S100A10, P2RY5, IFI30, PTPRE, CLEC7A, SERPINA1, CTSG, SLC16A6, MAFB, MPO, FLJ22662, CSTA, MS4A3, and FCN1.

3. The method of claim 1, wherein the method comprises assaying expression of all of PHLDB2, GAS2, EGFL6, RXFP1, MMRN1, NGFRAP1L1, SPOCK3, KIF21A, FLJ12033, ANGPT1, TMEM163, EMCN, ITGA2, CLIP4, SH3GL3, SLC8A3, PRKG1, GPRASP2, VWF, BC041986, HEMGN, ZNF44, MEIS1, CMAH, KIAA1598, RP11-145H9.1, RBPMS, MGC1305, NFIB, ARMCX2, ITGB8, CALN1, MPDZ, EVA1, LOH11CR2A, MOSC2, ZNF140, ABAT, C5orf25, KLHL13, MUC4, TPD52L1, TIMP3, BC043173, ZNF253, CEBPB, CECR1, ARL4C, FLJ20273, ADM, AI694722, SLC22A4, AF318321, UPP1, S100A10, P2RY5, IFI30, PTPRE, CLEC7A, SERPINA1, CTSG, SLC16A6, MAFB, MPO, FLJ22662, CSTA, MS4A3, and FCN1.

4. The method of claim 1, wherein the prediction has an accuracy of at least 70%.

5. The method of claim 1, wherein the BCR-ABL inhibitor is imatinib, AMN107 (nilotinib), dasatinib, NS-187, ON012380, Bosutinib (SKI-606), INNO-406 (NS-187), and MK-0457 (VX-680), SGX70393 or BMS-354825.

6. The method of claim 1, wherein the control is a set of standard values indicating that a subject will respond to treatment with the BCR-ABL inhibitor.

7. The method of claim 6, wherein altered expression in the at least five genes relative to the control indicates that the subject will not respond to the BCR-ABL inhibitor.

8. The method of claim 6, wherein altered expression of the at least five genes relative to the control indicates that the first subject has a poor prognosis.

9. The method of claim 1, wherein the control is a set of standard values indicating that a subject will not respond to treatment with the BCR-ABL inhibitor.

10. The method of claim 9, wherein altered expression in the at least five genes relative to the control indicates that the subject will respond to the BCR-ABL inhibitor.

11. The method of claim 9, wherein altered expression of the at least five genes relative to the control indicates that the subject has a good prognosis.

12. The method of claim 1, wherein evaluating expression of the at least five genes comprises the use of a prediction analysis of microarrays (PAM).

13. The method of claim 1, wherein the control is the expression of the at least five genes from CD34+ cells isolated from a second subject with chronic myelogenous leukemia (CML), wherein the second subject responds to the BCR-ABL inhibitor.

14. The method of claim 13, wherein the second subject has a complete cytogenetic response.

15. The method of claim 13, wherein altered expression in the at least five genes relative to the control indicates that the subject will not respond to the BCR-ABL inhibitor.

16. The method of claim 13, wherein altered expression of the at least five genes relative to the control indicates that the subject has a poor prognosis.

17. The method of claim 1, wherein the control is the expression of the at least five genes from CD34+ cells isolated from a second subject with chronic myelogenous leukemia (CML), wherein the second subject does not respond to the BCR-ABL inhibitor.

18. The method of claim 17, wherein altered expression in the at least five genes relative to the control indicates that the subject will respond to the BCR-ABL inhibitor.

19. The method of claim 17, wherein altered expression of the at least five genes relative to the control indicates that the first subject has a good prognosis.

20. The method of claim 1, wherein assaying expression of the at least five genes comprises detecting mRNA.

21. The method of claim 20, wherein detecting mRNA comprises using a reverse-transcription-polymerase chain reaction (RT-PCR).

22. The method of claim 21, wherein the RT-PCR comprises quantitative RT-PCR.

23. The method of claim 1, wherein assaying expression of the at least five genes comprises using a microarray.

24. The method of claim 1, wherein assaying the expression of the at least five genes comprises detecting protein.

25. The method of claim 1, wherein the subject is a human.

26. The method of claim 1, wherein detecting whether there is altered expression of the at least five genes comprises evaluating a gene expression profile from the subject.

27. An array consisting of probes that specifically hybridize to PHLDB2, GAS2, EGFL6, RXFP1, MMRN1, NGFRAP1L1, SPOCK3, KIF21A, FLJ12033, ANGPT1, TMEM163, EMCN, ITGA2, CLIP4, SH3GL3, SLC8A3, PRKG1, GPRASP2, VWF, BC041986, HEMGN, ZNF44, MEIS1, CMAH, KIAA1598, RP11-145H9.1, RBPMS, MGC1305, NFIB, ARMCX2, ITGB8, CALN1, MPDZ, EVA1, LOH11CR2A, MOSC2, ZNF140, ABAT, C5orf25, KLHL13, MUC4, TPD52L1, TIMP3, BC043173, ZNF253, CEBPB, CECR1, ARL4C, FLJ20273, ADM, AI694722, SLC22A4, AF318321, UPP1, S100A10, P2RY5, IFI30, PTPRE, CLEC7A, SERPINA1, CTSG, SLC16A6, MAFB, MPO, FLJ22662, CSTA, MS4A3, and FCN1 nucleic acids.
